(19) United States
(12) Patent Application Publication
(10) Pub. No.: US 2026/0083701 A1
(43) Pub. Date: Mar. 26, 2026

(54) COMPOSITIONS, FORMULATIONS, AND METHODS FOR TREATMENT OF INFLAMMATORY CONDITIONS

(71) Applicant: Oddity Labs, LLC, New York, NY (US)

(72) Inventors: Enzo Corey BENFANTI, New York, NY (US); Avinash BOPPANA, New York, NY (US); Kongyu ZHANG, New York, NY (US); Evan ZHAO, New York, NY (US); Connor Wilson COLEY, New York, NY (US)

(21) Appl. No.: 19/341,739

(22) Filed: Sep. 26, 2025

Related U.S. Application Data
(63) Continuation of application No. PCT/US2024/022210, filed on Mar. 29, 2024.
(60) Provisional application No. 63/493,279, filed on Mar. 30, 2023.

(51) Int. Cl. A61K 31/40; A61K 9/00; A61K 9/06; A61K 31/4025; A61K 31/403; A61K 31/437; A61K 31/5025; A61K 31/5377; A61P 17/00

(52) U.S. Cl. CPC A61K 31/40; A61K 9/0014; A61K 9/06; A61K 31/4025; A61K 31/403; A61K 31/437; A61K 31/5025; A61K 31/5377; A61P 17/00

(57) ABSTRACT

Compounds and compositions for relief, management, or treatment of inflammation conditions, disorders, or diseases are provided herein. Methods for treatment of inflammatory conditions or disorders are also provided herein. The methods may reduce dryness, itching, redness, roughness, or flakiness of skin.

### CROSS REFERENCE

This is a continuation application which claims priority to International Application No. PCT/US2024/022210 filed Mar. 29, 2024, which claims the benefit of U.S. Provisional Application No. 63/493,279 filed Mar. 30, 2023, each of which is incorporated by reference herein in its entirety.

### SUMMARY

Interleukins-4 (IL-4) and Interleukins-13 (IL-13) may play roles in type-2-driven inflammatory skin conditions or disorders with complex pathophysiology, epidermal barrier dysfunction, IgE sensitization, intertwining immune dysregulation, environmental factors, and genetic predisposition. Atopic dermatitis (AD) is an example of an inflammatory skin condition or disorder. Despite efforts to develop therapeutic approaches for treatment of inflammatory skin conditions or disorders, there remains an unmet need for approaches that overcome inherent limitations of conventional methods. The present disclosure addresses these needs, for example by using small molecule inhibitors, and offers related advantages.

The present disclosure provides a method for treating, preventing, or providing relief for a disease, disorder, or condition in a subject in need thereof, comprising administering the subject a composition comprising a compound having a structure of Formula (I) or salt thereof,

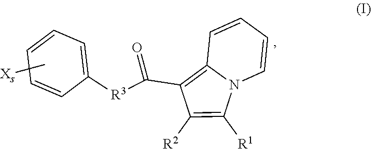

wherein
R¹ is selected from optionally substituted C₁₋₆ alkyl, C₂₋₆ alkenyl, and C₂₋₆ alkynyl, —OR^{1a}, —SR^{1a}, —C(O)R^{1a}, —C(O)OR^{1a}, —OC(O)R^{1a}, —OC(O)N(R^{1a})₂, —NR^{1a}S(O)₂R^{1a}, —C(O)N(R^{1a})₂, —S(O)₂(R^{1a}), —S(O)₂N(R^{1a})₂, optionally substituted C₃-C₁₂ carbocycle, and optionally substituted 5- to 14-membered heterocycle;
   each R^{1a} is independently selected at each occurrence from C₁₋₆ alkyl, C₅-C₈ carbocycle, and C₅-C₈ heteroaryl, each of which is optionally substituted with one or more substituents selected from halogen, —NO₂, —NR^{1b}₂, phenyl, —SR^{1b}, —OR^{1b}, and 3- to 12-membered heterocycle;
R² is selected from hydrogen, —OH, halogen, C₁₋₆ alkoxy, C₁₋₆ haloalkoxy, —NH₂, and C₁₋₆ alkylamino;
R³ is selected from optionally substituted C₁₋₆ alkyl, optionally substituted C₁₋₆ haloalkyl, —CO—, —NR^{1b}—, and —O—;
each X is independently selected at each occurrence from —OR^{1b}, halogen, C₁₋₆ alkyl, or C₁₋₆ haloalkyl; and
s is selected from 0, 1, 2, and 3;
wherein R^{1b} is hydrogen or C₁-C₆ alkyl.

In some embodiments, R¹ is —C(O)R^{1a}.

In some embodiments, R^{1a} is C₅-C₈ carbocycle. In some embodiments, R^{1a} is C₅ carbocycle or C₆ carbocycle. In some embodiments, R^{1a} is C₅ carbocycle. In some embodiments, R^{1a} is

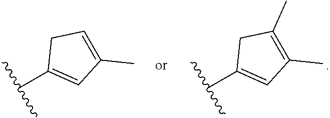

In some embodiments, R^{1a} is

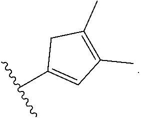 [SMILES: *C1=CC(C)=C(C)C1]

In some embodiments, R^{1a} is C₆ carbocycle. In some embodiments, R^{1a} is selected from

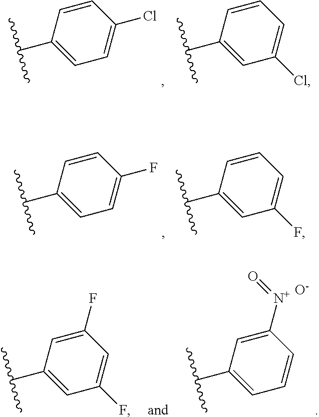

In some embodiments, R^{1a} is selected from

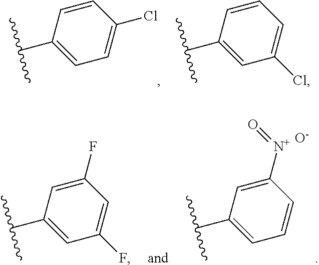

In some embodiments, R² is halogen. In some embodiments, R² is —Cl. In some embodiments, R² is —NH₂.

In some embodiments, R³ is optionally substituted C₁₋₆ alkyl. In some embodiments, R³ is C₁ alkyl. In some embodiments, R³ is —NR^{1b}—. In some embodiments, R³ is —NH—.

In some embodiments, X is —OR^{1b}. In some embodiments, X is —OH. In some embodiments, X is —OCH₃. In some embodiments, X is halogen. In some embodiments, X is —Cl.

In some embodiments, s is 1 or 2.

In some embodiments, the compound is selected from the group consisting of:

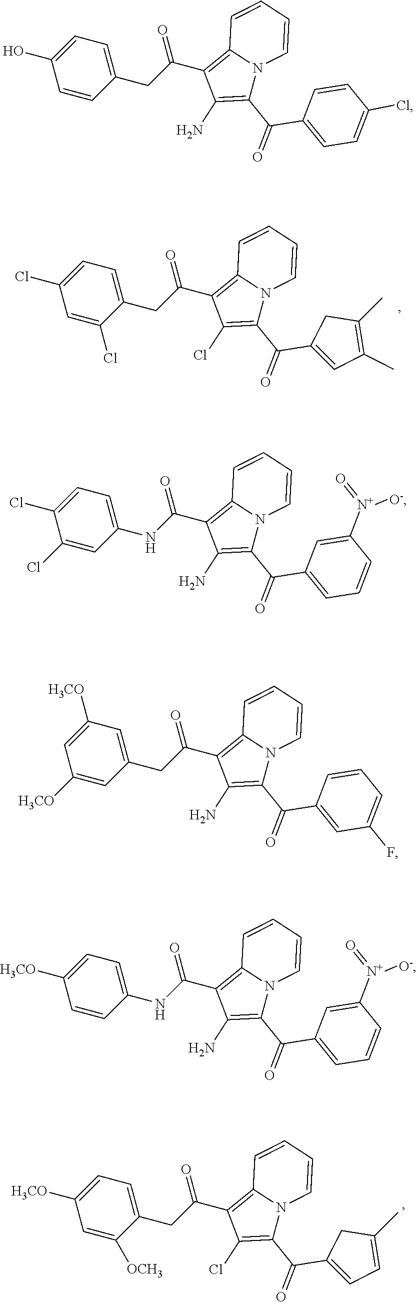

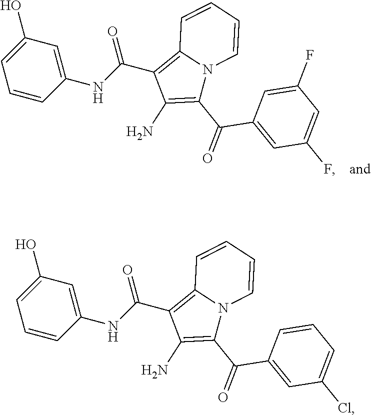 [SMILES: Nc1c(C(=O)Nc2cccc(O)c2)c2ccccn2c1C(=O)c1cc(F)cc(F)c1 ; "and" ; Nc1c(C(=O)Nc2cccc(O)c2)c2ccccn2c1C(=O)c1cccc(Cl)c1]

and a salt thereof.

In some embodiments, the disease, disorder, or condition comprises a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the disease, disorder, or condition comprises a peripheral immune/inflammatory condition. In some embodiments, the peripheral immune/inflammatory condition comprises atopic dermatitis, contact dermatitis, dyshidrotic eczema, neurodermatitis, nummular eczema, seborrheic dermatitis, or stasis dermatitis.

In some embodiments, the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

In some embodiments, the administration reduces dryness and redness of the skin, and drives a skin soothing effect. In some embodiments, the administration reduces itching, irritation, and cracking. In some embodiments, the administration decreases the expression of interleukin 4 (IL-4) or interleukin 13 (IL-13). In some embodiments, the administration decreases the expression of IL-4 in activated Th2 cells. In some embodiments, the administration decreases the expression of IL-13 in activated Th2 cells. In some embodiments, the administration blocks or reduces engagement between IL-4 and interleukin 4 receptor, alpha (IL-4Rα). In some embodiments, the administration blocks or reduces engagement between a receptor complex that is composed of interleukin 4 receptor, alpha (IL-4Rα), and an interacting cytokine (i.e., IL-4, IL-13, etc.).

In some embodiments, the composition further comprises an additive selected from the group consisting of pharmaceutically acceptable carriers, excipients, adjuvants, diluents, and combinations thereof. In some embodiments, the composition is formulated as a form of an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol. In some embodiments, the composition is formulated as an edible supplement, a beverage, or a jelly.

In some embodiments, the composition is formulated for administration by a topical application. In some embodiments, the composition is formulated for administration by a direct injection, an inhalational route, a transdermal route, an intranasal route, an oral route, a sublingual route, a buccal route, an intravenous injection, an intramuscular injection, a subcutaneous injection, a rectal route, or vaginal route.

The present disclosure provides a method for treating, preventing, or providing relief for a disease, disorder, or condition in a subject in need thereof, comprising administering the subject a composition comprising a compound having a structure of Formula (II) or salt thereof,

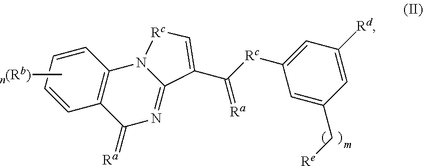

wherein
each R^{a} is independently selected from O, S, and NH;
each R^{b} is independently selected at each occurrence from —OR^{b1}, halogen, C₁₋₆ alkoxy, C₁₋₆ haloalkoxy, —NR^{b1}₂, and C₁₋₆ alkylamino;
each R^{c} is independently selected from O, S, or NH;
R^{d} is selected from hydrogen, —OR^{b1}, halogen, C₁₋₆ alkoxy, C₁₋₆ haloalkoxy, —NR^{b1}₂, and C₁₋₆ alkylamino;
R^{e} is selected from —C₁-C₆ alkyl, optionally substituted C₅-C₁₀ carbocycle, and optionally substituted C₅-C₁₀ heterocycle;
n is selected from 0, 1, 2, or 3; and
m is selected from 1, 2, or 3,
wherein R^{b1} is hydrogen or C₁-C₆ alkyl.

In some embodiments, R^{a} is O.

In some embodiments, R^{b} is —OR^{b1} or halogen. In some embodiments, R^{b} is —OR^{b1}. In some embodiments, R^{b} is —OH. In some embodiments, R^{b} is halogen. In some embodiments, R^{b} is —Cl.

In some embodiments, n is 0, 1, or 2.

In some embodiments, R^{c} is S or NH.

In some embodiments, R^{d} is selected from hydrogen, —OR^{b1}, and halogen. In some embodiments, R^{d} is hydrogen. In some embodiments, R^{d} is —OR^{b1}. In some embodiments, R^{d} is —OH. In some embodiments, R^{d} is halogen. In some embodiments, R^{d} is —Cl.

In some embodiments, R^{e} is optionally substituted C₅-C₁₀ heterocycle. In some embodiments, R^{e} is selected from the group consisting of:

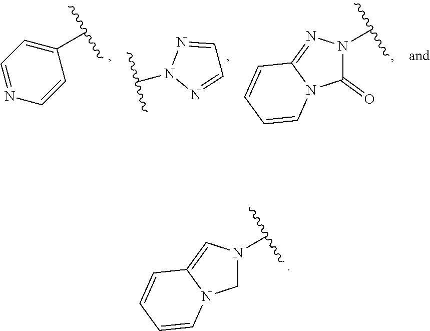

In some embodiments, m is 1 or 2.

In some embodiments, the compound is selected from the group consisting of:

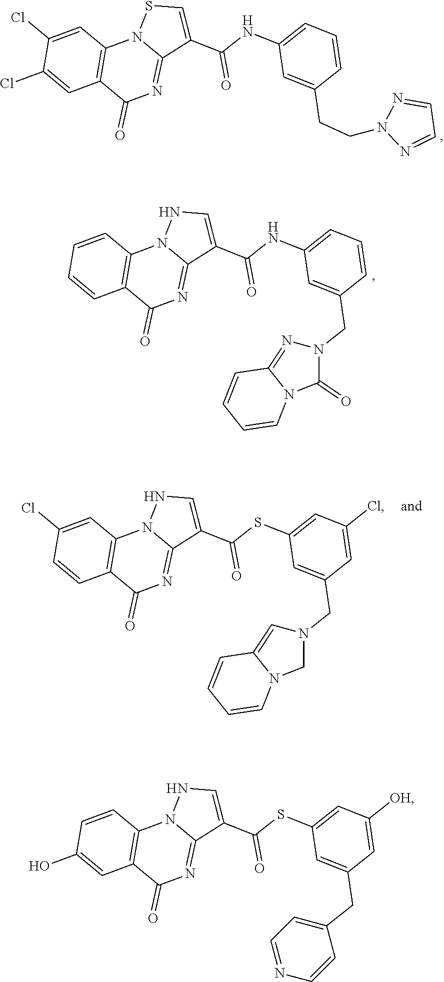 [SMILES: O=C(Nc1cccc(CCn2nccn2)c1)c1csn2c1nc(=O)c1cc(Cl)c(Cl)cc12 ; O=C(Nc1cccc(Cn2nc3ccccn3c2=O)c1)c1c[nH]n2c1nc(=O)c1ccccc12 ; O=C(Sc1cc(Cl)cc(CN2C=C3C=CC=CN3C2)c1)c1c[nH]n2c1nc(=O)c1ccc(Cl)cc12 ; O=C(Sc1cc(O)cc(Cc2ccncc2)c1)c1c[nH]n2c1nc(=O)c1cc(O)ccc12]

and a salt thereof.

In some embodiments, the disease, disorder, or condition comprises a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the disease, disorder, or condition comprises a peripheral immune/inflammatory condition. In some embodiments, the peripheral immune/inflammatory condition comprises atopic dermatitis, contact dermatitis, dyshidrotic eczema, neurodermatitis, nummular eczema, seborrheic dermatitis, or stasis dermatitis.

In some embodiments, the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

In some embodiments, the administration reduces dryness, itching, redness, roughness, or flakiness of skin. In some embodiments, the administration decreases the expression of interleukin 4 (IL-4) or interleukin 13 (IL-13). In some embodiments, the administration decreases the expression of IL-4 in activated Th2 cells. In some embodiments, the administration decreases the expression of IL-13 in activated Th2 cells. In some embodiments, the administration blocks or reduces engagement between IL-4 and interleukin 4 receptor, alpha (IL-4Rα). In some embodiments, the administration blocks or reduces engagement between a receptor complex that is composed of interleukin 4 receptor, alpha (IL-4Rα), and an interacting cytokine (i.e., IL-4, IL-13, etc.).

In some embodiments, the composition further comprises an additive selected from the group consisting of pharmaceutically acceptable carriers, excipients, adjuvants, diluents, and combinations thereof. In some embodiments, the composition is formulated as a form of an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol. In some embodiments, the composition is formulated as an edible supplement, a beverage, or a jelly.

In some embodiments, the composition is formulated for administration by a topical application. In some embodiments, the composition is formulated for administration by a direct injection, an inhalational route, a transdermal route, an intranasal route, an oral route, a sublingual route, a buccal route, an intravenous injection, an intramuscular injection, a subcutaneous injection, a rectal route, or vaginal route.

The present disclosure provides a method for treating, preventing, or providing a relief for a disease, disorder, or condition in a subject in need thereof, comprising administering the subject a composition comprising a compound having a structure of Formula (III) or salt thereof,

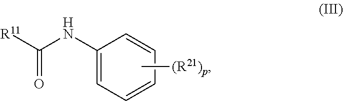

wherein
R¹¹ is selected from —CH₂R¹², —OR¹², —SR¹², —C(O)R¹², —C(O)OR¹², —OC(O)R¹², —OC(O)N(R¹²)₂, —NR¹²S(O)₂R¹², —C(O)N(R¹²)₂, —S(O)₂(R¹²), —S(O)₂N(R¹²)₂, optionally substituted C₃-C₁₂ carbocycle, and optionally substituted 5- to 14-membered heterocycle;
   each R¹² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle;
each R²¹ is independently selected at each occurrence from —OH, halogen, C₁₋₆ alkyl, C₁₋₆ haloalkyl, OR²², —SR²², —C(O)R²², —C(O)OR²², —OC(O)R²², —OCH₂C(O)R²², —OC(O)N(R²²)₂, —OCH₂C(O)NHR²², —OCH₂C(O)N(R²²)₂, —NR²²S(O)₂R²², —C(O)N(R²²)₂, —S(O)₂(R²²), —S(O)₂NHR²², —S(O)₂N(R²²)₂, —NHC(O)R²², optionally substituted C₃-C₁₂ carbocycle, and optionally substituted 5- to 14-membered heterocycle;
   each R²² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle; and
p is selected from 0, 1, 2, 3, or 4.

In some embodiments, R¹¹ is —CH₂R¹². In some embodiments, R¹¹ is —OR¹².

In some embodiments, R¹² is optionally substituted C₃-C₁₂ carbocycle or optionally substituted 5- to 14-membered heterocycle. In some embodiments, R¹² is selected from

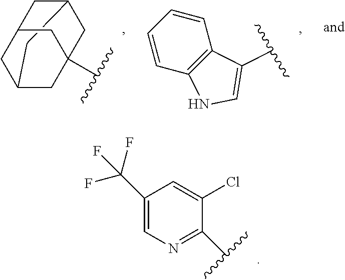 [SMILES: *C12CC3CC(CC(C3)C1)C2 ; *c1c[nH]c2ccccc12 ; *c1ncc(C(F)(F)F)cc1Cl]

In some embodiments, R¹¹ is optionally substituted C₃-C₁₂ carbocycle or optionally substituted 5- to 14-membered heterocycle. In some embodiments, R¹¹ is selected from

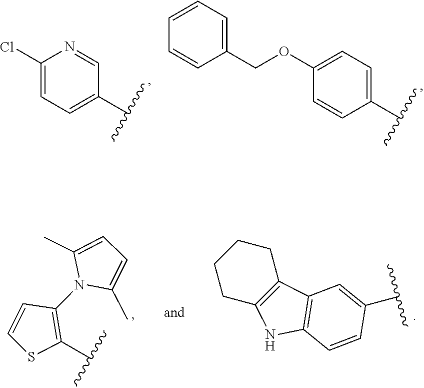 [SMILES: *c1ccc(Cl)nc1 ; *c1ccc(OCc2ccccc2)cc1 ; *c1sccc1-n1c(C)ccc1C ; *c1ccc2[nH]c3c(c2c1)CCCC3]

In some embodiments, R²¹ is —OH. In some embodiments, R²¹ is C₁₋₆ alkyl. In some embodiments, R²¹ is C₁ alkyl. In some embodiments, R²¹ is —OCH₂C(O)NHR²², wherein R²² is C₂ alkyl. In some embodiments, R²¹ is —S(O)₂NHR²², wherein R²² is 5- to 14-membered heterocycle. In some embodiments, R²¹ is —NHC(O)R²², wherein R²² is C₃-C₁₂ carbocycle. In some embodiments, R²¹ is optionally substituted C₃-C₁₂ carbocycle or optionally substituted 5- to 14-membered heterocycle.

In some embodiments, R²² is selected from —CH₂CH₃,

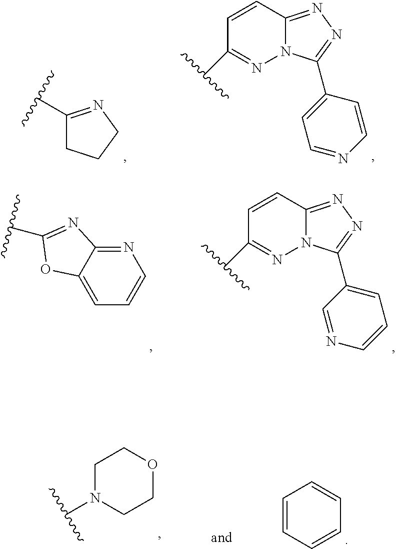

In some embodiments, R²¹ is selected from —OH, —CH₃,

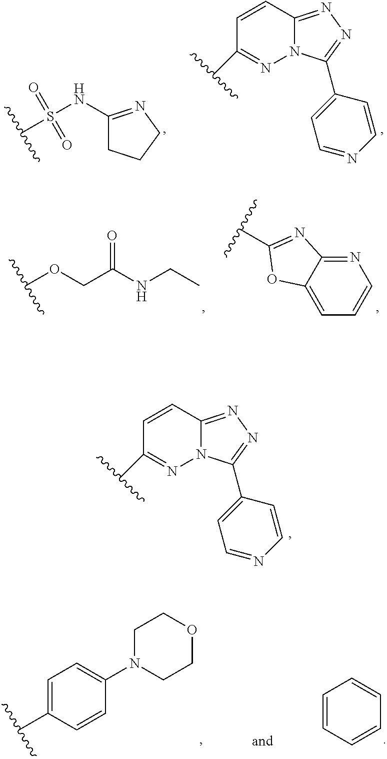

In some embodiments, p is selected from 1, 2, and 3.

In some embodiments, the compound is selected from the group consisting of:

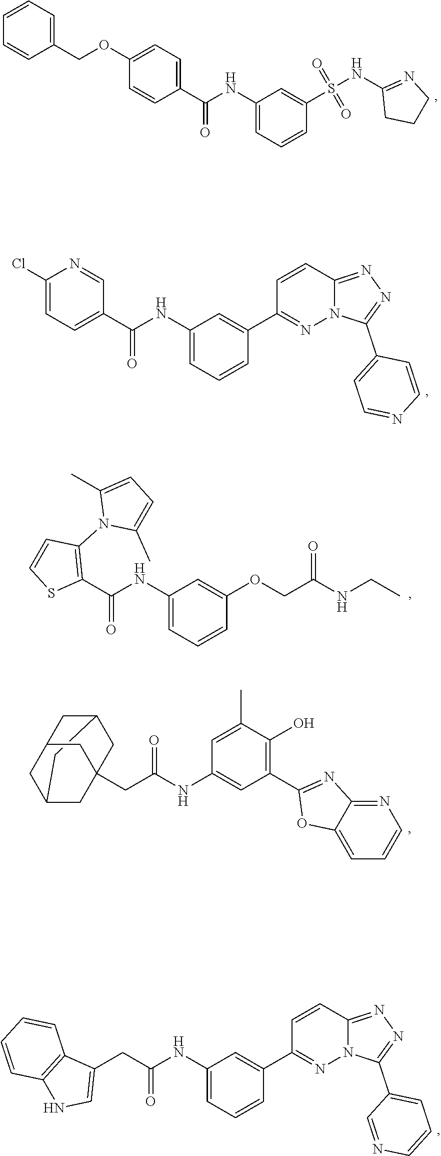 [SMILES: O=C(Nc1cccc(S(=O)(=O)NC2=NCCC2)c1)c1ccc(OCc2ccccc2)cc1 ; O=C(Nc1cccc(-c2ccc3nnc(-c4ccncc4)n3n2)c1)c1ccc(Cl)nc1 ; CCNC(=O)COc1cccc(NC(=O)c2sccc2-n2c(C)ccc2C)c1 ; Cc1cc(NC(=O)CC23CC4CC(CC(C4)C2)C3)cc(-c2nc3ncccc3o2)c1O ; O=C(Cc1c[nH]c2ccccc12)Nc1cccc(-c2ccc3nnc(-c4cccnc4)n3n2)c1]

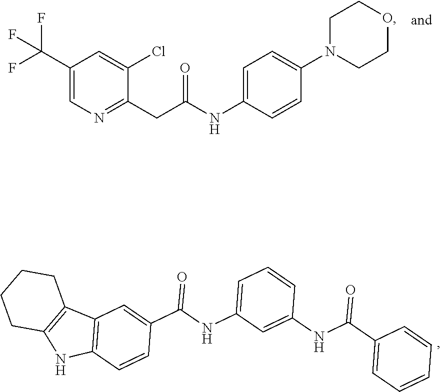 [SMILES: O=C(Cc1ncc(C(F)(F)F)cc1Cl)Nc1ccc(N2CCOCC2)cc1 ; O=C(Nc1cccc(NC(=O)c2ccc3[nH]c4c(c3c2)CCCC4)c1)c1ccccc1]

and a salt thereof.

In some embodiments, the disease, disorder, or condition comprises a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the disease, disorder, or condition comprises a peripheral immune/inflammatory condition. In some embodiments, the peripheral immune/inflammatory condition comprises atopic dermatitis, contact dermatitis, dyshidrotic eczema, neurodermatitis, nummular eczema, seborrheic dermatitis, or stasis dermatitis.

In some embodiments, the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

In some embodiments, the administration reduces dryness & redness of the skin, and drives a skin soothing effect. In some embodiments, the administration reduces itching, irritation, and cracking. In some embodiments, the administration decreases the expression of interleukin 4 (IL-4) or interleukin 13 (IL-13). In some embodiments, the administration decreases the expression of IL-4 in activated Th2 cells. In some embodiments, the administration decreases the expression of IL-13 in activated Th2 cells. In some embodiments, the administration blocks or reduces engagement between IL-4 and interleukin 4 receptor, alpha (IL-4Rα). In some embodiments, the administration blocks or reduces engagement between a receptor complex that is composed of interleukin 4 receptor, alpha (IL-4Rα), and an interacting cytokine (i.e., IL-4, IL-13, etc.).

In some embodiments, the composition further comprises an additive selected from the group consisting of pharmaceutically acceptable carriers, excipients, adjuvants, diluents, and combinations thereof. In some embodiments, the composition is formulated as a form of an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol. In some embodiments, the composition is formulated as an edible supplement, a beverage, or a jelly.

In some embodiments, the composition is formulated for administration by a topical application. In some embodiments, the composition is formulated for administration by a direct injection, an inhalational route, a transdermal route, an intranasal route, an oral route, a sublingual route, a buccal route, an intravenous injection, an intramuscular injection, a subcutaneous injection, a rectal route, or vaginal route.

The present disclosure provides a method for treating, preventing, or providing a relief for a disease, disorder, or condition in a subject in need thereof, comprising administering the subject a composition comprising a compound having a structure of Formula (IV) or salt thereof,

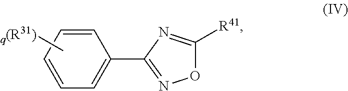

wherein
each R³¹ is independently selected at each occurrence from halogen, —OR^{3a}, —CN, —NO₂, —NR^{3a}₂, C₁₋₁₀ alkyl, —C₁₋₁₀ haloalkyl, —O—C₁₋₁₀ alkyl, C₂₋₁₀ alkenyl, C₂₋₁₀ alkynyl, optionally substituted C₃₋₁₂ carbocycle, and optionally substituted 3- to 12-membered heterocycle, wherein R^{3a} is hydrogen or C₁-C₆ alkyl;
q is selected from 0, 1, or 2;
R⁴¹ is selected from hydrogen, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents selected from ═O, phenyl, —C(O)R⁴², wherein R⁴² is selected from —OH, —CN, —NO₂, —NH₂, C₁₋₁₀ alkyl, optionally substituted C₃₋₁₂ carbocycle, and optionally substituted 3- to 12-membered heterocycle.

In some embodiments, R³¹ is halogen. In some embodiments, R³¹ is —Cl.

In some embodiments, R³¹ is optionally substituted 3- to 12-membered heterocycle. In some embodiments, R³¹ is optionally substituted 5-membered heterocycle. In some embodiments, R³¹ is selected from a triazole, wherein the triazole is optionally substituted with one or more R³².

In some embodiments, each R³² is independently selected at each occurrence from halogen, C₁₋₆ alkyl, C₂₋₆ alkenyl, and C₂₋₆ alkynyl. In some embodiments, each R³² is C₁ alkyl. In some embodiments, R³¹ is

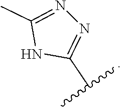 [SMILES: *c1nnc(C)[nH]1]

In some embodiments, q is 0 or 1.

In some embodiments, R⁴¹ is optionally substituted 3- to 12-membered heterocycle. In some embodiments, R⁴¹ is selected from

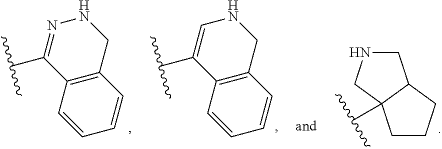 [SMILES: *C1=NNCc2ccccc21 ; *C1=CNCc2ccccc21 ; *C12CCCC1CNC2]

In some embodiments, R⁴² is optionally substituted 3- to 12-membered heterocycle. In some embodiments, R⁴² is optionally substituted 9-membered heterocycle. In some embodiments, R⁴² is

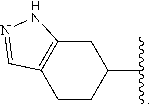 [SMILES: *C1CCc2cn[nH]c2C1]

In some embodiments, the compound is selected from the group consisting of:

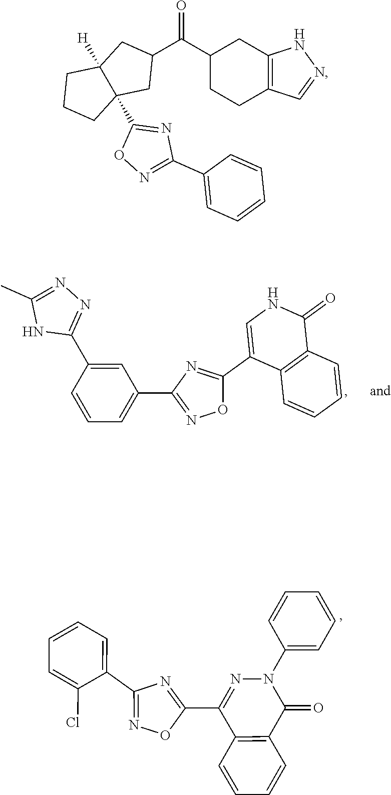 [SMILES: O=C(C1CCc2cn[nH]c2C1)C1C[C@@H]2CCC[C@]2(c2nc(-c3ccccc3)no2)C1 ; Cc1nnc(-c2cccc(-c3noc(-c4c[nH]c(=O)c5ccccc45)n3)c2)[nH]1 ; O=c1c2ccccc2c(-c2nc(-c3ccccc3Cl)no2)nn1-c1ccccc1]

and a salt thereof.

In some embodiments, the disease, disorder, or condition comprises a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the disease, disorder, or condition comprises a peripheral immune/inflammatory condition. In some embodiments, the peripheral immune/inflammatory condition comprises atopic dermatitis, contact dermatitis, dyshidrotic eczema, neurodermatitis, nummular eczema, seborrheic dermatitis, or stasis dermatitis.

In some embodiments, the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

In some embodiments, the administration reduces dryness and redness of the skin, and drives a skin soothing effect. In some embodiments, the administration reduces itching, irritation, and cracking. In some embodiments, the administration decreases the expression of interleukin 4 (IL-4) or interleukin 13 (IL-13). In some embodiments, the administration decreases the expression of IL-4 in activated Th2 cells. In some embodiments, the administration decreases the expression of IL-13 in activated Th2 cells. In some embodiments, the administration blocks or reduces engagement between IL-4 and interleukin 4 receptor, alpha (IL-4Rα). In some embodiments, the administration blocks or reduces engagement between a receptor complex that is composed of interleukin 4 receptor, alpha (IL-4Rα), and an interacting cytokine (i.e., IL-4, IL-13, etc.).

In some embodiments, the composition further comprises an additive selected from the group consisting of pharmaceutically acceptable carriers, excipients, adjuvants, diluents, and combinations thereof. In some embodiments, the composition is formulated as a form of an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol. In some embodiments, the composition is formulated as an edible supplement, a beverage, or a jelly.

In some embodiments, the composition is formulated for administration by a topical application. In some embodiments, the composition is formulated for administration by a direct injection, an inhalational route, a transdermal route, an intranasal route, an oral route, a sublingual route, a buccal route, an intravenous injection, an intramuscular injection, a subcutaneous injection, a rectal route, or vaginal route.

The present disclosure provides a method for treating, preventing, or providing a relief for a disease, disorder, or condition in a subject in need thereof, comprising administering the subject a composition comprising a compound having a structure of Formula (V) or salt thereof,

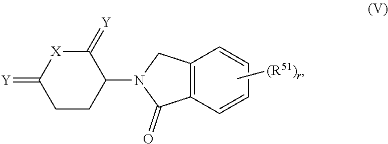

wherein
X is selected from O, S, and NH;
Y is selected from O, S, and NH;
each R⁵¹ is independently selected at each occurrence from halogen, —OR⁵², —CN, —NO₂, —NR⁵²₂, C₁₋₁₀ alkyl, —C₁₋₁₀ haloalkyl, —OC(O)R⁵², —OCH₂C(O)R⁵², C₃₋₁₂ carbocycle, and optionally substituted 3- to 12-membered heterocycle;
   R⁵² is selected from hydrogen, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents selected from ═O, phenyl, —OH and optionally substituted 3- to 12-membered heterocycle; and
r is selected from 0, 1, 2, or 3.

In some embodiments, X is S or NH. In some embodiments, Y is O.

In some embodiments, each R⁵¹ is independently selected at each occurrence from —OR⁵², —OCH₂C(O)R⁵², and optionally substituted 3- to 12-membered heterocycle. In some embodiments, R⁵¹ is —OR⁵². In some embodiments, R⁵² is —C₁₋₆ alkyl. In some embodiments, R⁵¹ is —OCH₂C(O)R⁵². In some embodiments, R⁵² is optionally substituted 3- to 12-membered heterocycle. In some embodiments, R⁵² is optionally substituted 6-membered heterocycle. In some embodiments, R⁵² is selected from

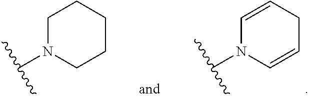 [SMILES: *N1CCCCC1 ; *N1C=CCC=C1]

In some embodiments, R⁵² is optionally substituted with one substituent selected from —OH and 3- to 12-membered heterocycle. In some embodiments, R⁵² is selected from

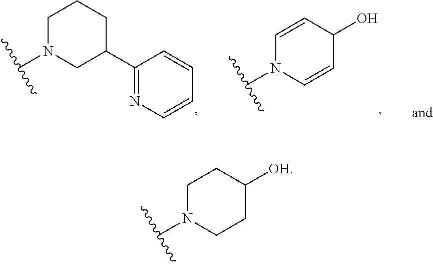 [SMILES: *N1CCCC(c2ccccn2)C1 ; *N1C=CC(O)C=C1 ; *N1CCC(O)CC1]

In some embodiments, R⁵¹ is optionally substituted 3- to 12-membered heterocycle. In some embodiments, R⁵¹ is optionally substituted 6-membered heterocycle. In some embodiments, R⁵¹ is selected from optionally substituted pyridine, tetrahydropyran, pyridazine, pyrimidine, and pyrazine. In some embodiments, R⁵¹ is selected from optionally substituted pyridine. In some embodiments, R⁵¹ is

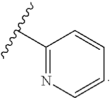 [SMILES: *c1ccccn1]

In some embodiments, r is 1 or 2.

In some embodiments, the compound is selected from the group consisting of:

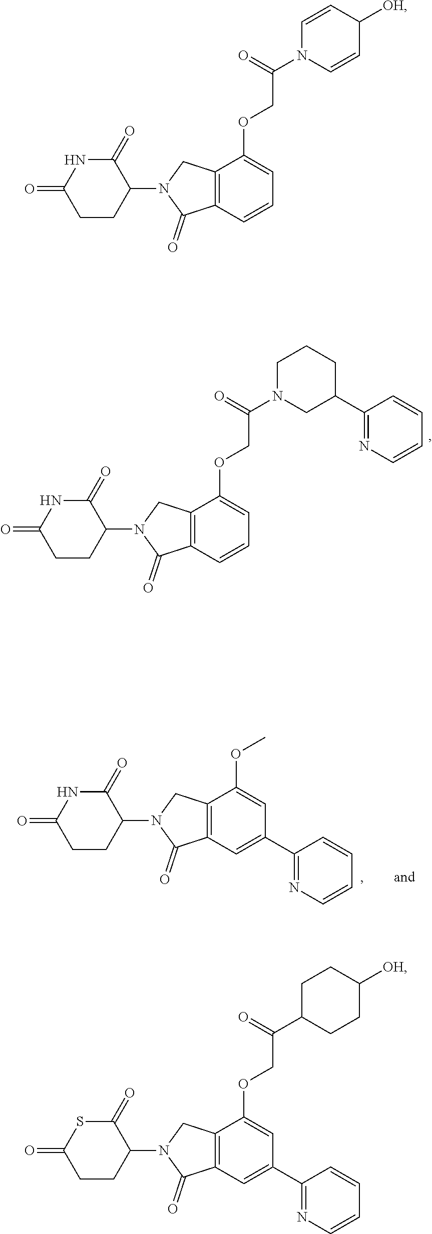 [SMILES: O=C1CCC(N2Cc3c(OCC(=O)N4C=CC(O)C=C4)cccc3C2=O)C(=O)N1 ; O=C1CCC(N2Cc3c(OCC(=O)N4CCCC(c5ccccn5)C4)cccc3C2=O)C(=O)N1 ; COc1cc(-c2ccccn2)cc2c1CN(C1CCC(=O)NC1=O)C2=O ; O=C1CCC(N2Cc3c(OCC(=O)C4CCC(O)CC4)cc(-c4ccccn4)cc3C2=O)C(=O)S1]

and a salt thereof.

In some embodiments, the disease, disorder, or condition comprises a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the disease, disorder, or condition comprises a peripheral immune/inflammatory condition. In some embodiments, the peripheral immune/inflammatory condition comprises atopic dermatitis, contact dermatitis, dyshidrotic eczema, neurodermatitis, nummular eczema, seborrheic dermatitis, or stasis dermatitis.

In some embodiments, the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

In some embodiments, the administration reduces dryness and redness of the skin, and drives a skin soothing effect. In some embodiments, the administration reduces itching, irritation, and cracking. In some embodiments, the administration decreases the expression of interleukin 4 (IL-4) or interleukin 13 (IL-13). In some embodiments, the administration decreases the expression of IL-4 in activated Th2 cells. In some embodiments, the administration decreases the expression of IL-13 in activated Th2 cells. In some embodiments, the administration blocks or reduces engagement between IL-4 and interleukin 4 receptor, alpha (IL-4Rα). In some embodiments, the administration blocks or reduces engagement between a receptor complex that is composed of interleukin 4 receptor, alpha (IL-4Rα), and an interacting cytokine (i.e., IL-4, IL-13, etc.).

In some embodiments, the composition further comprises an additive selected from the group consisting of pharmaceutically acceptable carriers, excipients, adjuvants, diluents, and combinations thereof. In some embodiments, the composition is formulated as a form of an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol. In some embodiments, the composition is formulated as an edible supplement, a beverage, or a jelly.

In some embodiments, the composition is formulated for administration by a topical application. In some embodiments, the composition is formulated for administration by a direct injection, an inhalational route, a transdermal route, an intranasal route, an oral route, a sublingual route, a buccal route, an intravenous injection, an intramuscular injection, a subcutaneous injection, a rectal route, or vaginal route.

The present disclosure provides a method for treating, preventing, or providing a relief for a disease, disorder, or condition in a subject in need thereof, comprising administering the subject a composition comprising a compound having a structure selected from the group consisting of:

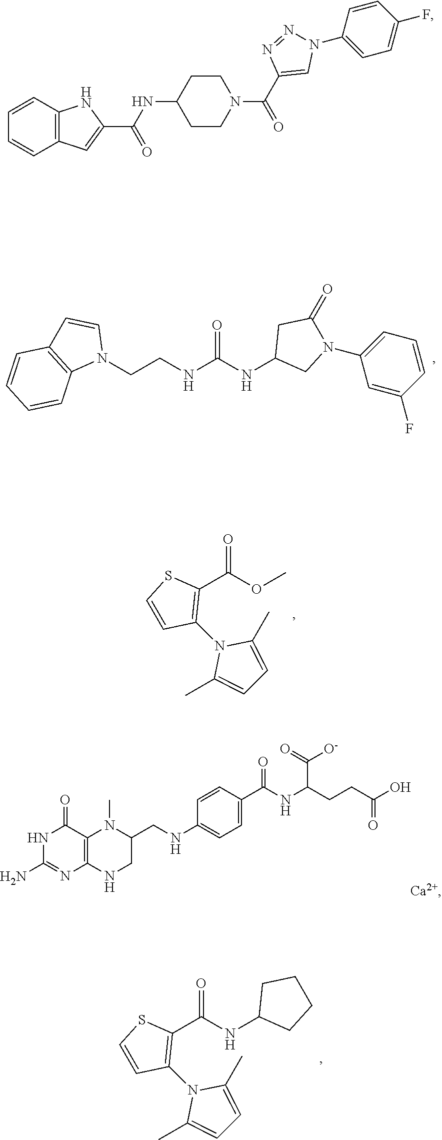 [SMILES: O=C(NC1CCN(C(=O)c2cn(-c3ccc(F)cc3)nn2)CC1)c1cc2ccccc2[nH]1 ; O=C(NCCn1ccc2ccccc21)NC1CC(=O)N(c2cccc(F)c2)C1 ; COC(=O)c1sccc1-n1c(C)ccc1C ; CN1c2c(nc(N)[nH]c2=O)NCC1CNc1ccc(C(=O)NC(CCC(=O)O)C(=O)[O-])cc1 ; Cc1ccc(C)n1-c1ccsc1C(=O)NC1CCCC1]

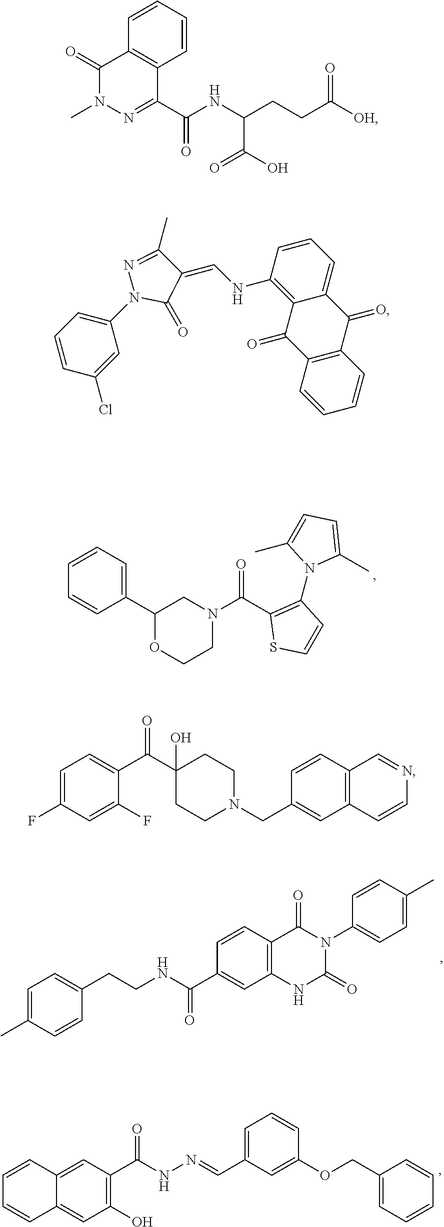

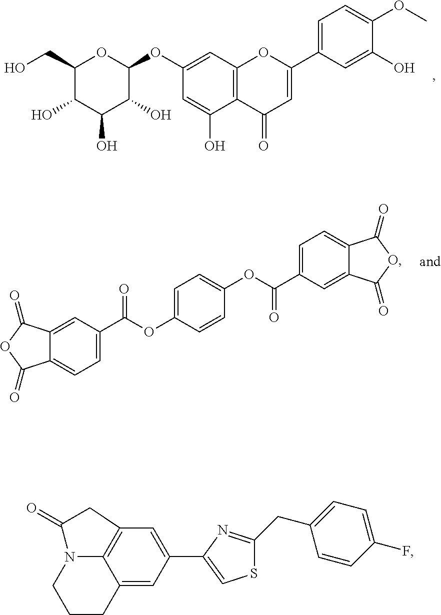 [SMILES: COc1ccc(-c2cc(=O)c3c(O)cc(O[C@@H]4O[C@H](CO)[C@@H](O)[C@H](O)[C@H]4O)cc3o2)cc1O ; O=C(Oc1ccc(OC(=O)c2ccc3c(c2)C(=O)OC3=O)cc1)c1ccc2c(c1)C(=O)OC2=O ; O=C1Cc2cc(-c3csc(Cc4ccc(F)cc4)n3)cc3c2N1CCC3]

and a salt thereof.

In some embodiments, the disease, disorder, or condition comprises a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the disease, disorder, or condition comprises a peripheral immune/inflammatory condition. In some embodiments, the peripheral immune/inflammatory condition comprises atopic dermatitis, contact dermatitis, dyshidrotic eczema, neurodermatitis, nummular eczema, seborrheic dermatitis, or stasis dermatitis.

In some embodiments, the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

In some embodiments, the administration reduces dryness & redness of the skin, and drives a skin soothing effect. In some embodiments, the administration reduces itching, irritation, and cracking. In some embodiments, the administration decreases the expression of interleukin 4 (IL-4) or interleukin 13 (IL-13). In some embodiments, the administration decreases the expression of IL-4 in activated Th2 cells. In some embodiments, the administration decreases the expression of IL-13 in activated Th2 cells. In some embodiments, the administration blocks or reduces engagement between IL-4 and interleukin 4 receptor, alpha (IL-4Rα). In some embodiments, the administration blocks or reduces engagement between a receptor complex that is composed of interleukin 4 receptor, alpha (IL-4Rα), and an interacting cytokine (i.e., IL-4, IL-13, etc.).

In some embodiments, the composition further comprises an additive selected from the group consisting of pharmaceutically acceptable carriers, excipients, adjuvants, diluents, and combinations thereof. In some embodiments, the composition is formulated as a form of an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol. In some embodiments, the composition is formulated as an edible supplement, a beverage, or a jelly.

In some embodiments, the composition is formulated for administration by a topical application. In some embodiments, the composition is formulated for administration by a direct injection, an inhalational route, a transdermal route, an intranasal route, an oral route, a sublingual route, a buccal route, an intravenous injection, an intramuscular injection, a subcutaneous injection, a rectal route, or vaginal route.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “Figure” and “FIG.” herein), of which:

FIG. 1 shows the experimental process, in accordance with aspects of the present disclosure.

FIG. 2A shows normalized reduction in Interleukins-4 (IL-4) and Interleukins-13 (IL-13) among CD4+ Th2 cells. The normalized reduction was calculated from baseline, vehicle-treated CD4+ Th2 cells and each square represents a Tier 1 compound, in accordance with aspects of the present disclosure.

FIG. 2B shows % of IL-4 expression in CD4+ Th2 cells following treatment with II-2, in accordance with aspects of the present disclosure.

FIG. 2C shows representative histograms demonstrating change in IL-13 expression among CD4+ Th2 cells following treatment with compounds disclosed herein, in accordance with aspects of the present disclosure.

FIG. 2D shows representative histograms demonstrating change in IL-4 expression among CD4+ Th2 cells following treatment with the compounds disclosed herein, in accordance with aspects of the present disclosure.

FIG. 3A shows viability of various skin cells following treatment with the compounds disclosed herein after 48 hrs of treatment, in accordance with aspects of the present disclosure.

FIG. 3B shows normalized activation of the Nrf2-ARE pathway following 24 hrs. Cinnamic aldehyde was used as the positive control, in accordance with aspects of the present disclosure.

FIG. 3C shows normalized caspase-3/7 activation following treatment with the compounds disclosed herein after 24 hrs. ABT-263, a known senolytic, was used as a positive control. Data represents mean+/−s.d. and is representative of at least two experimental replicates, in accordance with aspects of the present disclosure.

FIG. 4A-FIG. 4C show results of IL-4 signaling inhibition assays using engineered HEK-Blue™ IL-4/IL-13 cells that were stimulated with IL-4 and co-cultured with the compounds disclosed herein, in accordance with aspects of the present disclosure.

FIG. 5 shows results of blood cell toxicity assay using peripheral blood mononuclear cells, in accordance with aspects of the present disclosure.

FIG. 6 shows inability of the compounds disclosed herein to activate monocyte-derived dendritic cells (moDCs), in accordance with aspects of the present disclosure.

FIG. 7 shows results of genotoxic assay of the compounds disclosed herein, in accordance with aspects of the present disclosure.

FIG. 8 shows evaluation of inducing oxidative stress of the compounds disclosed herein, in accordance with aspects of the present disclosure.

### DETAILED DESCRIPTION OF THE INVENTION

Interleukins-4 (IL-4) and Interleukins-13 (IL-13) are prominent cytokines playing substantial roles in immune system responses. They may function by associating with a receptor complex that includes interleukin-4 receptor alpha (IL-4Rα), situated on the cellular surface. These cytokines are integral to inflammatory responses, key for the body's defense against infections. However, overactivity or insufficient regulation of these cytokines can incite chronic inflammatory conditions, especially those affecting the skin. Conditions like atopic dermatitis (AD) are frequently linked to elevated levels of IL-4 and IL-13. Such states are characterized by symptoms like redness, dryness, itchiness, and heightened sensitivity. There's a need for a therapeutic solution that effectively manages these conditions by modulating the interactions of IL-4 or IL-13 with IL-4Rα.

Type 2-immunity includes typical adaptive responses to allergen and environmental exposure in atopic individuals. It may involve T Helper 2 (Th2) cells and immunoglobulin E. In atopic individuals, allergen and environmental exposure may promote activation of multiple Th2 cytokines, including interleukin (IL)-4, IL-5, IL-9, and IL-13, which amplify type-2 response. In particular, IL-4 and IL-13 may be central to pathogenesis and some therapeutic targets. Type 2-inflammation also characterizes other atopic disorders, such as food allergies, asthma, allergic rhinitis and conjunctivitis, which may be strongly associated with atopic dermatitis (AD). In some embodiments, the environmental factors include but are not limited to airborne allergens such as pollens, fungi, dust mites, or animal dander.

In certain aspects, the present disclosure provides an approach to manage inflammatory skin conditions. By inhibiting or reducing the interaction of IL-4 and IL-13 with IL-4Rα, inflammation is curtailed, and symptoms such as redness and dryness can improve, offering significant relief to patients. In certain aspects, the present disclosure provides compounds and methods for achieving desired cosmetic benefits of skin soothing and redness reduction. In certain aspects, the present disclosure provides compounds and methods useful for delivering skin soothing, redness relief, and reduction in skin irritation, itching, cracking, peeling, and inflammation, alone or in combination with colloidal oatmeal in a subject in need thereof. In some embodiments, the present disclosure provides compounds and methods useful for preventing or treating a disease, disorder, or condition of a subject in need thereof. In some embodiments, the present disclosure provides compounds and methods useful for treating a disease, disorder, or condition of a subject in need thereof. In some embodiments, the present disclosure provides compounds and methods useful for providing a relief of a disease, disorder, or condition of a subject in need thereof. In some embodiments, the disease, disorder, or condition comprises a disease, disorder, or condition of skin comprising eczema (e.g. whether or not a diagnosis has been made). In some embodiments, the disease, disorder, or condition comprises a peripheral immune/inflammatory condition. In some embodiments, the compound may have a chemical structure of Formula (I), (II), (III), (IV), (V), or a Table 6, or a salt thereof. In some embodiments, the compound may be included in a pharmaceutical or cosmetic composition or formulation. In some embodiments, the compounds disclosed herein decrease the expression of IL-4 or IL-13 in activated Th2 cells. In some embodiments, the compounds disclosed herein decrease the expression of IL-4 in activated Th2 cells. In some embodiments, the compounds disclosed herein decrease the expression of IL-13 in activated Th2 cells. In some embodiments, the compounds disclosed herein block or reduce engagement between IL-4 and interleukin 4 receptor, alpha (IL-4Rα). In some embodiments, the compounds disclosed herein reduce engagement between a receptor complex that is composed of interleukin 4 receptor, alpha (IL-4Rα), and an interacting cytokine (ie: IL-4, IL-13, etc.).

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The expressions “at least one of A and B” and “at least one of A or B” may be construed to mean at least A, at least B, or at least A and B (i.e., a set comprising A and B, which set may include one or more additional elements). The term “A and/or B” may be construed to mean only A, only B, or both A and B.

The expressions “at least about A, B, and C” and “at least about A, B, or C” may be construed to mean at least about A, at least about B, or at least about C. The expressions “at most about A, B, and C” and “at most about A, B, or C” may be construed to mean at most about A, at most about B, or at most about C.

The expression “between about A and B, C and D, and E and F” may be construed to mean between about A and about B, between about C and about D, and between about E and about F. The expression “between about A and B, C and D, or E and F” may be construed to mean between about A and about B, between about C and about D, or between about E and about F.

The expression “about A to B and C to D” may be construed to mean between about A and about B and between about C and about D. The expression “about A to B or C to D” may be construed to mean between about A and about B or between about C and about D.

The term “exemplary” as used herein means “serving as an example, instance, or illustration.” Any embodiment described herein as “exemplary” is not to be construed as preferred or advantageous over other embodiments.

“Alkyl” refers to a straight or branched hydrocarbon chain radical consisting solely of carbon and hydrogen atoms, containing no unsaturation, and preferably having from one to fifteen carbon atoms (i.e., C₁-C₁₅ alkyl). In certain embodiments, an alkyl comprises one to thirteen carbon atoms (i.e., C₁-C₁₃ alkyl). In certain embodiments, an alkyl comprises one to eight carbon atoms (i.e., C₁-C₈ alkyl). In other embodiments, an alkyl comprises one to five carbon atoms (i.e., C₁-C₅ alkyl). In other embodiments, an alkyl comprises one to four carbon atoms (i.e., C₁-C₄ alkyl). In other embodiments, an alkyl comprises one to three carbon atoms (i.e., C₁-C₃ alkyl). In other embodiments, an alkyl comprises one to two carbon atoms (i.e., C₁-C₂ alkyl). In other embodiments, an alkyl comprises one carbon atom (i.e., C₁ alkyl). In other embodiments, an alkyl comprises five to fifteen carbon atoms (i.e., C₅-C₁₅ alkyl). In other embodiments, an alkyl comprises five to eight carbon atoms (i.e., C₅-C₈ alkyl). In other embodiments, an alkyl comprises two to five carbon atoms (i.e., C₂-C₅ alkyl). In other embodiments, an alkyl comprises three to five carbon atoms (i.e., C₃-C₅ alkyl). In certain embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

The term “C_{x-y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons. The term —C_{x-y}alkylene- refers to a substituted or unsubstituted alkylene chain with from x to y carbons in the alkylene chain. For example —C₁₋₆alkylene- may be selected from methylene, ethylene, propylene, butylene, pentylene, and hexylene, any one of which is optionally substituted.

The terms “C_{x-y}” and “Cₓ-C_{y}” are used herein interchangeably. The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” or “C₁-C₆alkyl” refers to substituted or unsubstituted saturated hydrocarbon groups, including straight-chain alkyl and branched-chain alkyl groups that contain from 1 to 6 carbons. The term —C_{x-y}alkylene- or —Cₓ-C_{y}alkylene- refers to a substituted or unsubstituted alkylene chain with from x to y carbons in the alkylene chain. For example —C₁₋₆alkylene- or —C₁-C₆alkylene- may be selected from methylene, ethylene, propylene, butylene, pentylene, and hexylene, any one of which is optionally substituted.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and preferably having from two to twelve carbon atoms (i.e., C₂-C₁₂ alkenyl). In certain embodiments, an alkenyl comprises two to eight carbon atoms (i.e., C₂-C₈ alkenyl). In certain embodiments, an alkenyl comprises two to six carbon atoms (i.e., C₂-C₆ alkenyl). In other embodiments, an alkenyl comprises two to four carbon atoms (i.e., C₂-C₄ alkenyl). The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, and preferably having from two to twelve carbon atoms (i.e., C₂-C₁₂ alkynyl). In certain embodiments, an alkynyl comprises two to eight carbon atoms (i.e., C₂-C₈ alkynyl). In other embodiments, an alkynyl comprises two to six carbon atoms (i.e., C₂-C₆ alkynyl). In other embodiments, an alkynyl comprises two to four carbon atoms (i.e., C₂-C₄ alkynyl). The alkynyl is attached to the rest of the molecule by a single bond, for example, ethynyl, propynyl, butynyl, pentynyl, hexynyl, and the like.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively. The term —C_{x-y}alkenylene- refers to a substituted or unsubstituted alkenylene chain with from x to y carbons in the alkenylene chain. For example, —C₂₋₆alkenylene- may be selected from ethenylene, propenylene, butenylene, pentenylene, and hexenylene, any one of which is optionally substituted. An alkenylene chain may have one double bond or more than one double bond in the alkenylene chain. The term —C_{x-y}alkynylene- refers to a substituted or unsubstituted alkynylene chain with from x to y carbons in the alkenylene chain. For example, —C₂₋₆alkenylene- may be selected from ethynylene, propynylene, butynylene, pentynylene, and hexynylene, any one of which is optionally substituted. An alkynylene chain may have one triple bond or more than one triple bond in the alkynylene chain.

“Alkylene” or “alkylene chain” refers to a straight or branched divalent hydrocarbon chain linking the rest of the molecule to a radical group, consisting solely of carbon and hydrogen, containing no unsaturation, and preferably having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group may be through any two carbons within the chain. In certain embodiments, an alkylene comprises one to ten carbon atoms (i.e., C₁-C₈ alkylene). In certain embodiments, an alkylene comprises one to eight carbon atoms (i.e., C₁-C₈ alkylene). In other embodiments, an alkylene comprises one to five carbon atoms (i.e., C₁-C₅ alkylene). In other embodiments, an alkylene comprises one to four carbon atoms (i.e., C₁-C₄ alkylene). In other embodiments, an alkylene comprises one to three carbon atoms (i.e., C₁-C₃ alkylene). In other embodiments, an alkylene comprises one to two carbon atoms (i.e., C₁-C₂ alkylene). In other embodiments, an alkylene comprises one carbon atom (i.e., C₁ alkylene). In other embodiments, an alkylene comprises five to eight carbon atoms (i.e., C₅-C₈ alkylene). In other embodiments, an alkylene comprises two to five carbon atoms (i.e., C₂-C₅ alkylene). In other embodiments, an alkylene comprises three to five carbon atoms (i.e., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and preferably having from two to twelve carbon atoms. The alkenylene chain is attached to the rest of the molecule through a single bond and to the radical group through a single bond. The points of attachment of the alkenylene chain to the rest of the molecule and to the radical group may be through any two carbons within the chain. In certain embodiments, an alkenylene comprises two to ten carbon atoms (i.e., C₂-C₁₀ alkenylene). In certain embodiments, an alkenylene comprises two to eight carbon atoms (i.e., C₂-C₈ alkenylene). In other embodiments, an alkenylene comprises two to five carbon atoms (i.e., C₂-C₅ alkenylene). In other embodiments, an alkenylene comprises two to four carbon atoms (i.e., C₂-C₄ alkenylene). In other embodiments, an alkenylene comprises two to three carbon atoms (i.e., C₂-C₃ alkenylene). In other embodiments, an alkenylene comprises two carbon atom (i.e., C₂ alkenylene). In other embodiments, an alkenylene comprises five to eight carbon atoms (i.e., C₅-C₈ alkenylene). In other embodiments, an alkenylene comprises three to five carbon atoms (i.e., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and preferably having from two to twelve carbon atoms. The alkynylene chain is attached to the rest of the molecule through a single bond and to the radical group through a single bond. The points of attachment of the alkynylene chain to the rest of the molecule and to the radical group may be through any two carbons within the chain. In certain embodiments, an alkynylene comprises two to ten carbon atoms (i.e., C₂-C₁₀ alkynylene). In certain embodiments, an alkynylene comprises two to eight carbon atoms (i.e., C₂-C₈ alkynylene). In other embodiments, an alkynylene comprises two to five carbon atoms (i.e., C₂-C₅ alkynylene). In other embodiments, an alkynylene comprises two to four carbon atoms (i.e., C₂-C₄ alkynylene). In other embodiments, an alkynylene comprises two to three carbon atoms (i.e., C₂-C₃ alkynylene). In other embodiments, an alkynylene comprises two carbon atom (i.e., C₂ alkynylene). In other embodiments, an alkynylene comprises five to eight carbon atoms (i.e., C₅-C₈ alkynylene). In other embodiments, an alkynylene comprises three to five carbon atoms (i.e., C₃-C₅ alkynylene).

“Aryl” refers to a radical derived from an aromatic monocyclic or aromatic multicyclic hydrocarbon ring system by removing a hydrogen atom from a ring carbon atom. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl.

The term “carbocycle” refers to a saturated, unsaturated or aromatic rings in which each atom of the ring is carbon. Carbocycle may include 3- to 10-membered monocyclic rings, 6- to 12-membered bicyclic rings, and 6- to 12-membered bridged rings. Each ring of a bicyclic carbocycle may be selected from saturated, unsaturated, and aromatic rings. An aromatic ring, e.g., phenyl, may be fused to a saturated or unsaturated ring, e.g., cyclohexane, cyclopentane, or cyclohexene. Any combination of saturated, unsaturated and aromatic bicyclic rings, as valence permits, are included in the definition of carbocyclic. Exemplary carbocycles include cyclopentyl, cyclohexyl, cyclohexenyl, adamantyl, phenyl, indanyl, and naphthyl. Bicyclic carbocycles may be fused, bridged or spiro-ring systems. In some cases, spiro-ring carbocycles have at least two molecular rings with only one common atom.

The term “halo” or, alternatively, “halogen” or “halide,” means fluoro, chloro, bromo or iodo. In some embodiments, halo is fluoro, chloro, or bromo.

As used herein, the term “haloalkyl” or “haloalkane” refers to an alkyl radical, as defined above, that is substituted by one or more halogen radicals, for example, trifluoromethyl, dichloromethyl, bromomethyl, 2,2,2-trifluoroethyl, 1-fluoromethyl-2-fluoroethyl, and the like. In some embodiments, the alkyl part of the fluoroalkyl radical is optionally further substituted. Examples of halogen substituted alkanes (“haloalkanes”) include halomethane (e.g., chloromethane, bromomethane, fluoromethane, iodomethane), di- and trihalomethane (e.g., trichloromethane, tribromomethane, trifluoromethane, triiodomethane), 1-haloethane, 2-haloethane, 1,2-dihaloethane, 1-halopropane, 2-halopropane, 3-halopropane, 1,2-dihalopropane, 1,3-dihalopropane, 2,3-dihalopropane, 1,2,3-trihalopropane, and any other suitable combinations of alkanes (or substituted alkanes) and halogens (e.g., Cl, Br, F, I, etc.). When an alkyl group is substituted with more than one halogen radicals, each halogen may be independently selected e.g., 1-chloro,2-fluoroethane.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. A bicyclic heterocycle further includes spiro bicylic rings e.g., 5 to 12-membered spiro bicycles.

The term “heterocycloalkyl” refers to a stable 3- to 12-membered non-aromatic ring radical that comprises two to twelve carbon atoms and at least one heteroatom wherein each heteroatom may be selected from N, O, Si, P, B, and S atoms. The heterocycloalkyl may be selected from monocyclic or bicyclic, and fused or bridged ring systems. The heteroatoms in the heterocycloalkyl radical are optionally oxidized. One or more nitrogen atoms, if present, are optionally quaternized. The heterocycloalkyl radical is partially or fully saturated. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, dioxolanyl, thienyl[1,3]dithianyl, decahydroisoquinolyl, imidazolinyl, imidazolidinyl, isothiazolidinyl, isoxazolidinyl, morpholinyl, octahydroindolyl, octahydroisoindolyl, 2-oxopiperazinyl, 2-oxopiperidinyl, 2-oxopyrrolidinyl, oxazolidinyl, piperidinyl, piperazinyl, 4-piperidonyl, pyrrolidinyl, pyrazolidinyl, quinuclidinyl, thiazolidinyl, tetrahydrofuryl, trithianyl, tetrahydropyranyl, thiomorpholinyl, thiamorpholinyl, 1-oxo-thiomorpholinyl, and 1,1-dioxo-thiomorpholinyl.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, acridinyl, benzimidazolyl, benzindolyl, 1,3-benzodioxolyl, benzofuranyl, benzoxazolyl, and thiophenyl (i.e. thienyl).

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, aromatic and non-aromatic substituents of organic compounds. The permissible substituents can be one or more and the same or different for appropriate organic compounds.

In some embodiments, substituents may include any substituents described herein, for example: halogen, hydroxy, oxo (═O), thioxo (═S), cyano (—CN), nitro (—NO₂), imino (═N—H), oximo (═N—OH), hydrazino (═N—NH₂), —R^{bb}—OR^{aa}, —R^{bb}—OC(O)—R^{aa}, —R^{bb}—OC(O)—OR^{aa}, —R^{bb}—OC(O)—N(R^{aa})₂, —R^{bb}—N(R^{aa})₂, —R^{bb}—C(O)R^{aa}, —R^{bb}—C(O)OR^{aa}, —R^{bb}—C(O)N(R^{aa})₂, —R^{bb}—O—R^{cc}—C(O)N(R^{aa})₂, —R^{bb}—N(R^{aa})C(O)OR^{aa}, —R^{bb}—N(R^{aa})C(O)R^{aa}, —R^{bb}—N(R^{aa})S(O)ₜR^{aa} (where t is 1 or 2), —R^{bb}—S(O)ₜR^{aa} (where t is 1 or 2), —R^{bb}—S(O)ₜOR^{aa} (where t is 1 or 2), and —R^{bb}—S(O)ₜN(R^{aa})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{bb}—OR^{aa}, —R^{bb}—OC(O)—R^{aa}, —R^{bb}—OC(O)—OR^{aa}, —R^{bb}—OC(O)—N(R^{aa})₂, —R^{bb}—N(R^{aa})₂, —R^{bb}—C(O)R^{aa}, —R^{bb}—C(O)OR^{aa}, —R^{bb}—C(O)N(R^{aa})₂, —R^{bb}—O—R^{cc}—C(O)N(R^{aa})₂, —R^{bb}—N(R^{aa})C(O)OR^{aa}, —R^{bb}—N(R^{aa})C(O)^{aa}, —R^{bb}—N(R^{aa})S(O)ₜR^{aa} (where t is 1 or 2), —R^{bb}—S(O)ₜR^{aa} (where t is 1 or 2), —R^{bb}—S(O)ₜOR^{aa} (where t is 1 or 2) and —R^{bb}—S(O)ₜN(R^{aa})₂ (where t is 1 or 2); wherein each R^{aa} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{aa}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R^{bb}—OR^{aa}, —R^{bb}—OC(O)—R^{aa}, —R^{bb}—OC(O)—OR^{aa}, —R^{bb}—OC(O)—N(R^{aa})₂, —R^{bb}—N(R^{aa})₂, —R^{bb}—C(O)R^{aa}, —R^{bb}—C(O)OR^{aa}, —R^{bb}—C(O)N(R^{aa})₂, —R^{bb}—O—R^{cc}—C(O)N(R^{aa})₂, —R^{bb}—N(R^{aa})C(O)OR^{aa}, —R^{bb}—N(R^{aa})C(O)R^{aa}, —R^{bb}—N(R^{aa})S(O)ₜR^{aa} (where t is 1 or 2), —R^{bb}—S(O)ₜR^{aa} (where t is 1 or 2), —R^{bb}—S(O)ₜOR^{aa} (where t is 1 or 2) and —R^{bb}—S(O)ₜN(R^{aa})₂ (where t is 1 or 2); and wherein each R^{bb} is independently selected from a direct bond or a straight or branched alkylene, alkenylene, or alkynylene chain, and each R^{cc} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) tale; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; (21) alcohols such as ethanol; (22) ethers such as dimethyl isosorbide or ethoxydiglycol; and (23) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, lactic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

In certain embodiments, the term “cosmetically acceptable salt” means any salt that is cosmetically tolerated if used appropriately for a cosmetic treatment especially if used on or applied to humans and/or mammals. In certain embodiments, these salts include, but are not restricted to the salts used to form base addition salts, either inorganic, such as for example and in a non-limiting sense, lithium, sodium, potassium, calcium, magnesium or aluminum, among others, or organic such as for example and in a non-limiting sense, ethylamine, diethylamine, ethylenediamine, ethanolamine, diethanolamine, arginine, lysine, histidine, or piperazine among others; or acid addition salts, either organic, such as for example and in a non-limiting sense, acetate, citrate, lactate, malonate, maleate, tartrate, fumarate, benzoate, aspartate, glutamate, succinate, oleate, trifluoroacetate, oxalate, pamoate or gluconate among others, or inorganic, such as for example and in a non-limiting sense, chloride, sulfate, borate, or carbonate among others.

A “cosmetically effective amount” as used herein refers to the amount of a compound sufficient to improve the outward physical appearance of a subject. It is to be understood that a “cosmetically effective” amount can vary from subject to subject, due to numerous factors including for example age, weight, general condition of the subject, the condition being treated, and the severity of the condition being treated.

The phrase “cosmetically acceptable excipient” or “cosmetically acceptable carrier” as used herein comprises a cream base, an oil-in-water emulsion, a water-in-oil emulsion, a gel, or the like. The skilled artisan will understand that the appropriate carriers typically will contain ingredients, such as those typically found in the cosmetic and cosmeceutical fields: oils, waxes or other standard fatty substances, or conventional gelling agents and/or thickeners; emulsifiers; moisturizing agents; emollients; sunscreens; hydrophilic or lipophilic active agents; agents for combatting free radicals; preservatives; basifying or acidifying agents; fragrances; surfactants; fillers; natural products or extracts of natural product, such as aloe or green tea extract; vitamins; or coloring materials.

As used herein, the term “cosmetically acceptable carrier, diluent, or excipient” refers to a substance that can be safely used in contact with tissues (such as the skin, hair, nails, etc.) and imparts the desired cosmetic effect, without causing any undesirable side effects such as irritation, allergic responses, or toxicity. Such a substance can act as a medium to deliver the active ingredient(s) to the target site, improve the cosmetic formulation's texture or appearance, or otherwise enhance the product's overall quality or stability. Cosmetically acceptable carriers, diluents, or excipients should be suitable for topical application and can include, but are not limited to, water, oils, alcohols, silicones, emulsifiers, preservatives, colorants, fragrances, surfactants, thickeners, and the like. The exact choice of a cosmetically acceptable carrier, diluent or excipient will depend on the particular cosmetic formulation, the specific active ingredient(s), and the intended use of the product.

The term “in vivo” generally refers to an event that takes place in a subject's body.

The term “in vitro” generally refers to an event that takes place outside of a subject's body. For example, an in vitro assay encompasses any assay run outside of a subject. In vitro assays encompass cell-based assays in which cells alive or dead are employed. In vitro assays also encompass a cell-free assay in which no intact cells are employed.

“Optional” or “optionally” means that the subsequently described event of circumstances may or may not occur, and that the description includes instances where the event or circumstance occurs and instances in which it does not. For example, “optionally substituted aryl” means that the aryl group may or may not be substituted and that the description includes both substituted aryl groups and aryl groups having no substitution.

“Pharmaceutically acceptable carrier, diluent or excipient” includes without limitation any adjuvant, carrier, excipient, glidant, sweetening agent, diluent, preservative, dye, colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In certain embodiments, the term “prevent” or “preventing” as related to a disease or disorder may refer to a compound that, in a statistical sample, reduces the occurrence of the disorder or condition in the treated sample relative to an untreated control sample, or delays the onset or reduces the severity of one or more symptoms of the disorder or condition relative to the untreated control sample.

The terms “treat,” “treating” or “treatment,” as used herein, may include alleviating, abating or ameliorating a condition or disease symptoms, preventing additional symptoms, ameliorating or preventing the underlying causes of symptoms, inhibiting the condition or disease, e.g., arresting the development of the condition or disease, relieving the condition or disease, causing regression of the condition or disease, relieving a condition caused by the condition or disease, or stopping the symptoms of the condition or disease either prophylactically and/or therapeutically.

### Compounds

The present disclosure provides compounds, salts thereof, and compositions and formulations thereof, for treating, preventing, or providing a relief for a disease, disorder, or condition in a subject in need thereof. In some embodiments, the compounds, salts thereof, and compositions and formulations thereof, are useful for treating or providing a relief for a disease, disorder, or condition in a subject in need thereof. The compounds comprise a structural formula (I), (II), (III), (IV), or (V), or a salt thereof. The compounds are selected from those forth in Tables 1-6, or salts thereof, or any subset thereof. The compounds and salts thereof disclosed herein may be used in method(s) of the disclosure. The following is a discussion of compounds and salts thereof that may be used in the methods of the disclosure.

In certain aspects, disclosed herein is a compound having a structure of Formula (I):

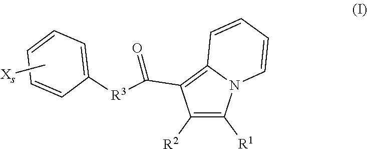

or a salt thereof,
wherein
R¹ is selected from optionally substituted C₁₋₆ alkyl, C₂₋₆ alkenyl, and C₂₋₆ alkynyl, —OR^{1a}, —SR^{1a}, —C(O)R^{1a}, —C(O)OR^{1a}, —OC(O)R^{1a}, —OC(O)N(R^{1a})₂, —NR^{1a}S(O)₂R^{1a}, —C(O)N(R^{1a})₂, —S(O)₂(R^{1a}), —S(O)₂N(R^{1a})₂, optionally substituted C₃-C₁₂ carbocycle, and optionally substituted 5- to 14-membered heterocycle;
   each R^{1a} is independently selected at each occurrence from C₁₋₆ alkyl, C₅-C₈ carbocycle, and C₅-C₈ heteroaryl, each of which is optionally substituted with one or more substituents selected from C₁₋₆ alkyl, halogen, —NO₂, —NR^{1b}₂, phenyl, —SR^{1b}, —OR^{1b}, and 3- to 12-membered heterocycle, wherein R^{1b} is hydrogen or C₁-C₆ alkyl;
R² is selected from hydrogen, —OH, halogen, C₁₋₆ alkoxy, C₁₋₆ haloalkoxy, —NH₂, and C₁₋₆ alkylamino;
R³ is selected from optionally substituted C₁₋₆ alkylene, optionally substituted C₁₋₆ haloalkyl, —CO—, —NR^{1b}—, and —O—;
each X is independently selected at each occurrence from —OR^{1b}, halogen, C₁₋₆ alkyl, or C₁₋₆ haloalkyl; and
s is selected from 0, 1, 2, and 3.

In some embodiments, R¹ is selected from optionally substituted C₁₋₆ alkyl, C₂₋₆ alkenyl, and C₂₋₆ alkynyl, —OR^{1a}, —SR^{1a}, —C(O)R^{1a}, and —C(O)OR^{1a}. In some embodiments, R¹ is optionally substituted C₁₋₆ alkyl. In some embodiments, R¹ is optionally substituted C₂₋₆ alkenyl. In some embodiments, R¹ is optionally substituted C₂₋₆ alkynyl. In some embodiments, R¹ is —SR^{1a}. In some embodiments, R¹ is —C(O)R^{1a}. In some embodiments, R¹ is —OC(O)R^{1a}. In some embodiments, R¹ is —OR^{1a}.

In some embodiments, R^{1a} is optionally substituted C₅-C₈ carbocycle. In some embodiments, R^{1a} is C₅-C₈ carbocycle optionally substituted with one or more substituents. In some embodiments, the one or more substituents are selected at each occurrence from C₁₋₆ alkyl, halogen, and —NO₂. In some embodiments, the one or more substituents are selected at each occurrence from C₁₋₆ alkyl. In some embodiments, the one or more substituents are selected at each occurrence from halogen. In some embodiments, the one or more substituents are and —NO₂. In some embodiments, R^{1a} is optionally substituted C₅ carbocycle or optionally substituted C₆ carbocycle. In some embodiments, R^{1a} is optionally substituted C₅ carbocycle. In some embodiments, R^{1a} is

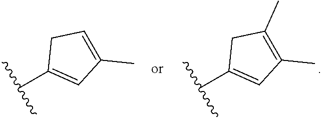

In some embodiments, R^{1a} is

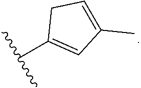 [SMILES: *C1=CC(C)=CC1]

In some embodiments, R^{1a} is

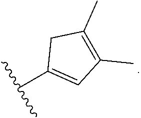 [SMILES: *C1=CC(C)=C(C)C1]

In some embodiments, R^{1a} is optionally substituted C₆ carbocycle. In some embodiments, R^{1a} is selected from

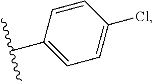 [SMILES: *c1ccc(Cl)cc1]

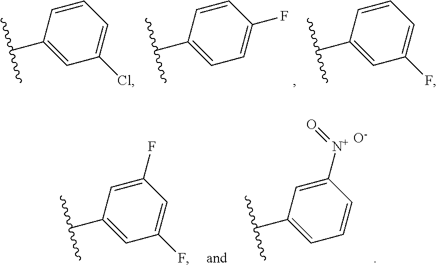

In some embodiments, R^{1a} is

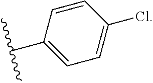 [SMILES: *c1ccc(Cl)cc1]

In some embodiments, R^{1a} is

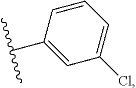 [SMILES: *c1cccc(Cl)c1]

In some embodiments, R^{1a} is

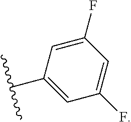 [SMILES: *c1cc(F)cc(F)c1]

In some embodiments, R^{1a} is

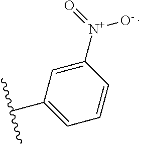 [SMILES: *c1cccc([N+](=O)[O-])c1]

In some embodiments, R^{1a} is

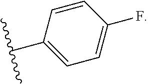 [SMILES: *c1ccc(F)cc1]

In some embodiments, R^{1a} is

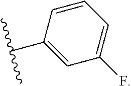 [SMILES: *c1cccc(F)c1]

In some embodiments, R² is hydrogen. In some embodiments, R² is halogen. In some embodiments, R² is —Cl. In some embodiments, R² is —F. In some embodiments, R² is —NH₂.

In some embodiments, R³ is selected from optionally substituted C₁₋₆ alkylene. In some embodiments, R³ is C₁ alkylene. In some embodiments, R³ is —CH₂—. In some embodiments, R³ is —NR^{1b}—. In some embodiments, R^{1b} is hydrogen. In some embodiments, R^{1b} is selected from C₁-C₆ alkyl. In some embodiments, R³ is —NH—.

In some embodiments, X is —OR^{1b}. In some embodiments, R^{1b} is hydrogen. In some embodiments, R^{1b} is selected from C₁-C₆ alkyl. In some embodiments, X is —OH. In some embodiments, X is —OCH₃. In some embodiments, X is halogen. In some embodiments, X is —Cl. In some embodiments, X is —F.

In some embodiments, s is 1 or 2. In some embodiments, s is 1. In some embodiments, s is 2.

In some embodiments, a compound having a structural Formula (I) is selected from those set forth in Table 1, and a salt thereof.

TABLE 1
Example Compounds of Formula (I)

| ID # | Chemical Structure | Chemical Name |
|---|---|---|
| I-1 | 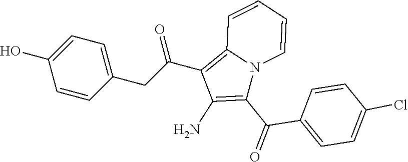 [SMILES: Nc1c(C(=O)Cc2ccc(O)cc2)c2ccccn2c1C(=O)c1ccc(Cl)cc1] | 1-(2-amino-3-(4- chlorobenzoyl)indolizin- 1-yl)-2-(4- hydroxyphenyl)ethan-1- one |
| I-2 | 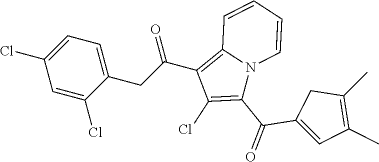 [SMILES: CC1=C(C)CC(C(=O)c2c(Cl)c(C(=O)Cc3ccc(Cl)cc3Cl)c3ccccn23)=C1] | 1-(2-chloro-3-(3,4- dimethylcyclopenta-1,3- diene-1- carbonyl)indolizin-1-yl)- 2-(2,4- dichlorophenyl)ethan-1- one |
| I-3 | 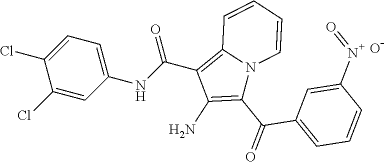 | 2-amino-N-(3,4- dichlorophenyl)-3-(3- nitrobenzoyl)indolizine-1- carboxamide |
| I-4 | 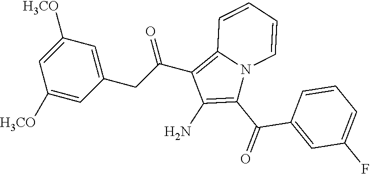 [SMILES: COc1cc(CC(=O)c2c(N)c(C(=O)c3cccc(F)c3)n3ccccc23)cc(OC)c1] | 1-(2-amino-3-(3- fluorobenzoyl)indolizin-1- yl)-2-(3,5- dimethoxyphenyl)ethan-1- one |
| I-5 | 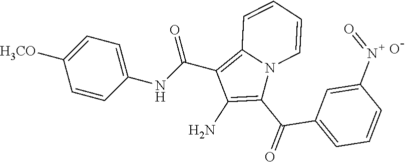 | 2-amino-N-(4- methoxyphenyl)-3-(3- nitrobenzoyl)indolizine-1- carboxamide |
| I-6 | 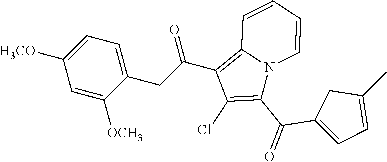 [SMILES: COc1ccc(CC(=O)c2c(Cl)c(C(=O)C3=CC=C(C)C3)n3ccccc23)c(OC)c1] | 1-(2-chloro-3-(4- methylcyclopenta-1,3- diene-1- carbonyl)indolizin-1-yl)- 2-(2,4- dimethoxyphenyl)ethan-1- one |
| I-7 | 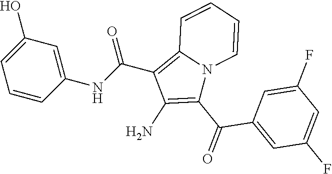 [SMILES: Nc1c(C(=O)Nc2cccc(O)c2)c2ccccn2c1C(=O)c1cc(F)cc(F)c1] | 2-amino-3-(3,5- difluorobenzoyl)-N-(3- hydroxyphenyl)indolizine- 1-carboxamide |
| I-8 | 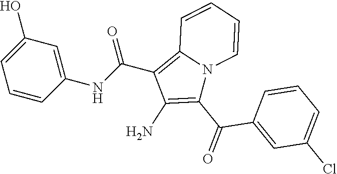 [SMILES: Nc1c(C(=O)Nc2cccc(O)c2)c2ccccn2c1C(=O)c1cccc(Cl)c1] | 2-amino-3-(3- chlorobenzoyl)-N-(3- hydroxyphenyl)indolizine- 1-carboxamide |

In some embodiments, the compound having a structural Formula (I) is

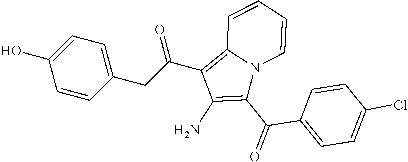 [SMILES: Nc1c(C(=O)Cc2ccc(O)cc2)c2ccccn2c1C(=O)c1ccc(Cl)cc1]

or a salt thereof. In some embodiments, the compound having structural Formula (I) is

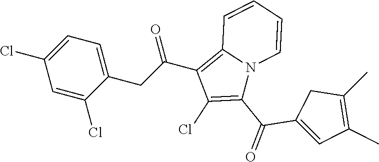 [SMILES: CC1=C(C)CC(C(=O)c2c(Cl)c(C(=O)Cc3ccc(Cl)cc3Cl)c3ccccn23)=C1]

or a salt thereof. In some embodiments, the compound having a structural Formula (I) is

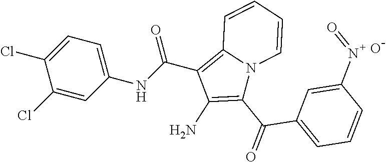

or a salt thereof. In some embodiments, the compound having a structural Formula (I) is

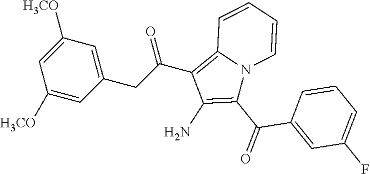 [SMILES: COc1cc(CC(=O)c2c(N)c(C(=O)c3cccc(F)c3)n3ccccc23)cc(OC)c1]

or a salt thereof. In some embodiments, the compound having a structural Formula (I) is

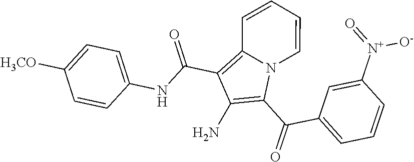 [SMILES: COc1ccc(NC(=O)c2c(N)c(C(=O)c3cccc([N+](=O)[O-])c3)n3ccccc23)cc1]

or a salt thereof. In some embodiments, the compound having a structural Formula (I) is

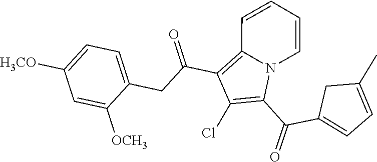 [SMILES: COc1ccc(CC(=O)c2c(Cl)c(C(=O)C3=CC=C(C)C3)n3ccccc23)c(OC)c1]

or a salt thereof. In some embodiments, the compound having a structural Formula (I) is

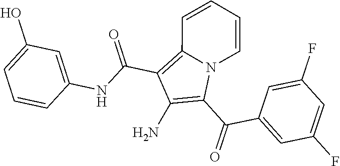 [SMILES: Nc1c(C(=O)Nc2cccc(O)c2)c2ccccn2c1C(=O)c1cc(F)cc(F)c1]

or a salt thereof. In some embodiments, the compound having a structural Formula (I) is

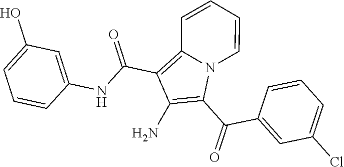 [SMILES: Nc1c(C(=O)Nc2cccc(O)c2)c2ccccn2c1C(=O)c1cccc(Cl)c1]

or a salt thereof.

In certain aspects, disclosed herein is a compound represented by Formula (II):

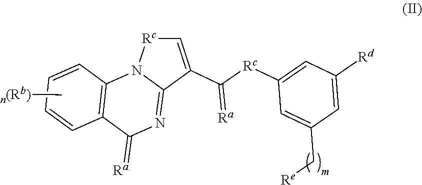

or a salt thereof,

wherein
each R^{a} is independently selected from O, S, and —NH;
each R^{b} is independently selected at each occurrence from hydrogen, —OR^{b1}, halogen, C₁₋₆ alkoxy, C₁₋₆ haloalkoxy, —NR^{b1}₂, and C₁₋₆ alkylamino;
each R^{c} is independently selected from O, S, or NH;
R^{d} is selected from hydrogen, —OR^{b1}, halogen, C₁₋₆ alkoxy, C₁₋₆ haloalkoxy, —NR^{b1}₂, and C₁₋₆ alkylamino;
R^{e} is selected from —C₁-C₆ alkyl, optionally substituted C₅-C₁₀ carbocycle, and optionally substituted C₅-C₁₀ heterocycle;
n is selected from 0, 1, 2, or 3; and
m is selected from 1, 2, or 3,
wherein R^{b1} is hydrogen or C₁-C₆ alkyl.

In some embodiments, R^{a} is O. In some embodiments, R^{a} is S. In some embodiments, R^{a} is NH.

In some embodiments, R^{b} is hydrogen. In some embodiments, R^{b} is —OR^{b1}. In some embodiments, R^{b1} is hydrogen. In some embodiments, R^{b1} is selected from C₁-C₆ alkyl. In some embodiments, R^{b} is —OH. In some embodiments, R^{b} is halogen. In some embodiments, R^{b} is —Cl. In some embodiments, R^{b} is —F.

In some embodiments, n is 0, 1, or 2. In some embodiments, n is 0. In some embodiments, n is 1. In some embodiments, n is 2.

In some embodiments, R^{c} is O. In some embodiments, R^{c} is S. In some embodiments, R^{c} is NH.

In some embodiments, R^{d} is selected from hydrogen, —OR^{b1}, and halogen. In some embodiments, R^{d} is hydrogen. In some embodiments, R^{d} is —OR^{b1}. In some embodiments, R^{b1} is hydrogen. In some embodiments, R^{b1} is selected from C₁-C₆ alkyl. In some embodiments, R^{d} is —OH. In some embodiments, R^{d} is halogen. In some embodiments, R^{d} is —Cl. In some embodiments, R^{d} is —F.

In some embodiments, R^{e} is optionally substituted C₅-C₁₀ carbocycle. In some embodiments, R^{e} is optionally substituted C₅-C₁₀ heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, the heterocycle has three nitrogen atoms. In some embodiments, R^{e} is selected from the group consisting of:

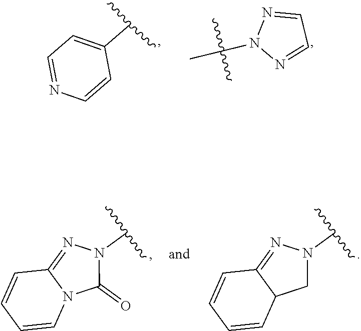 [SMILES: *c1ccncc1 ; *n1nccn1 ; *n1nc2ccccn2c1=O ; *N1CC2C=CC=CC2=N1]

In some embodiments, R^{e} is

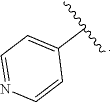 [SMILES: *c1ccncc1]

In some embodiments, R^{e} is

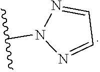 [SMILES: *n1nccn1]

In some embodiments, R^{e} is

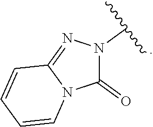 [SMILES: *n1nc2ccccn2c1=O]

In some embodiments, R^{e} is

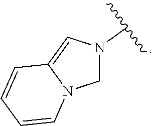 [SMILES: *N1C=C2C=CC=CN2C1]

In some embodiments, m is 1 or 2. In some embodiments, m is 1. In some embodiments, m is 2.

In some embodiments, a compound having a structural Formula (II) is selected from those set forth in Table 2, and a salt thereof.

TABLE 2
Example Compounds of Formula (II)

| ID # | Chemical Structure | Chemical Name |
|---|---|---|
| II-1 | 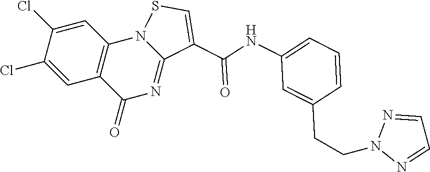 [SMILES: O=C(Nc1cccc(CCn2nccn2)c1)c1csn2c1nc(=O)c1cc(Cl)c(Cl)cc12] | N-(3-(2-(2H-1,2,3-triazol-2- yl)ethyl)phenyl)-7,8-dichloro- 5-oxo-5H-isothiazolo[2,3- a]quinazoline-3-carboxamide |
| II-2 | 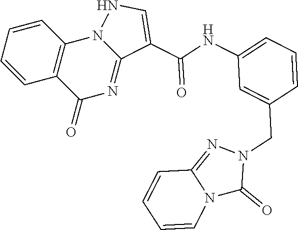 [SMILES: O=C(Nc1cccc(Cn2nc3ccccn3c2=O)c1)c1c[nH]n2c1nc(=O)c1ccccc12] | 5-oxo-N-(3-((3-oxo- [1,2,4]triazolo[4,3-a]pyridin- 2(3H)-yl)methyl)phenyl)-1,5- dihydropyrazolo[1,5- a]quinazoline-3-carboxamide |
| II-3 | 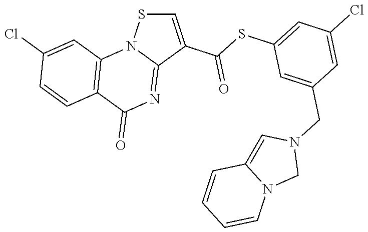 [SMILES: O=C(Sc1cc(Cl)cc(CN2C=C3C=CC=CN3C2)c1)c1csn2c1nc(=O)c1ccc(Cl)cc12] | S-(3-chloro-5-(imidazo[1,5- a]pyridin-2(3H)- ylmethyl)phenyl)8-chloro-5- oxo-5H-isothiazolo[2,3- a]quinazoline-3-carbothioate |
| II-4 | 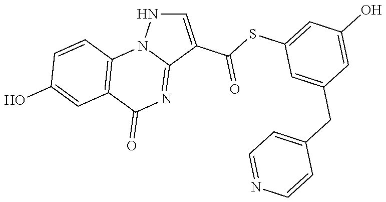 [SMILES: O=C(Sc1cc(O)cc(Cc2ccncc2)c1)c1c[nH]n2c1nc(=O)c1cc(O)ccc12] | S-(3-hydroxy-5-(pyridin-4- ylmethyl)phenyl)7-hydroxy-5- oxo-1,5-dihydropyrazolo[1,5- a]quinazoline-3-carbothioate |

In some embodiments, the compound having a structural Formula (II) is

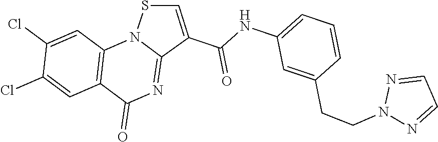 [SMILES: O=C(Nc1cccc(CCn2nccn2)c1)c1csn2c1nc(=O)c1cc(Cl)c(Cl)cc12]

or a salt thereof. In some embodiments, the compound having a structural Formula (II) is

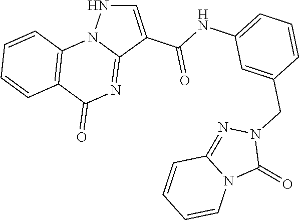 [SMILES: O=C(Nc1cccc(Cn2nc3ccccn3c2=O)c1)c1c[nH]n2c1nc(=O)c1ccccc12]

or a salt thereof. In some embodiments, the compound having a structural Formula (II) is

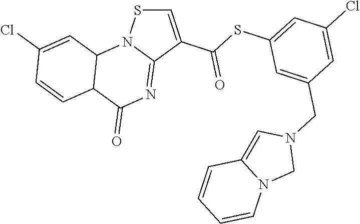 [SMILES: O=C(Sc1cc(Cl)cc(CN2C=C3C=CC=CN3C2)c1)C1=CSN2C1=NC(=O)C1C=CC(Cl)=CC12]

or a salt thereof. In some embodiments, the compound having a structural Formula (II) is

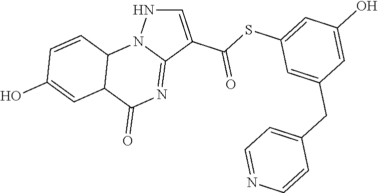 [SMILES: O=C(Sc1cc(O)cc(Cc2ccncc2)c1)C1=CNN2C1=NC(=O)C1C=C(O)C=CC12]

or a salt thereof.

In certain aspects, disclosed herein is a compound represented by Formula (III):

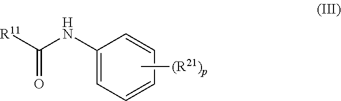

or a salt thereof,
R¹¹ is selected from —CH₂R¹², —OR¹², —SR¹², —C(O)R¹², —C(O)OR¹², —OC(O)R¹², —OC(O)N(R¹²)₂, —NR¹²S(O)₂R¹², —C(O)N(R¹²)₂, —S(O)₂(R¹²), —S(O)₂N(R¹²)₂, optionally substituted C₃-C₁₂ carbocycle, and optionally substituted 5- to 14-membered heterocycle;
   each R¹² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle;
each R²¹ is independently selected at each occurrence from —OH, halogen, C₁₋₆ alkyl, C₁₋₆ haloalkyl, OR²², —SR²², —C(O)R²², —C(O)OR²², —OC(O)R²², —OCH₂C(O)R²², —OC(O)N(R²²)₂, —OCH₂C(O)NHR²², —OCH₂C(O)N(R²²)₂, —NR²²S(O)₂R²², —C(O)N(R²²)₂, —S(O)₂(R²²), —S(O)₂NHR²², —S(O)₂N(R²²)₂, —NHC(O)R²², optionally substituted C₃-C₁₂ carbocycle, and optionally substituted 5- to 14-membered heterocycle;
   each R²² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle; and p is selected from 0, 1, 2, 3, or 4.

In some embodiments, the compound has a structure of Formula (III-1):

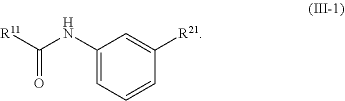

In some embodiments, the compound has a structure of Formula (III-2):

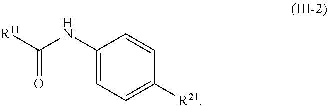

In some embodiments, the compound has a structure of Formula (III-3):

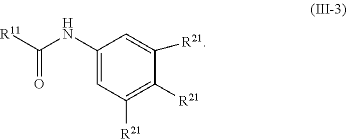

In some embodiments, R¹¹ is —CH₂R¹². In some embodiments, R¹² is optionally substituted C₃-C₁₂ carbocycle or optionally substituted 5- to 14-membered heterocycle. In some embodiments, R¹² is selected from optionally substituted C₃-C₁₂ carbocycle. In some embodiments, R¹² is selected from optionally substituted C₃ carbocycle. In some embodiments, R¹² is selected from optionally substituted C₃ carbocycle. In some embodiments, R¹² is selected from optionally substituted C₄ carbocycle. In some embodiments, R¹² is selected from optionally substituted C₅ carbocycle. In some embodiments, R¹² is selected from optionally substituted C₆ carbocycle. In some embodiments, R¹² is selected from optionally substituted C₇ carbocycle. In some embodiments, R¹² is selected from optionally substituted C₈ carbocycle. In some embodiments, R¹² is selected from optionally substituted C₉ carbocycle. In some embodiments, R¹² is selected from optionally substituted C₁₀ carbocycle. In some embodiments, R¹² is selected from optionally substituted C₁₁ carbocycle. In some embodiments, R¹² is selected from optionally substituted C₁₂ carbocycle. In some embodiments, R¹² is selected from optionally substituted 5- to 14-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, the heterocycle has three nitrogen atoms. In some embodiments, R¹² is selected from optionally substituted 5-membered heterocycle. In some embodiments, R¹² is selected from optionally substituted 6-membered heterocycle. In some embodiments, R¹² is selected from optionally substituted 7-membered heterocycle. In some embodiments, R¹² is selected from optionally substituted 8-membered heterocycle. In some embodiments, R¹² is selected from optionally substituted 9-membered heterocycle. In some embodiments, R¹² is selected from optionally substituted 10-membered heterocycle. In some embodiments, R¹² is selected from optionally substituted 11-membered heterocycle. In some embodiments, R¹² is selected from optionally substituted 12-membered heterocycle. In some embodiments, R¹² is selected from optionally substituted 13-membered heterocycle. In some embodiments, R¹² is selected from optionally substituted 14-membered heterocycle. In some embodiments, R¹² is selected from

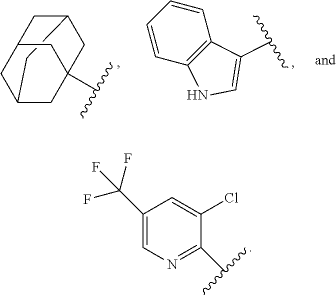 [SMILES: *C12CC3CC(CC(C3)C1)C2 ; *c1c[nH]c2ccccc12 ; *c1ncc(C(F)(F)F)cc1Cl]

In some embodiments, R¹² is

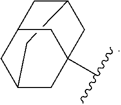 [SMILES: *C12CC3CC(CC(C3)C1)C2]

In some embodiments, R¹² is

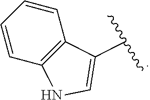 [SMILES: *c1c[nH]c2ccccc12]

In some embodiments, R¹² is

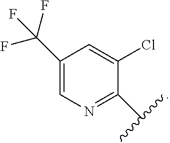 [SMILES: *c1ncc(C(F)(F)F)cc1Cl]

In some embodiments, R¹¹ is selected from

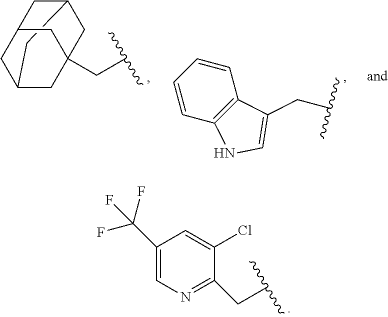 [SMILES: *CC12CC3CC(CC(C3)C1)C2 ; *Cc1c[nH]c2ccccc12 ; *Cc1ncc(C(F)(F)F)cc1Cl]

In some embodiments, R¹¹ is

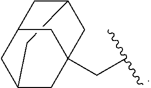 [SMILES: *CC12CC3CC(CC(C3)C1)C2]

In some embodiments, R¹¹ is

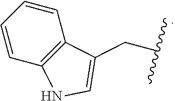 [SMILES: *Cc1c[nH]c2ccccc12]

In some embodiments, R¹¹ is

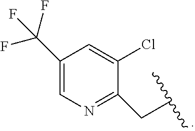 [SMILES: *Cc1ncc(C(F)(F)F)cc1Cl]

In some embodiments, R¹¹ is —OR¹². In some embodiments, R¹¹ is —OH. In some embodiments, R¹¹ is —SR¹². In some embodiments, R¹¹ is optionally substituted C₃-C₁₂ carbocycle or optionally substituted 5- to 14-membered heterocycle. In some embodiments, R¹¹ is selected from optionally substituted C₃-C₁₂ carbocycle. In some embodiments, R¹¹ is selected from optionally substituted C₃ carbocycle. In some embodiments, R¹¹ is selected from optionally substituted C₄ carbocycle. In some embodiments, R¹¹ is selected from optionally substituted C₅ carbocycle. In some embodiments, R¹¹ is selected from optionally substituted C₆ carbocycle. In some embodiments, R¹¹ is selected from optionally substituted C₇ carbocycle. In some embodiments, R¹¹ is selected from optionally substituted C₈ carbocycle. In some embodiments, R¹¹ is selected from optionally substituted C₉ carbocycle. In some embodiments, R¹¹ is selected from optionally substituted C₁₀ carbocycle. In some embodiments, R¹¹ is selected from optionally substituted C₁ carbocycle. In some embodiments, R¹¹ is selected from optionally substituted C₁₂ carbocycle. In some embodiments, R¹¹ is selected from optionally substituted 5- to 14-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has at least one sulfur atom. In some embodiments, the heterocycle has a sulfur atom. In some embodiments, R¹¹ is optionally substituted 5-membered heterocycle. In some embodiments, R¹¹ is optionally substituted 6-membered heterocycle. In some embodiments, R¹¹ is optionally substituted 7-membered heterocycle. In some embodiments, R¹¹ is optionally substituted 8-membered heterocycle. In some embodiments, R¹¹ is optionally substituted 9-membered heterocycle. In some embodiments, R¹¹ is optionally substituted 10-membered heterocycle. In some embodiments, R¹¹ is optionally substituted 11-membered heterocycle. In some embodiments, R¹¹ is optionally substituted 12-membered heterocycle. In some embodiments, R¹¹ is optionally substituted 13-membered heterocycle. In some embodiments, R¹¹ is optionally substituted 14-membered heterocycle. In some embodiments, R¹¹ is

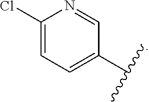 [SMILES: *c1ccc(Cl)nc1]

In some embodiments, R¹¹ is

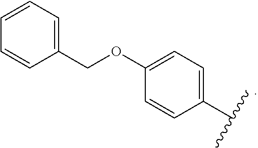 [SMILES: *c1ccc(OCc2ccccc2)cc1]

In some embodiments, R¹¹ is

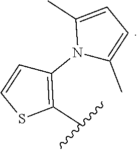 [SMILES: *c1sccc1-n1c(C)ccc1C]

In some embodiments, R¹¹ is

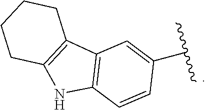 [SMILES: *c1ccc2[nH]c3c(c2c1)CCCC3]

In some embodiments, R²¹ is —OH. In some embodiments, R²¹ is C₁₋₆ alkyl. In some embodiments, R²¹ is C₁ alkyl. In some embodiments, R²¹ is C₂ alkyl. In some embodiments, R²¹ is C₃ alkyl. In some embodiments, R²¹ is C₄ alkyl. In some embodiments, R²¹ is C₅ alkyl. In some embodiments, R²¹ is C₆ alkyl.

In some embodiments, R²¹ is —OCH₂C(O)NHR²². In some embodiments, R²² is C₁ alkyl. In some embodiments, R²² is C₂alkyl. In some embodiments, R²² is C₃ alkyl. In some embodiments, R²² is C₄ alkyl. In some embodiments, R²¹ is —OCH₂C(O)NHCH₃. In some embodiments, R²¹ is —OCH₂C(O)NHCH₂CH₃. In some embodiments, R²¹ is —OCH₂C(O)NHCH₂CH₂CH₃.

In some embodiments, R²¹ is —S(O)₂NHR²². In some embodiments, R²² is optionally substituted 5- to 14-membered heterocycle. In some embodiments, R²² is optionally substituted 5-membered heterocycle. In some embodiments, R²² is optionally substituted 6-membered heterocycle. In some embodiments, R²² is optionally substituted 7-membered heterocycle. In some embodiments, R²² is optionally substituted 8-membered heterocycle. In some embodiments, R²² is optionally substituted 9-membered heterocycle. In some embodiments, R²² is optionally substituted 10-membered heterocycle. In some embodiments, R²² is optionally substituted 11-membered heterocycle. In some embodiments, R²² is optionally substituted 12-membered heterocycle. In some embodiments, R²² is optionally substituted 13-membered heterocycle. In some embodiments, R²² is optionally substituted 14-membered heterocycle. In some embodiments, R²² is

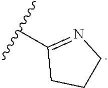 [SMILES: *C1=NCCC1]

In some embodiments, R²¹ is

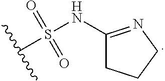 [SMILES: *S(=O)(=O)NC1=NCCC1]

In some embodiments, R²¹ is —NHC(O)R²². In some embodiments, R²² is selected from optionally substituted C₃-C₁₂ carbocycle. In some embodiments, R²² is optionally substituted C₃ carbocycle. In some embodiments, R²² is optionally substituted C₄ carbocycle. In some embodiments, R²² is optionally substituted C₅ carbocycle. In some embodiments, R²² is optionally substituted C₆ carbocycle. In some embodiments, R²² is optionally substituted C₇ carbocycle. In some embodiments, R²² is optionally substituted C₈ carbocycle. In some embodiments, R²² is optionally substituted C₉ carbocycle. In some embodiments, R²² is optionally substituted C₁₀ carbocycle. In some embodiments, R²² is optionally substituted C₁₁ carbocycle. In some embodiments, R²² is optionally substituted C₁₂ carbocycle. In some embodiments, R²¹ is optionally substituted 5- to 14-membered heterocycle. In some embodiments, R²¹ is optionally substituted 5-membered heterocycle. In some embodiments, R²¹ is optionally substituted 6-membered heterocycle. In some embodiments, R²¹ is optionally substituted 7-membered heterocycle. In some embodiments, R²¹ is optionally substituted 8-membered heterocycle. In some embodiments, R²¹ is optionally substituted 9-membered heterocycle. In some embodiments, R²¹ is optionally substituted 10-membered heterocycle. In some embodiments, R²¹ is optionally substituted 11-membered heterocycle. In some embodiments, R²¹ is optionally substituted 12-membered heterocycle. In some embodiments, R²¹ is optionally substituted 13-membered heterocycle. In some embodiments, R²¹ is optionally substituted 14-membered heterocycle. In some embodiments, R²¹ is

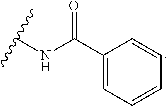 [SMILES: *NC(=O)c1ccccc1]

In some embodiments, R²¹ is optionally substituted 5- to 14-membered heterocycle. In some embodiments, R²¹ is selected at each occurrence from

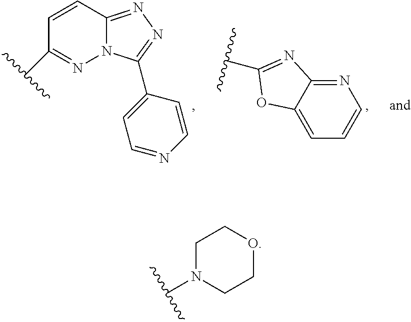 [SMILES: *c1ccc2nnc(-c3ccncc3)n2n1 ; *c1nc2ncccc2o1 ; *N1CCOCC1]

In some embodiments, R²¹ is selected from —OH, —CH₃,

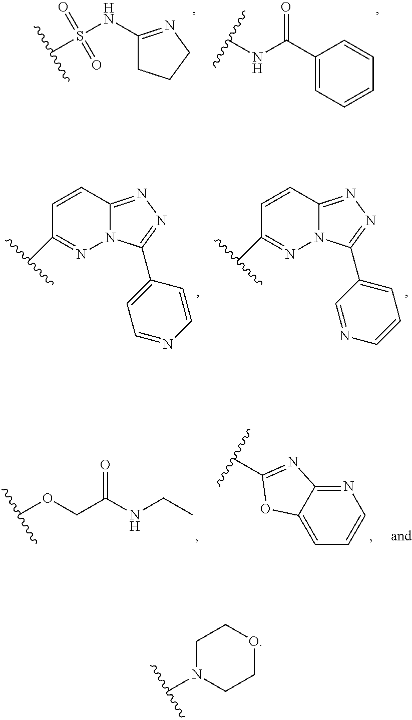

In some embodiments, R²¹ is —OH. In some embodiments, R²¹ is —CH₃. In some embodiments, R²¹ is

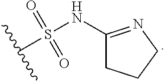 [SMILES: *S(=O)(=O)NC1=NCCC1]

In some embodiments, R²¹ is

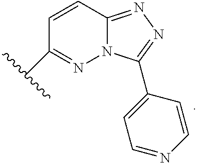 [SMILES: *c1ccc2nnc(-c3ccncc3)n2n1]

In some embodiments, R²¹ is

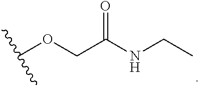 [SMILES: *OCC(=O)NCC]

In some embodiments, R²¹ is

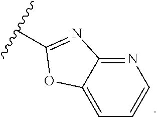 [SMILES: *c1nc2ncccc2o1]

In some embodiments, R²¹ is

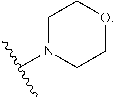 [SMILES: *N1CCOCC1]

In some embodiments, R²¹ is

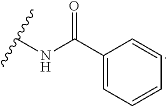 [SMILES: *NC(=O)c1ccccc1]

In some embodiments, R²¹ is

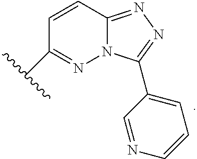 [SMILES: *c1ccc2nnc(-c3cccnc3)n2n1]

In some embodiments, p is selected from 1, 2, and 3. In some embodiments, p is 1. In some embodiments, p is 2. In some embodiments, p is 3.

In some embodiments, a compound having a structural Formula (III) is selected from those set forth in Table 3, and a salt thereof.

TABLE 3
Example Compounds of Formula (III)

| ID # | Chemical Structure | Chemical Name |
|---|---|---|
| III-1 | 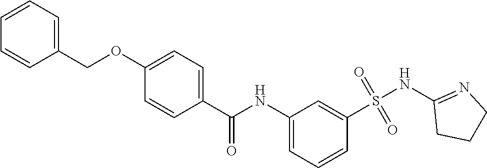 [SMILES: O=C(Nc1cccc(S(=O)(=O)NC2=NCCC2)c1)c1ccc(OCc2ccccc2)cc1] | 4-(benzyloxy)-N-{3-[(3,4- dihydro-2H-pyrrol-5- yl)sulfamoyl]phenyl}benzamide |
| III-2 | 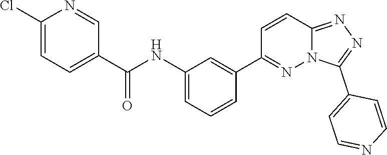 [SMILES: O=C(Nc1cccc(-c2ccc3nnc(-c4ccncc4)n3n2)c1)c1ccc(Cl)nc1] | 6-chloro-N-{3-[3-(pyridin-4-yl)- [1,2,4]triazolo[4,3-b]pyridazin- 6-yl]phenyl}pyridine-3- carboxamide |
| III-3 | 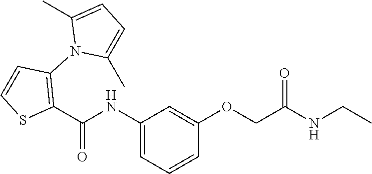 [SMILES: CCNC(=O)COc1cccc(NC(=O)c2sccc2-n2c(C)ccc2C)c1] | 3-(2,5-dimethyl-1H-pyrrol-1- yl)-N-{3- [(ethylcarbamoyl)methoxy]phenyl} thiophene- 2-carboxamide |
| III-4 | 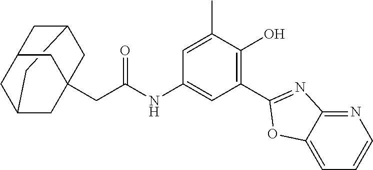 [SMILES: Cc1cc(NC(=O)CC23CC4CC(CC(C4)C2)C3)cc(-c2nc3ncccc3o2)c1O] | 2-(1-adamantyl)-N-(4-hydroxy- 3-methy1-5-[1,3]oxazolo[4,5- b]pyridin-2-ylphenyl)acetamide |
| III-5 | 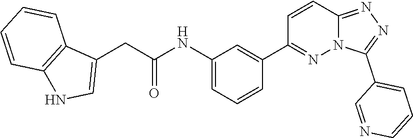 [SMILES: O=C(Cc1c[nH]c2ccccc12)Nc1cccc(-c2ccc3nnc(-c4cccnc4)n3n2)c1] | 2-(1H-indol-3-yl)-N-{3-[3- (pyridin-3-yl)- [1,2,4]triazolo[4,3-b]pyridazin- 6-yl]phenyl}acetamide |
| III-6 | 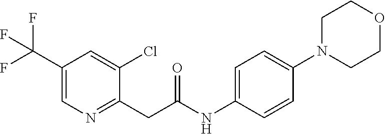 [SMILES: O=C(Cc1ncc(C(F)(F)F)cc1Cl)Nc1ccc(N2CCOCC2)cc1] | 2-[3-chloro-5- (trifluoromethyl)pyridin-2-yl]- N-[4-(morpholin-4- yl)phenyl]acetamide |
| III-7 | 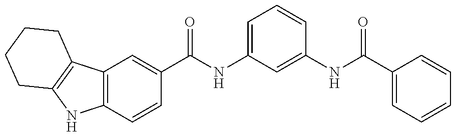 [SMILES: O=C(Nc1cccc(NC(=O)c2ccc3[nH]c4c(c3c2)CCCC4)c1)c1ccccc1] | N-(3-benzamidophenyl)-2,3,4,9- tetrahydro- 1H-carbazole-6- carboxamide |

In some embodiments, the compound having a structural Formula (III) is

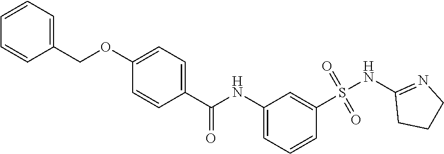 [SMILES: O=C(Nc1cccc(S(=O)(=O)NC2=NCCC2)c1)c1ccc(OCc2ccccc2)cc1]

or a salt thereof. In some embodiments, the compound having a structural Formula (III) is

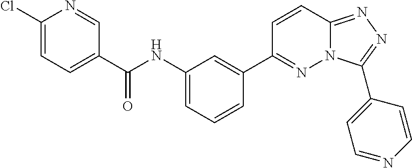 [SMILES: O=C(Nc1cccc(-c2ccc3nnc(-c4ccncc4)n3n2)c1)c1ccc(Cl)nc1]

or a salt thereof. In some embodiments, the compound having a structural Formula (III) is

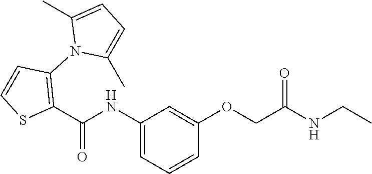 [SMILES: CCNC(=O)COc1cccc(NC(=O)c2sccc2-n2c(C)ccc2C)c1]

or a salt thereof. In some embodiments, the compound having a structural Formula (III) is

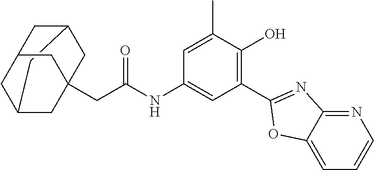 [SMILES: Cc1cc(NC(=O)CC23CC4CC(CC(C4)C2)C3)cc(-c2nc3ncccc3o2)c1O]

or a salt thereof. In some embodiments, the compound having a structural Formula (III) is

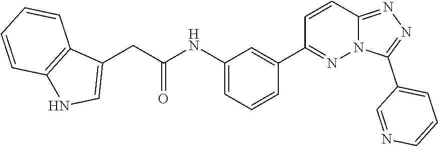 [SMILES: O=C(Cc1c[nH]c2ccccc12)Nc1cccc(-c2ccc3nnc(-c4cccnc4)n3n2)c1]

or a salt thereof. In some embodiments, the compound having a structural Formula (III) is

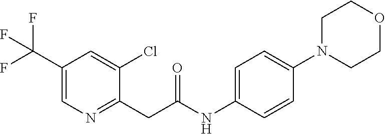 [SMILES: O=C(Cc1ncc(C(F)(F)F)cc1Cl)Nc1ccc(N2CCOCC2)cc1]

or a salt thereof. In some embodiments, the compound having a structural Formula (III) is

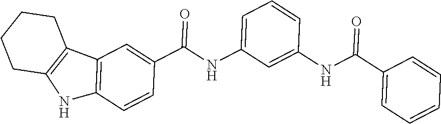 [SMILES: O=C(Nc1cccc(NC(=O)c2ccc3[nH]c4c(c3c2)CCCC4)c1)c1ccccc1]

or a salt thereof.

In certain aspects, disclosed herein is a compound represented by Formula (IV):

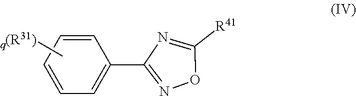

or a salt thereof,

wherein
each R³¹ is independently selected at each occurrence from hydrogen, halogen, —OR^{3a}, —CN, —NO₂, —N(R^{3a})₂, C₁₋₁₀ alkyl, —C₁₋₁₀ haloalkyl, —O—C₁₋₁₀ alkyl, C₂₋₁₀ alkenyl, C₂₋₁₀ alkynyl, optionally substituted C₃₋₁₂ carbocycle, and optionally substituted 3- to 12-membered heterocycle, wherein
R^{3a} is hydrogen or C₁-C₆ alkyl;
q is selected from 0, 1, or 2;
R⁴¹ is selected from hydrogen, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents selected from ═O, phenyl, and —C(O)R⁴², wherein R⁴² is selected from —OH, —CN, —NO₂, —NH₂, C₁₋₁₀ alkyl, optionally substituted C₃₋₁₂ carbocycle, and optionally substituted 3- to 12-membered heterocycle.

In some embodiments, R³¹ is hydrogen. In some embodiments, R³¹ is halogen. In some embodiments, R³¹ is —Cl. In some embodiments, R³¹ is —F. In some embodiments, R³¹ is —OR^{3a}. In some embodiments, R³¹ is —CN. In some embodiments, R³¹ is —N(R^{3a})₂. In some embodiments, R^{3a} is hydrogen. In some embodiments, R^{3a} is C₁-C₆ alkyl.

In some embodiments, R³¹ is optionally substituted 3- to 12-membered heterocycle. In some embodiments, R³¹ is optionally substituted 3-membered heterocycle. In some embodiments, R³¹ is optionally substituted 4-membered heterocycle. In some embodiments, R³¹ is optionally substituted 5-membered heterocycle. In some embodiments, R³¹ is optionally substituted 6-membered heterocycle. In some embodiments, R³¹ is optionally substituted 7-membered heterocycle. In some embodiments, R³¹ is optionally substituted 8-membered heterocycle. In some embodiments, R³¹ is optionally substituted 9-membered heterocycle. In some embodiments, R³¹ is optionally substituted 10-membered heterocycle. In some embodiments, R³¹ is optionally substituted 11-membered heterocycle. In some embodiments, R³¹ is optionally substituted 123-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, the heterocycle has three nitrogen atoms.

In some embodiments, R³¹ is selected from a triazole. In some embodiments, the triazole is optionally substituted with one or more R³². In some embodiments, the triazole is optionally substituted with one R³². In some embodiments, the triazole is optionally substituted with two R³². In some embodiments, each R³² is independently selected at each occurrence from halogen, C₁₋₆ alkyl, C₂₋₆ alkenyl, and C₂₋₆ alkynyl. In some embodiments, each R³² is independently selected at each occurrence from halogen and C₁₋₆ alkyl, C₂₋₆ alkenyl, and C₂₋₆ alkynyl. In some embodiments, each R³² is C₁ alkyl. In some embodiments, each R³² is C₂ alkyl. In some embodiments, each R³² is C₃ alkyl. In some embodiments, each R³² is C₄ alkyl. In some embodiments, each R³² is C₅ alkyl. In some embodiments, each R³² is C₆ alkyl. In some embodiments, R³¹ is

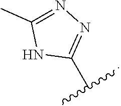 [SMILES: *c1nnc(C)[nH]1]

In some embodiments, q is 0 or 1. In some embodiments, q is 0. In some embodiments, q is 1.

In some embodiments, R⁴¹ is optionally substituted 3- to 12-membered carbocycle. In some embodiments, R⁴¹ is optionally substituted 3-membered carbocycle. In some embodiments, R⁴¹ is optionally substituted 4-membered carbocycle. In some embodiments, R⁴¹ is optionally substituted 5-membered carbocycle. In some embodiments, R⁴¹ is optionally substituted 6-membered carbocycle. In some embodiments, R⁴¹ is optionally substituted 7-membered carbocycle. In some embodiments, R⁴¹ is optionally substituted 8-membered carbocycle. In some embodiments, R⁴¹ is optionally substituted 9-membered carbocycle. In some embodiments, R⁴¹ is optionally substituted 10-membered carbocycle. In some embodiments, R⁴¹ is optionally substituted 11-membered carbocycle. In some embodiments, R⁴¹ is optionally substituted 12-membered carbocycle.

In some embodiments, R⁴¹ is optionally substituted 3- to 12-membered heterocycle. In some embodiments, R⁴¹ is optionally substituted 3-membered heterocycle. In some embodiments, R⁴¹ is optionally substituted 4-membered heterocycle. In some embodiments, R⁴¹ is optionally substituted 5-membered heterocycle. In some embodiments, R⁴¹ is optionally substituted 6-membered heterocycle. In some embodiments, R⁴¹ is optionally substituted 7-membered heterocycle. In some embodiments, R⁴¹ is optionally substituted 8-membered heterocycle. In some embodiments, R⁴¹ is optionally substituted 9-membered heterocycle. In some embodiments, R⁴¹ is optionally substituted 10-membered heterocycle. In some embodiments, R⁴¹ is optionally substituted 11-membered heterocycle. In some embodiments, R⁴¹ is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms.

In some embodiments, R⁴¹ is selected from

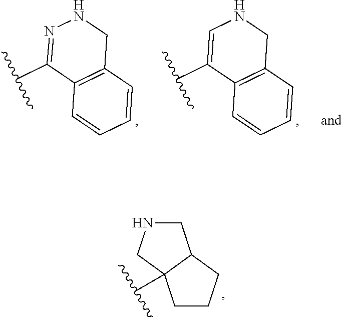

wherein each is optionally substituted with one or more substitutes. In some embodiments, R⁴¹ is optionally substituted

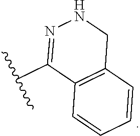 [SMILES: *C1=NNCc2ccccc21]

In some embodiments, R⁴¹ is optionally substituted

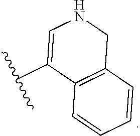 [SMILES: *C1=CNCc2ccccc21]

In some embodiments, R⁴¹ is optionally substituted

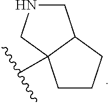 [SMILES: *C12CCCC1CNC2]

In some embodiments, the one or more substitutes are ═O. In some embodiments, the one or more substitutes are phenyl. In some embodiments, the one or more substitutes are —C(O)R⁴². In some embodiments, R⁴² is optionally substituted 3- to 12-membered heterocycle. In some embodiments, R⁴² is optionally substituted 3-membered heterocycle. In some embodiments, R⁴² is optionally substituted 4-membered heterocycle. In some embodiments, R⁴² is optionally substituted 5-membered heterocycle. In some embodiments, R⁴² is optionally substituted 6-membered heterocycle. In some embodiments, R⁴² is optionally substituted 7-membered heterocycle. In some embodiments, R⁴² is optionally substituted 8-membered heterocycle. In some embodiments, R⁴² is optionally substituted 9-membered heterocycle. In some embodiments, R⁴² is optionally substituted 10-membered heterocycle. In some embodiments, R⁴² is optionally substituted 11-membered heterocycle. In some embodiments, R⁴² is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, R⁴² is

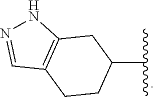 [SMILES: *C1CCc2cn[nH]c2C1]

In some embodiments, R⁴¹ is selected from

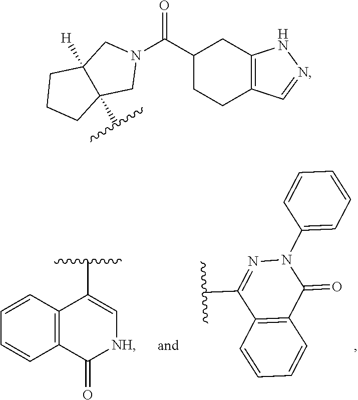 [SMILES: *[C@]12CCC[C@H]1CN(C(=O)C1CCc3cn[nH]c3C1)C2 ; *c1c[nH]c(=O)c2ccccc12 ; *c1nn(-c2ccccc2)c(=O)c2ccccc12]

In some embodiments, R⁴¹ is

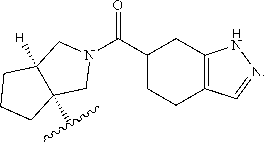 [SMILES: *[C@]12CCC[C@H]1CN(C(=O)C1CCc3cn[nH]c3C1)C2]

In some embodiments, R⁴¹ is

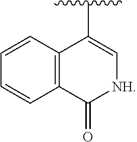 [SMILES: *c1c[nH]c(=O)c2ccccc12]

In some embodiments, R⁴¹ is

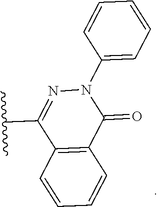 [SMILES: *c1nn(-c2ccccc2)c(=O)c2ccccc12]

In some embodiments, a compound having structural Formula (IV) is selected from those set forth in Table 4, and a salt thereof.

TABLE 4
Example Compounds of Formula (IV)

| ID # | Chemical Structure | Chemical Name |
|---|---|---|
| IV-1 | 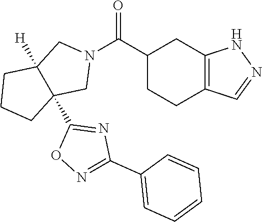 [SMILES: O=C(C1CCc2cn[nH]c2C1)N1C[C@@H]2CCC[C@]2(c2nc(-c3ccccc3)no2)C1] | 6-[(3aS,6aS)-3a-(3-phenyl- 1,2,4-oxadiazol-5-yl)- octahydrocyclopenta[c]pyrrole- 2-carbonyl]-4,5,6,7- tetrahydro-1H-indazole |
| IV-2 | 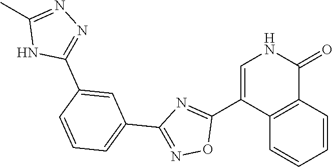 [SMILES: Cc1nnc(-c2cccc(-c3noc(-c4c[nH]c(=O)c5ccccc45)n3)c2)[nH]1] | 4-{3-[3-(5-methyl-4H-1,2,4- triazol-3-yl)phenyl]-1,2,4- oxadiazol-5-yl}-1,2- dihydroisoquinolin-1-one |
| IV-3 | 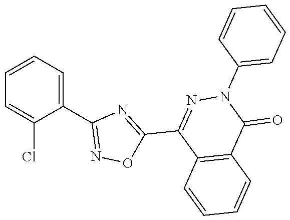 [SMILES: O=c1c2ccccc2c(-c2nc(-c3ccccc3Cl)no2)nn1-c1ccccc1] | 4-[3-(2-chlorophenyl)-1,2,4- oxadiazol-5-yl]-2-phenyl-1,2- dihydrophthalazin-1-one |

In some embodiments, the compound having a structural Formula (IV) is

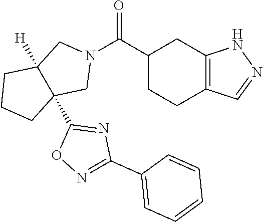 [SMILES: O=C(C1CCc2cn[nH]c2C1)N1C[C@@H]2CCC[C@]2(c2nc(-c3ccccc3)no2)C1]

or a salt thereof. In some embodiments, the compound having a structural Formula (IV) is

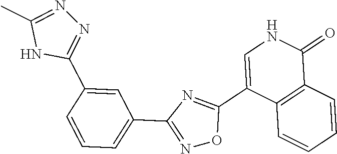 [SMILES: Cc1nnc(-c2cccc(-c3noc(-c4c[nH]c(=O)c5ccccc45)n3)c2)[nH]1]

or a salt thereof. In some embodiments, the compound having a structure Formula (IV) is

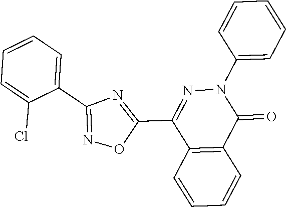 [SMILES: O=c1c2ccccc2c(-c2nc(-c3ccccc3Cl)no2)nn1-c1ccccc1]

or a salt thereof.

In certain aspects, disclosed herein is a compound represented by Formula (V):

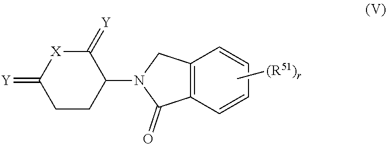

or a salt thereof,

wherein
X is selected from O, S, and NH;
Y is selected from O, S, and NH;
each R⁵¹ is independently selected at each occurrence from halogen, —OR⁵², —CN, —NO₂, —NR⁵²₂, C₁₋₁₀ alkyl, —C₁₋₁₀ haloalkyl, —OC(O)R⁵², —OCH₂C(O)R⁵², C₃₋₁₂ carbocycle, and optionally substituted 3- to 12-membered heterocycle;
   R⁵² is selected from hydrogen, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents selected from ═O, phenyl, —OH and optionally substituted 3- to 12-membered heterocycle; and
r is selected from 0, 1, 2, or 3.

In some embodiments, X is O. In some embodiments, X is S. In some embodiments, X is NH.

In some embodiments, Y is O. In some embodiments, Y is S. In some embodiments, Y is NH.

In some embodiments, each R⁵¹ is independently selected at each occurrence from —OR⁵², —OCH₂C(O)R⁵², and optionally substituted 3- to 12-membered heterocycle.

In some embodiments, R⁵¹ is —OR⁵². In some embodiments, R⁵² is hydrogen. In some embodiments, R⁵² is selected from —C₁₋₆ alkyl. In some embodiments, R⁵² is —C₁ alkyl. In some embodiments, R⁵² is —C₂ alkyl. In some embodiments, R⁵² is —C₃ alkyl. In some embodiments, R⁵² is —C₄ alkyl. In some embodiments, R⁵² is —C₅ alkyl. In some embodiments, R⁵² is —C₆ alkyl. In some embodiments, R⁵¹ is —OH. In some embodiments, R⁵¹ is —OCH₃. In some embodiments, R⁵¹ is —OCH₂CH₃. In some embodiments, R⁵¹ is —OCH₂CH₂CH₃.

In some embodiments, R⁵¹ is —OCH₂C(O)R⁵². In some embodiments, R⁵² is optionally substituted 3- to 12-membered heterocycle. In some embodiments, R⁵² is optionally substituted 3-membered heterocycle. In some embodiments, R⁵² is optionally substituted 4-membered heterocycle. In some embodiments, R⁵² is optionally substituted 5-membered heterocycle. In some embodiments, R⁵² is optionally substituted 6-membered heterocycle. In some embodiments, R⁵² is optionally substituted 7-membered heterocycle. In some embodiments, R⁵² is optionally substituted 8-membered heterocycle. In some embodiments, R⁵² is optionally substituted 9-membered heterocycle. In some embodiments, R⁵² is optionally substituted 10-membered heterocycle. In some embodiments, R⁵² is optionally substituted 11-membered heterocycle. In some embodiments, R⁵² is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, R⁵² is selected from

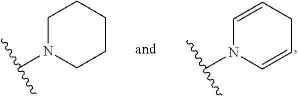 [SMILES: *N1CCCCC1 ; *N1C=CCC=C1]

optionally substituted with one or more substitutes.

In some embodiments, R⁵² is optionally substituted with one substituent selected from —OH and 3- to 12-membered heterocycle. In some embodiments, R⁵² is optionally substituted with —OH. In some embodiments, R⁵² is optionally substituted with 3-membered heterocycle. In some embodiments, R⁵² is optionally substituted with 4-membered heterocycle. In some embodiments, R⁵² is optionally substituted with 5-membered heterocycle. In some embodiments, R⁵² is optionally substituted with 6-membered heterocycle. In some embodiments, R⁵² is optionally substituted with 7-membered heterocycle. In some embodiments, R⁵² is optionally substituted with 8-membered heterocycle. In some embodiments, R⁵² is optionally substituted with 9-membered heterocycle. In some embodiments, R⁵² is optionally substituted with 10-membered heterocycle. In some embodiments, R⁵² is optionally substituted with 11-membered heterocycle. In some embodiments, R⁵² is optionally substituted with 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom.

In some embodiments, R⁵² is selected from

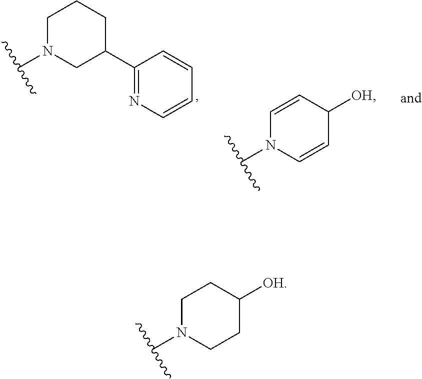 [SMILES: *N1CCCC(c2ccccn2)C1 ; *N1C=CC(O)C=C1 ; *N1CCC(O)CC1]

In some embodiments, R⁵² is

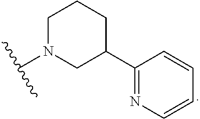 [SMILES: *N1CCCC(c2ccccn2)C1]

In some embodiments, R⁵² is

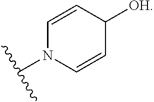 [SMILES: *N1C=CC(O)C=C1]

In some embodiments, R⁵² is

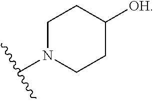 [SMILES: *N1CCC(O)CC1]

In some embodiments, R⁵¹ is selected from

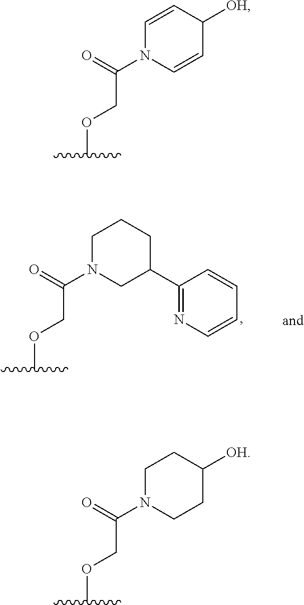 [SMILES: *OCC(=O)N1C=CC(O)C=C1 ; *OCC(=O)N1CCCC(c2ccccn2)C1 ; *OCC(=O)N1CCC(O)CC1]

In some embodiments, R⁵¹ is

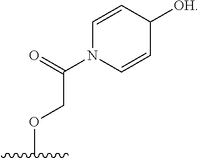 [SMILES: *OCC(=O)N1C=CC(O)C=C1]

In some embodiments, R⁵¹ is

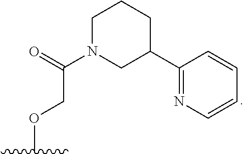 [SMILES: *OCC(=O)N1CCCC(c2ccccn2)C1]

In some embodiments, R⁵¹ is

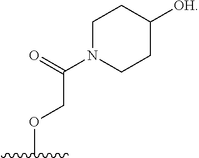 [SMILES: *OCC(=O)N1CCC(O)CC1]

In some embodiments, R⁵¹ is optionally substituted 3- to 12-membered heterocycle. In some embodiments, R⁵¹ is optionally substituted 3-membered heterocycle. In some embodiments, R⁵¹ is optionally substituted 6-membered heterocycle. In some embodiments, R⁵¹ is optionally substituted 4-membered heterocycle. In some embodiments, R⁵¹ is optionally substituted 5-membered heterocycle. In some embodiments, R⁵¹ is optionally substituted 6-membered heterocycle. In some embodiments, R⁵¹ is optionally substituted 7-membered heterocycle. In some embodiments, R⁵¹ is optionally substituted 8-membered heterocycle. In some embodiments, R⁵¹ is optionally substituted 9-membered heterocycle. In some embodiments, R⁵¹ is optionally substituted 10-membered heterocycle. In some embodiments, R⁵¹ is optionally substituted 11-membered heterocycle. In some embodiments, R⁵¹ is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom.

In some embodiments, R⁵¹ is selected from optionally substituted pyridine, tetrahydropyran, pyridazine, pyrimidine, and pyrazine. In some embodiments, R⁵¹ is selected from optionally substituted pyridine. In some embodiments, R⁵¹ is selected from optionally substituted tetrahydropyran. In some embodiments, R⁵¹ is selected from optionally substituted pyridazine. In some embodiments, R⁵¹ is selected from optionally substituted pyrimidine. In some embodiments, R⁵¹ is selected from optionally substituted pyrazine. In some embodiments, R⁵¹ is

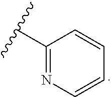 [SMILES: *c1ccccn1]

In some embodiments, r is 1 or 2. In some embodiments, r is 1. In some embodiments, r is 2. In some embodiments, r is 0. In some embodiments, r is 3.

In some embodiments, a compound having structural Formula (V) is selected from those set forth in Table 5, and a salt thereof.

TABLE 5
Example Compounds of Formula (V)

| ID # | Chemical Structure | Chemical Name |
|---|---|---|
| V-1 | 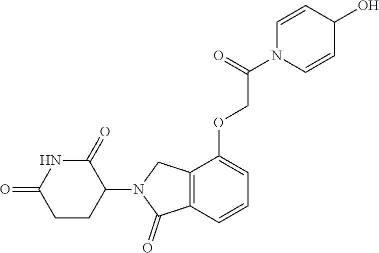 [SMILES: O=C1CCC(N2Cc3c(OCC(=O)N4C=CC(O)C=C4)cccc3C2=O)C(=O)N1] | 3-(4-(2-(4- hydroxypyridin-1(4H)- yl)-2-oxoethoxy)-1- oxoisoindolin-2- yl)piperidine-2,6-dione |
| V-2 | 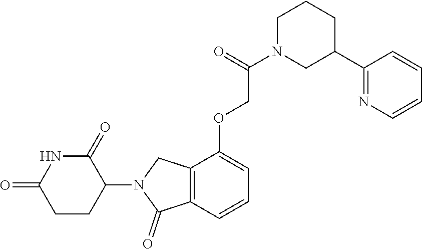 [SMILES: O=C1CCC(N2Cc3c(OCC(=O)N4CCCC(c5ccccn5)C4)cccc3C2=O)C(=O)N1] | 3-(1-oxo-4-{2-oxo-2-[3- (pyridin-2-yl)piperidin- 1-yl]ethoxy}-2,3- dihydro-1H-isoindol-2- yl)piperidine-2,6-dione |
| V-3 | 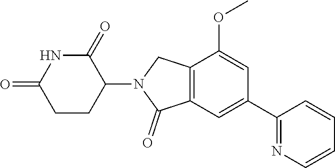 [SMILES: COc1cc(-c2ccccn2)cc2c1CN(C1CCC(=O)NC1=O)C2=O] | 3-(4-methoxy-1-oxo-6- (pyridin-2-yl)isoindolin- 2-yl)piperidine-2,6- dione |
| V-4 | 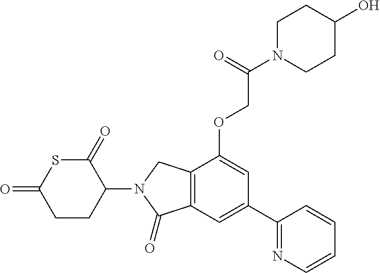 [SMILES: O=C1CCC(N2Cc3c(OCC(=O)N4CCC(O)CC4)cc(-c4ccccn4)cc3C2=O)C(=O)S1] | 3-(4-(2-(4- hydroxypiperidin-1-yl)- 2-oxoethoxy)-1-oxo-6- (pyridin-2-yl)isoindolin- 2-yl)dihydro-2H- thiopyran-2,6(3H)-dione |

In some embodiments, the compound having a structure of Formula (V) is

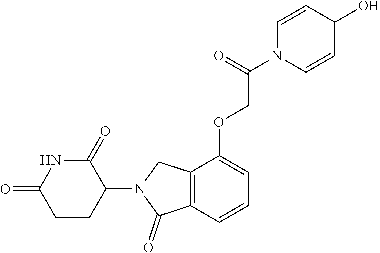 [SMILES: O=C1CCC(N2Cc3c(OCC(=O)N4C=CC(O)C=C4)cccc3C2=O)C(=O)N1]

or a salt thereof. In some embodiments, the compound having a structure of Formula (V) is

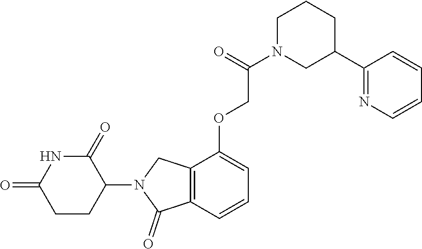 [SMILES: O=C1CCC(N2Cc3c(OCC(=O)N4CCCC(c5ccccn5)C4)cccc3C2=O)C(=O)N1]

or a salt thereof. In some embodiments, the compound having a structure of Formula (V) is

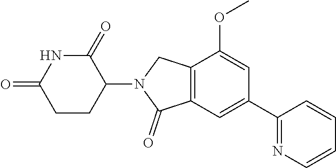 [SMILES: COc1cc(-c2ccccn2)cc2c1CN(C1CCC(=O)NC1=O)C2=O]

or a salt thereof. In some embodiments, the compound having a structure of Formula (V) is

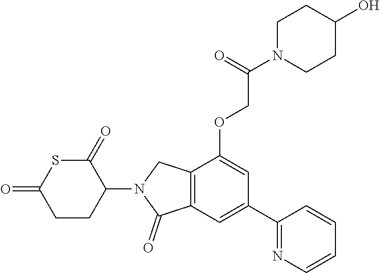 [SMILES: O=C1CCC(N2Cc3c(OCC(=O)N4CCC(O)CC4)cc(-c4ccccn4)cc3C2=O)C(=O)S1]

or a salt thereof.

In certain aspects, a compound disclosed herein is selected from those set forth in Table 6, and a salt thereof.

TABLE 6
Example Other Compounds

| ID # | Chemical Structure | Chemical Name |
|---|---|---|
| VI-1 | 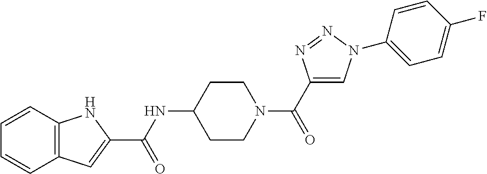 [SMILES: O=C(NC1CCN(C(=O)c2cn(-c3ccc(F)cc3)nn2)CC1)c1cc2ccccc2[nH]1] | N-{1-[1-(4-fluorophenyl)- 1H-1,2,3-triazole-4- carbonyl]piperidin-4-yl}-1H- indole-2- carboxamide |
| VI-2 | 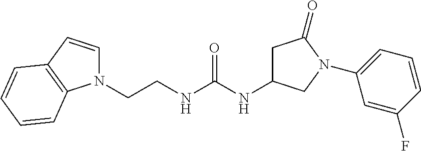 [SMILES: O=C(NCCn1ccc2ccccc21)NC1CC(=O)N(c2cccc(F)c2)C1] | 3-[1-(3-fluorophenyl)-5- oxopyrrolidin-3-y1]-1-[2- (1H-indol-1-yl)ethyl]urea |
| VI-3 | 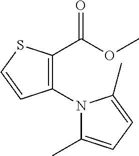 [SMILES: COC(=O)c1sccc1-n1c(C)ccc1C] | methyl 3-(2,5-dimethyl-1H- pyrrol-1-yl)thiophene-2- carboxylate |
| VI-4 | 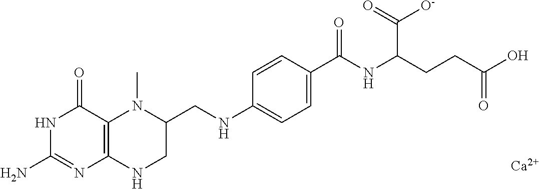 [SMILES: CN1c2c(nc(N)[nH]c2=O)NCC1CNc1ccc(C(=O)NC(CCC(=O)O)C(=O)[O-])cc1 ; [Ca+2]] | Levomefolate calcium |
| VI-5 | 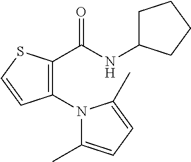 [SMILES: Cc1ccc(C)n1-c1ccsc1C(=O)NC1CCCC1] | N-cyclopenty1-3-(2,5- dimethy1-1H-pyrrol-1- yl)thiophene-2-carboxamide |
| VI-6 | 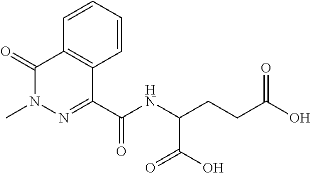 [SMILES: Cn1nc(C(=O)NC(CCC(=O)O)C(=O)O)c2ccccc2c1=O] | 2-[(3-methyl-4-oxo-3,4- dihydrophthalazin-1- yl)formamido]pentanedioic acid |
| VI-7 | 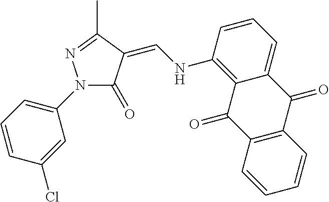 [SMILES: CC1=NN(c2cccc(Cl)c2)C(=O)/C1=C\Nc1cccc2c1C(=O)c1ccccc1C2=O] | 1-({[1-(3-chlorophenyl)-3- methyl-5-oxo-1,5-dihydro- 4H-pyrazol-4- ylidene]methyl}amino)anthra- 9,10-quinone |
| VI-8 | 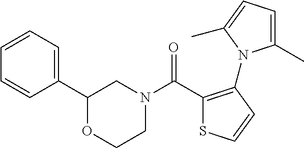 [SMILES: Cc1ccc(C)n1-c1ccsc1C(=O)N1CCOC(c2ccccc2)C1] | 4-[3-(2,5-dimethyl-1H- pyrrol-1-yl)thiophene-2- carbonyl]-2- phenylmorpholine |
| VI-9 | 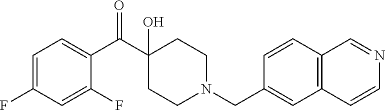 [SMILES: O=C(c1ccc(F)cc1F)C1(O)CCN(Cc2ccc3cnccc3c2)CC1] | 4-(2,4-difluorobenzoyl)-1- [(isoquinolin-6- yl)methyl]piperidin-4-ol |
| VI-10 | 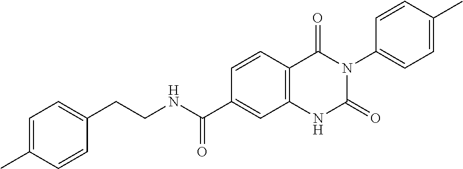 | 3-(4-methylpheny1)-N-[2-(4- methylphenyl)ethyl]-2,4- dioxo-1,2,3,4- tetrahydroquinazoline-7- carboxamide |
| VI-11 | 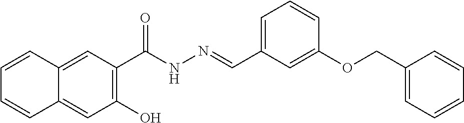 [SMILES: O=C(N/N=C/c1cccc(OCc2ccccc2)c1)c1cc2ccccc2cc1O] | N′-[3- (benzyloxy)benzylidene]-3- hydroxy-2-naphthohydrazide |
| VI-12 | 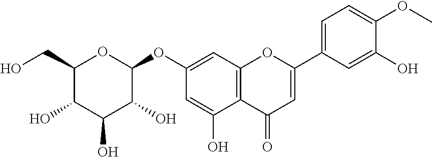 [SMILES: COc1ccc(-c2cc(=O)c3c(O)cc(O[C@@H]4O[C@H](CO)[C@@H](O)[C@H](O)[C@H]4O)cc3o2)cc1O] | Diosmetin-7-O-beta-D- glucopyranoside |
| VI-13 | 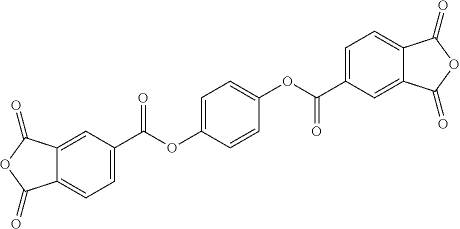 [SMILES: O=C(Oc1ccc(OC(=O)c2ccc3c(c2)C(=O)OC3=O)cc1)c1ccc2c(c1)C(=O)OC2=O] | 4-(1,3-dioxo-1,3-dihydro-2- benzofuran-5- carbonyloxy)phenyl 1,3- dioxo-1,3-dihydro-2- benzofuran-5-carboxylate |
| VI-14 | 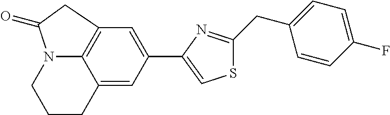 [SMILES: O=C1Cc2cc(-c3csc(Cc4ccc(F)cc4)n3)cc3c2N1CCC3] | 8-(2-(4-fluorobenzyl)thiazol- 4-yl)-5,6-dihydro-4H- pyrrolo[3,2,1-ij]quinolin- 2(1H)-one |

In some embodiments, a compound disclosed herein is selected from those (or any subset thereof) set forth in any one or combination of Tables 1 through 6, and salts thereof.

Compounds of the present disclosure may also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley and Sons, Inc., 1981, herein incorporated by reference for this disclosure). Stereoisomers may also be obtained by stereoselective synthesis.

The methods and compositions described herein may include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

Synthetic chemistry transformations and methodologies useful in synthesizing the compounds described herein are known in the art and include, for example, those described in R.

Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

Some compounds disclosed herein may include small molecules. A small molecule may include an organic compound of low molecular weight (e.g. 1000 daltons or less, or under 900 daltons).

### Methods

In certain aspects, the disclosure provides a method for treating, preventing, or providing a relief for a disease, disorder, or condition in a subject in need thereof with a composition comprising the compound(s) or salt(s) disclosed herein. In certain aspects, the disclosure provides a method for treating a disease, disorder, or condition in a subject in need thereof with a composition comprising the compound(s) or salt(s) disclosed herein. In certain aspects, the disclosure provides a method for providing a relief for a disease, disorder, or condition in a subject in need thereof with a composition comprising the compound(s) or salt(s) disclosed herein. In some embodiments, the method comprises administering the subject a composition comprising a compound or a salt thereof having a structural Formula of (I), (II), (III), (IV), (V), or Table 6. The compound(s) or salt(s) thereof may be selected from those set forth in any one of Tables 1-6, or any subset thereof, or any combination thereof. In some embodiments, the method comprises use, administration, or application of a compound having a structure of Formula (I), (II), (III), (IV), or (V), or a salt thereof. In some embodiments, the method comprises use, administration, or application of a compound having a structure in Table 1, Table 2, Table 3, Table 4, Table 5, or Table 6, or a salt thereof. In some embodiments, the composition comprises a cosmetic composition. In some embodiments, the composition comprises a pharmaceutical composition. In some embodiments, the composition comprises a cosmetically acceptable salt. In some embodiments, the composition comprises a pharmaceutically acceptable salt. In some embodiments, the composition comprises one or more compounds disclosed herein.

The disease, disorder, or condition comprises those characterized by abnormal or excess expression of IL-4 and/or IL-13, or by an abnormal host response to IL-4/IL-13 production. In some embodiments, the subject in need of an increased inflammatory response or in need of treatment according to the methods provided herein is a subject who has, is diagnosed as having, or is in need of treatment for asthma, allergies, cancer, atopic dermatitis, chronic rhinosinusitis with nasal polyposis (CRSwNP), eosinophilic esophagitis, an infection, or an autoimmune condition. In some embodiments, the disease, disorder, or condition of skin comprises eczema. In some embodiments, the compounds and a salt thereof disclosed herein are useful for treating a peripheral immune condition. In some embodiments, the compounds and a salt thereof disclosed herein are useful for treating an inflammatory condition. Beneficial or desired results of the administration of a composition comprising the compounds disclosed herein include, but are not limited to, a therapeutic benefit. In some embodiments, the administration of a composition comprising the compounds disclosed herein may reduce dryness, itching, redness, roughness, or flakiness of skin.

In some embodiments, the compounds or a salt thereof disclosed herein may inhibit IL-4 receptor signaling. In some embodiments, the compounds or salt thereof in the present disclosure may directly bind to IL-4 receptor and inhibit its downstream action. In some embodiments, the compound disclosed herein may result in a functional decrease in the expression of IL-4 and/or IL-13 in activated Th2 cells. In some embodiments, the compounds or a salt thereof disclosed herein may reduce the expression of IL-4 and IL-13 was tested in primary human Th2 cells. In some embodiments, the compound or salt of the present disclosure may not alter the proliferation or viability of healthy human dermal fibroblasts. In some embodiments, the compound or salt of the present disclosure may activate monocyte-derived dendritic cells (moDCs).

A diagnosis of condition, disorder, or disease may or may not have been made. In some embodiments of any method described herein, the subject may have been diagnosed with a condition, disorder, or disease. In some embodiments of any method described herein, the subject may not have been diagnosed with a condition, disorder, or disease.

In certain aspects, the present disclosure provides a method for treating, preventing, or providing a relief for a disease, disorder, or condition in a subject in need thereof, comprising administering the subject a composition comprising a compound having a structure of Formula (I) or salt thereof,

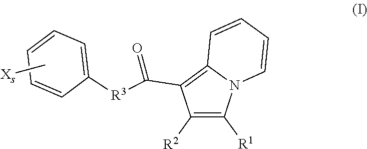

or a salt thereof,
wherein
R¹ is selected from optionally substituted C₁₋₆ alkyl, C₂₋₆ alkenyl, and C₂₋₆ alkynyl, —OR^{1a}, —SR^{1a}, —C(O)R^{1a}, —C(O)OR^{1a}, —OC(O)R^{1a}, —OC(O)N(R^{1a})₂, —NR^{1a}S(O)₂R^{1a}, —C(O)N(R^{1a})₂, —S(O)₂(R^{1a}), —S(O)₂N(R^{1a})₂, optionally substituted C₃-C₁₂ carbocycle, and optionally substituted 5- to 14-membered heterocycle;
   each R^{1a} is independently selected at each occurrence from C₁₋₆ alkyl, C₅-C₈ carbocycle, and C₅-C₈ heteroaryl, each of which is optionally substituted with one or more substituents selected from C₁₋₆ alkyl, halogen, —NO₂, —NR^{1b}₂, phenyl, —SR^{1b}, —OR^{1b}, and 3- to 12-membered heterocycle, wherein R^{1b} is hydrogen or C₁-C₆ alkyl;
R² is selected from hydrogen, —OH, halogen, C₁₋₆ alkoxy, C₁₋₆ haloalkoxy, —NH₂, and C₁₋₆ alkylamino;
R³ is selected from optionally substituted C₁₋₆ alkylene, optionally substituted C₁₋₆ haloalkyl, —CO—, —NR^{1b}—, and —O—;
each X is independently selected at each occurrence from —OR^{1b}, halogen, C₁₋₆ alkyl, or C₁₋₆ haloalkyl; and
s is selected from 0, 1, 2, and 3.

In some embodiments, R¹ is selected from optionally substituted C₁₋₆ alkyl, C₂₋₆ alkenyl, and C₂₋₆ alkynyl, —OR^{1a}, —SR^{1a}, —C(O)R^{1a}, and —C(O)OR^{1a}. In some embodiments, R¹ is optionally substituted C₁₋₆ alkyl. In some embodiments, R¹ is optionally substituted C₂₋₆ alkenyl. In some embodiments, R¹ is optionally substituted C₂₋₆ alkynyl. In some embodiments, R¹ is —SR^{1a}. In some embodiments, R¹ is —C(O)R^{1a}. In some embodiments, R¹ is —OC(O)R^{1a}. In some embodiments, R¹ is —OR^{1a}.

In some embodiments, R^{1a} is optionally substituted C₅-C₈ carbocycle. In some embodiments, R^{1a} is C₅-C₈ carbocycle optionally substituted with one or more substituents. In some embodiments, the one or more substituents are selected at each occurrence from C₁₋₆ alkyl, halogen, and —NO₂. In some embodiments, the one or more substituents are selected at each occurrence from C₁₋₆ alkyl. In some embodiments, the one or more substituents are selected at each occurrence from halogen. In some embodiments, the one or more substituents are and —NO₂. In some embodiments, R^{1a} is optionally substituted C₅ carbocycle or optionally substituted C₆ carbocycle. In some embodiments, R^{1a} is optionally substituted C₅ carbocycle. In some embodiments, R^{1a} is

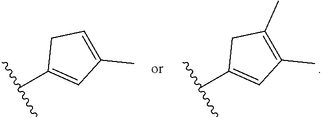

In some embodiments, R^{1a} is

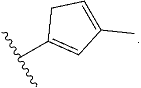 [SMILES: *C1=CC(C)=CC1]

In some embodiments, R^{1a} is

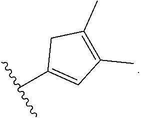 [SMILES: *C1=CC(C)=C(C)C1]

In some embodiments, R^{1a} is optionally substituted C₆ carbocycle. In some embodiments, R^{1a} is selected from

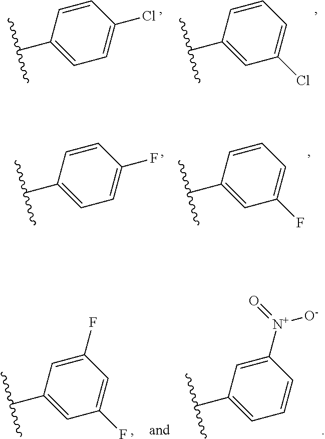 [SMILES: *c1ccc(Cl)cc1 ; *c1cccc(Cl)c1 ; *c1ccc(F)cc1 ; *c1cccc(F)c1 ; *c1cc(F)cc(F)c1 ; *c1cccc([N+](=O)[O-])c1]

In some embodiments, R^{1a} is

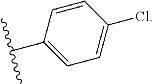 [SMILES: *c1ccc(Cl)cc1]

In some embodiments, R^{1a} is

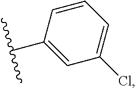 [SMILES: *c1cccc(Cl)c1]

In some embodiments, R^{1a} is

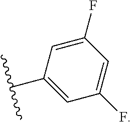 [SMILES: *c1cc(F)cc(F)c1]

In some embodiments, R^{1a} is

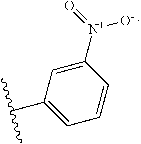 [SMILES: *c1cccc([N+](=O)[O-])c1]

In some embodiments, R^{1a} is

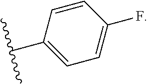 [SMILES: *c1ccc(F)cc1]

In some embodiments, R^{1a} is

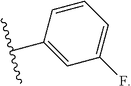 [SMILES: *c1cccc(F)c1]

In some embodiments, R² is hydrogen. In some embodiments, R² is halogen. In some embodiments, R² is —Cl. In some embodiments, R² is —F. In some embodiments, R² is —NH₂.

In some embodiments, R³ is selected from optionally substituted C₁₋₆ alkylene. In some embodiments, R³ is C₁ alkylene. In some embodiments, R³ is —CH₂—. In some embodiments, R³ is —NR^{1b}—. In some embodiments, R^{1b} is hydrogen. In some embodiments, R^{1b} is selected from C₁-C₆ alkyl. In some embodiments, R³ is —NH—.

In some embodiments, X is —OR^{1b}. In some embodiments, R^{1b} is hydrogen. In some embodiments, R^{1b} is selected from C₁-C₆ alkyl. In some embodiments, X is —OH. In some embodiments, X is —OCH₃. In some embodiments, X is halogen. In some embodiments, X is —Cl. In some embodiments, X is —F. In some embodiments, s is 1 or 2. In some embodiments, s is 1. In some embodiments, s is 2.

In some embodiments, the compound having a structure of Formula (I) is selected from the group consisting of:

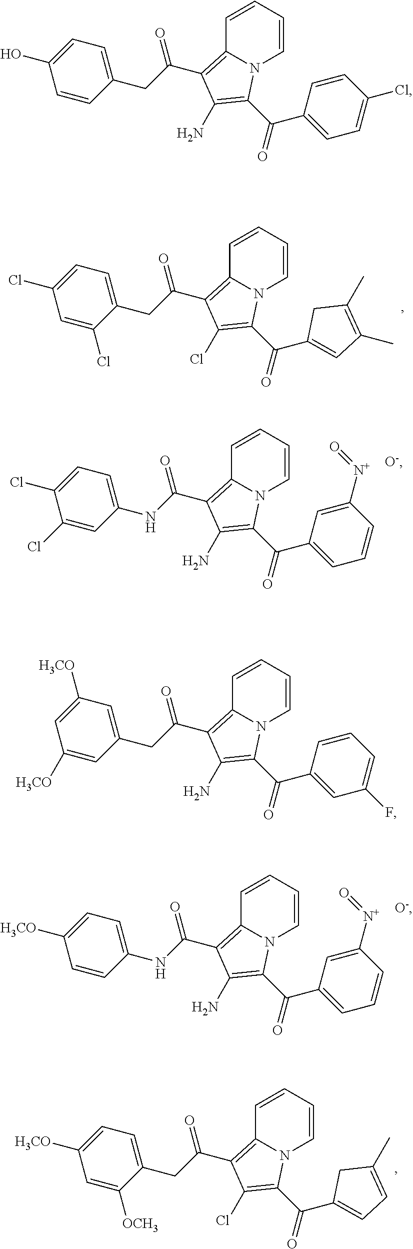

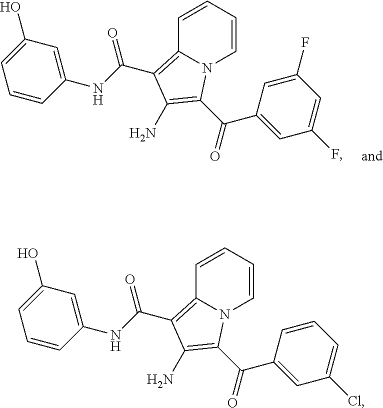 [SMILES: Nc1c(C(=O)Nc2cccc(O)c2)c2ccccn2c1C(=O)c1cc(F)cc(F)c1 ; Nc1c(C(=O)Nc2cccc(O)c2)c2ccccn2c1C(=O)c1cccc(Cl)c1]

and a salt thereof.

In certain aspects, the present disclosure provides a method for treating, preventing, or providing a relief for a disease, disorder, or condition in a subject in need thereof, comprising administering the subject a composition comprising a compound having a structure of Formula (II) or salt thereof,

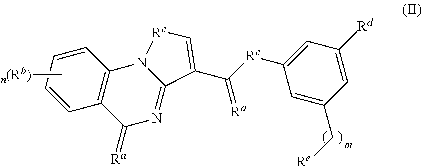

or a salt thereof,

wherein
each R^{a} is independently selected from O, S, and —NH;
each R^{b} is independently selected at each occurrence from hydrogen, —OR^{b1}, halogen, C₁₋₆ alkoxy, C₁₋₆ haloalkoxy, —NR^{b1}₂, and C₁₋₆ alkylamino;
each R^{c} is independently selected from O, S, or NH;
R^{d} is selected from hydrogen, —OR^{b1}, halogen, C₁₋₆ alkoxy, C₁₋₆ haloalkoxy, —NR^{b1}₂, and C₁₋₆ alkylamino;
R^{e} is selected from —C₁-C₆ alkyl, optionally substituted C₅-C₁₀ carbocycle, and optionally substituted C₅-C₁₀ heterocycle;
n is selected from 0, 1, 2, or 3; and
m is selected from 1, 2, or 3,
wherein R^{b1} is hydrogen or C₁-C₆ alkyl.

In some embodiments, R^{a} is O. In some embodiments, R^{a} is S. In some embodiments, R^{a} is NH.

In some embodiments, R^{b} is hydrogen. In some embodiments, R^{b} is —OR^{b1}. In some embodiments, R^{b1} is hydrogen. In some embodiments, R^{b1} is selected from C₁-C₆ alkyl. In some embodiments, R^{b} is —OH. In some embodiments, R^{b} is halogen. In some embodiments, R^{b} is —Cl. In some embodiments, R^{b} is —F.

In some embodiments, n is 0, 1, or 2. In some embodiments, n is 0. In some embodiments, n is 1. In some embodiments, n is 2.

In some embodiments, R^{c} is O. In some embodiments, R^{c} is S. In some embodiments, R^{c} is NH.

In some embodiments, R^{d} is selected from hydrogen, —OR^{b1}, and halogen. In some embodiments, R^{d} is hydrogen. In some embodiments, R^{d} is —OR^{b1}. In some embodiments, R^{b1} is hydrogen. In some embodiments, R^{b1} is selected from C₁-C₆ alkyl. In some embodiments, R^{d} is —OH. In some embodiments, R^{d} is halogen. In some embodiments, R^{d} is —Cl. In some embodiments, R^{d} is —F.

In some embodiments, R^{e} is optionally substituted C₅-C₁₀ carbocycle. In some embodiments, R^{e} is optionally substituted C₅-C₁₀ heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, the heterocycle has three nitrogen atoms. In some embodiments, R^{e} is selected from the group consisting of:

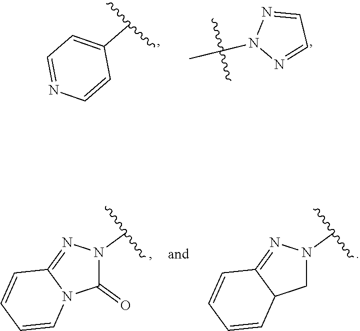 [SMILES: *c1ccncc1 ; *n1nccn1 ; *n1nc2ccccn2c1=O ; *N1CC2C=CC=CC2=N1]

In some embodiments, R^{e} is

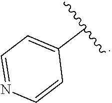 [SMILES: *c1ccncc1]

In some embodiments, R^{e} is

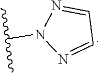 [SMILES: *n1nccn1]

In some embodiments, R^{e} is

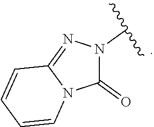 [SMILES: *n1nc2ccccn2c1=O]

In some embodiments, R^{e} is

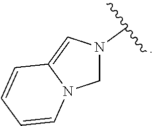 [SMILES: *N1C=C2C=CC=CN2C1]

In some embodiments, m is 1 or 2. In some embodiments, m is 1. In some embodiments, m is 2.

In some embodiments, the compound having a structure of Formula (II) is selected from the group consisting of:

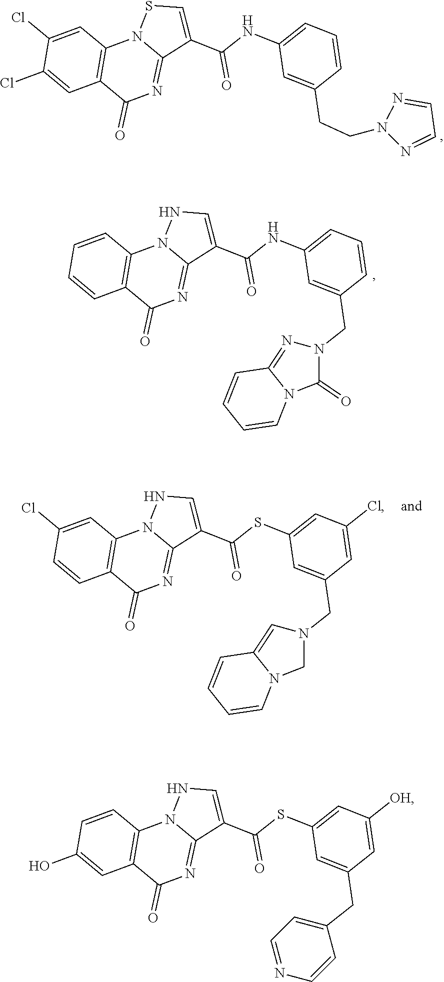 [SMILES: O=C(Nc1cccc(CCn2nccn2)c1)c1csn2c1nc(=O)c1cc(Cl)c(Cl)cc12 ; O=C(Nc1cccc(Cn2nc3ccccn3c2=O)c1)c1c[nH]n2c1nc(=O)c1ccccc12 ; O=C(Sc1cc(Cl)cc(CN2C=C3C=CC=CN3C2)c1)c1c[nH]n2c1nc(=O)c1ccc(Cl)cc12 ; O=C(Sc1cc(O)cc(Cc2ccncc2)c1)c1c[nH]n2c1nc(=O)c1cc(O)ccc12]

and a salt thereof.

In certain aspects, the present disclosure provides a method for treating, preventing, or providing a relief for a disease, disorder, or condition in a subject in need thereof, comprising administering the subject a composition comprising a compound having a structure of Formula (III) or salt thereof,

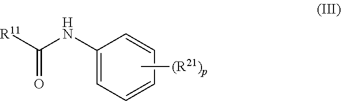

or a salt thereof,
R¹¹ is selected from —CH₂R¹², —OR¹², —SR¹², —C(O)R¹², —C(O)OR¹², —OC(O)R¹², —OC(O)N(R¹²)₂, —NR¹²S(O)₂R¹², —C(O)N(R¹²)₂, —S(O)₂(R¹²), —S(O)₂N(R¹²)₂, optionally substituted C₃-C₁₂ carbocycle, and optionally substituted 5- to 14-membered heterocycle;
   each R¹² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle;
each R²¹ is independently selected at each occurrence from —OH, halogen, C₁₋₆ alkyl, C₁₋₆ haloalkyl, OR²², —SR²², —C(O)R²², —C(O)OR²², —OC(O)R²², —OCH₂C(O)R²², —OC(O)N(R²²)₂, —OCH₂C(O)NHR²², —OCH₂C(O)N(R²²)₂, —NR²²S(O)₂R²², —C(O)N(R²²)₂, —S(O)₂(R²²), —S(O)₂NHR²², —S(O)₂N(R²²)₂, —NHC(O)R²², optionally substituted C₃-C₁₂ carbocycle, and optionally substituted 5- to 14-membered heterocycle;
   each R²² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle; and p is selected from 0, 1, 2, 3, or 4.

In some embodiments, the compound has a structure of Formula (III-1):

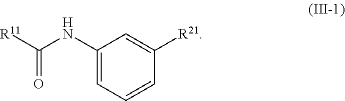

In some embodiments, the compound has a structure of Formula (III-2):

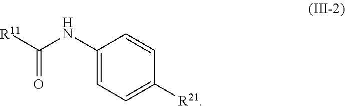

In some embodiments, the compound has a structure of Formula (III-3):

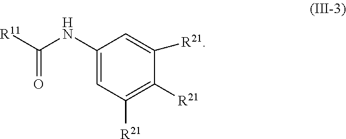

In some embodiments, R¹¹ is —CH₂R¹². In some embodiments, R¹² is optionally substituted C₃-C₁₂ carbocycle or optionally substituted 5- to 14-membered heterocycle. In some embodiments, R¹² is selected from optionally substituted C₃-C₁₂ carbocycle. In some embodiments, R¹² is selected from optionally substituted C₃ carbocycle. In some embodiments, R¹² is selected from optionally substituted C₃ carbocycle. In some embodiments, R¹² is selected from optionally substituted C₄ carbocycle. In some embodiments, R¹² is selected from optionally substituted C₅ carbocycle. In some embodiments, R¹² is selected from optionally substituted C₆ carbocycle. In some embodiments, R¹² is selected from optionally substituted C₇ carbocycle. In some embodiments, R¹² is selected from optionally substituted C₈ carbocycle. In some embodiments, R¹² is selected from optionally substituted C₉ carbocycle. In some embodiments, R¹² is selected from optionally substituted C₁₀ carbocycle. In some embodiments, R¹² is selected from optionally substituted C₁₁ carbocycle. In some embodiments, R¹² is selected from optionally substituted C₁₂ carbocycle. In some embodiments, R¹² is selected from optionally substituted 5- to 14-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, the heterocycle has three nitrogen atoms. In some embodiments, R¹² is selected from optionally substituted 5-membered heterocycle. In some embodiments, R¹² is selected from optionally substituted 6-membered heterocycle. In some embodiments, R¹² is selected from optionally substituted 7-membered heterocycle. In some embodiments, R¹² is selected from optionally substituted 8-membered heterocycle. In some embodiments, R¹² is selected from optionally substituted 9-membered heterocycle. In some embodiments, R¹² is selected from optionally substituted 10-membered heterocycle. In some embodiments, R¹² is selected from optionally substituted 11-membered heterocycle. In some embodiments, R¹² is selected from optionally substituted 12-membered heterocycle. In some embodiments, R¹² is selected from optionally substituted 13-membered heterocycle. In some embodiments, R¹² is selected from optionally substituted 14-membered heterocycle. In some embodiments, R¹² is selected from

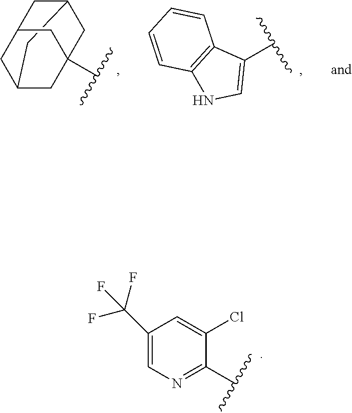 [SMILES: *C12CC3CC(CC(C3)C1)C2 ; *c1c[nH]c2ccccc12 ; *c1ncc(C(F)(F)F)cc1Cl]

In some embodiments, R¹² is

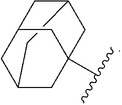 [SMILES: *C12CC3CC(CC(C3)C1)C2]

In some embodiments, R¹² is

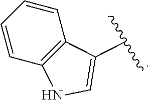 [SMILES: *c1c[nH]c2ccccc12]

In some embodiments, R¹² is

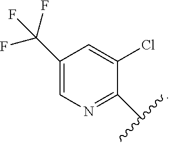 [SMILES: *c1ncc(C(F)(F)F)cc1Cl]

In some embodiments, R¹¹ is selected from

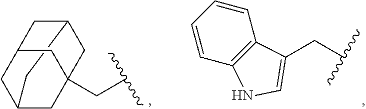 [SMILES: *CC12CC3CC(CC(C3)C1)C2 ; *Cc1c[nH]c2ccccc12]

and

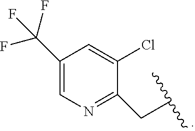 [SMILES: *Cc1ncc(C(F)(F)F)cc1Cl]

In some embodiments, R¹¹ is

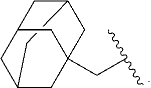 [SMILES: *CC12CC3CC(CC(C3)C1)C2]

In some embodiments, R¹¹ is

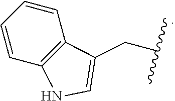 [SMILES: *Cc1c[nH]c2ccccc12]

In some embodiments, R¹¹ is

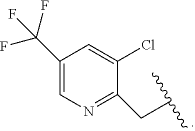 [SMILES: *Cc1ncc(C(F)(F)F)cc1Cl]

In some embodiments, R¹¹ is —OR¹². In some embodiments, R¹¹ is —OH. In some embodiments, R¹¹ is —SR¹². In some embodiments, R¹¹ is optionally substituted C₃-C₁₂ carbocycle or optionally substituted 5- to 14-membered heterocycle. In some embodiments, R¹¹ is selected from optionally substituted C₃-C₁₂ carbocycle. In some embodiments, R¹¹ is selected from optionally substituted C₃ carbocycle. In some embodiments, R¹¹ is selected from optionally substituted C₄ carbocycle. In some embodiments, R¹¹ is selected from optionally substituted C₅ carbocycle. In some embodiments, R¹¹ is selected from optionally substituted C₆ carbocycle. In some embodiments, R¹¹ is selected from optionally substituted C₇ carbocycle. In some embodiments, R¹¹ is selected from optionally substituted C₈ carbocycle. In some embodiments, R¹¹ is selected from optionally substituted C₉ carbocycle. In some embodiments, R¹¹ is selected from optionally substituted C₁₀ carbocycle. In some embodiments, R¹¹ is selected from optionally substituted C₁₁ carbocycle. In some embodiments, R¹¹ is selected from optionally substituted C₁₂ carbocycle. In some embodiments, R¹¹ is selected from optionally substituted 5- to 14-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has at least one sulfur atom. In some embodiments, the heterocycle has a sulfur atom. In some embodiments, R¹¹ is optionally substituted 5-membered heterocycle. In some embodiments, R¹¹ is optionally substituted 6-membered heterocycle. In some embodiments, R¹¹ is optionally substituted 7-membered heterocycle. In some embodiments, R¹¹ is optionally substituted 8-membered heterocycle. In some embodiments, R¹¹ is optionally substituted 9-membered heterocycle. In some embodiments, R¹¹ is optionally substituted 10-membered heterocycle. In some embodiments, R¹¹ is optionally substituted 11-membered heterocycle. In some embodiments, R¹¹ is optionally substituted 12-membered heterocycle. In some embodiments, R¹¹ is optionally substituted 13-membered heterocycle. In some embodiments, R¹¹ is optionally substituted 14-membered heterocycle. In some embodiments, R¹¹ is

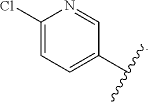 [SMILES: *c1ccc(Cl)nc1]

In some embodiments, R¹¹ is

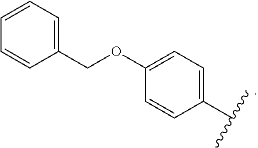 [SMILES: *c1ccc(OCc2ccccc2)cc1]

In some embodiments, R¹¹ is

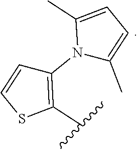 [SMILES: *c1sccc1-n1c(C)ccc1C]

In some embodiments, R¹¹ is

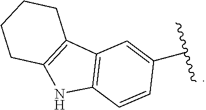 [SMILES: *c1ccc2[nH]c3c(c2c1)CCCC3]

In some embodiments, R²¹ is —OH. In some embodiments, R²¹ is C₁₋₆ alkyl. In some embodiments, R²¹ is C₁ alkyl. In some embodiments, R²¹ is C₂ alkyl. In some embodiments, R²¹ is C₃ alkyl. In some embodiments, R²¹ is C₄ alkyl. In some embodiments, R²¹ is C₅ alkyl. In some embodiments, R²¹ is C₆ alkyl.

In some embodiments, R²¹ is —OCH₂C(O)NHR²². In some embodiments, R²² is C₁ alkyl. In some embodiments, R²² is C₂alkyl. In some embodiments, R²² is C₃ alkyl. In some embodiments, R²² is C₄ alkyl. In some embodiments, R²¹ is —OCH₂C(O)NHCH₃. In some embodiments, R²¹ is —OCH₂C(O)NHCH₂CH₃. In some embodiments, R²¹ is —OCH₂C(O)NHCH₂CH₂CH₃.

In some embodiments, R²¹ is —S(O)₂NHR²². In some embodiments, R²² is optionally substituted 5- to 14-membered heterocycle. In some embodiments, R²² is optionally substituted 5-membered heterocycle. In some embodiments, R²² is optionally substituted 6-membered heterocycle. In some embodiments, R²² is optionally substituted 7-membered heterocycle. In some embodiments, R²² is optionally substituted 8-membered heterocycle. In some embodiments, R²² is optionally substituted 9-membered heterocycle. In some embodiments, R²² is optionally substituted 10-membered heterocycle. In some embodiments, R²² is optionally substituted 11-membered heterocycle. In some embodiments, R²² is optionally substituted 12-membered heterocycle. In some embodiments, R²² is optionally substituted 13-membered heterocycle. In some embodiments, R²² is optionally substituted 14-membered heterocycle. In some embodiments, R²² is

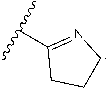 [SMILES: *C1=NCCC1]

In some embodiments, R²¹ is

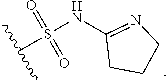 [SMILES: *S(=O)(=O)NC1=NCCC1]

In some embodiments, R²¹ is —NHC(O)R²². In some embodiments, R²² is selected from optionally substituted C₃-C₁₂ carbocycle. In some embodiments, R²² is optionally substituted C₃ carbocycle. In some embodiments, R²² is optionally substituted C₄ carbocycle. In some embodiments, R²² is optionally substituted C₅ carbocycle. In some embodiments, R²² is optionally substituted C₆ carbocycle. In some embodiments, R²² is optionally substituted C₇ carbocycle. In some embodiments, R²² is optionally substituted C₈ carbocycle. In some embodiments, R²² is optionally substituted C₉ carbocycle. In some embodiments, R²² is optionally substituted C₁₀ carbocycle. In some embodiments, R²² is optionally substituted C₁₁ carbocycle. In some embodiments, R²² is optionally substituted C₁₂ carbocycle. In some embodiments, R²¹ is optionally substituted 5- to 14-membered heterocycle. In some embodiments, R²¹ is optionally substituted 5-membered heterocycle. In some embodiments, R²¹ is optionally substituted 6-membered heterocycle. In some embodiments, R²¹ is optionally substituted 7-membered heterocycle. In some embodiments, R²¹ is optionally substituted 8-membered heterocycle. In some embodiments, R²¹ is optionally substituted 9-membered heterocycle. In some embodiments, R²¹ is optionally substituted 10-membered heterocycle. In some embodiments, R²¹ is optionally substituted 11-membered heterocycle. In some embodiments, R²¹ is optionally substituted 12-membered heterocycle. In some embodiments, R²¹ is optionally substituted 13-membered heterocycle. In some embodiments, R²¹ is optionally substituted 14-membered heterocycle. In some embodiments, R²¹ is

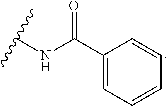 [SMILES: *NC(=O)c1ccccc1]

In some embodiments, R²¹ is optionally substituted 5- to 14-membered heterocycle. In some embodiments, R²¹ is selected at each occurrence from

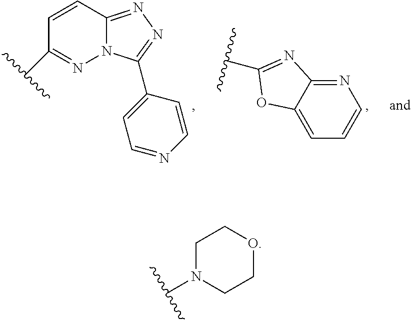 [SMILES: *c1ccc2nnc(-c3ccncc3)n2n1 ; *c1nc2ncccc2o1 ; *N1CCOCC1]

In some embodiments, R²¹ is selected from —OH, —CH₃,

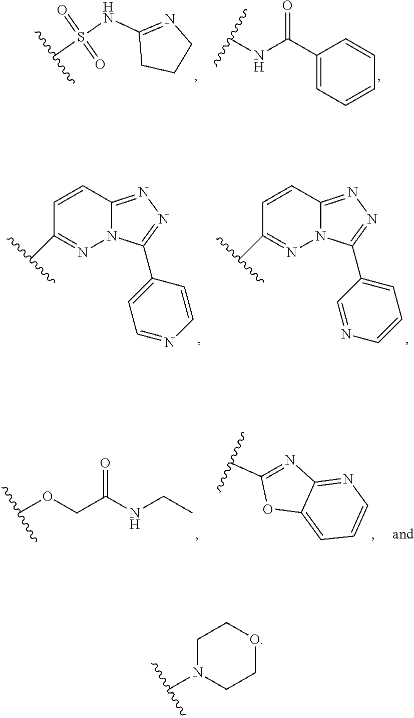

In some embodiments, R²¹ is —OH. In some embodiments, R²¹ is —CH₃. In some embodiments, R²¹ is

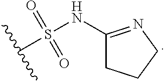 [SMILES: *S(=O)(=O)NC1=NCCC1]

In some embodiments, R²¹ is

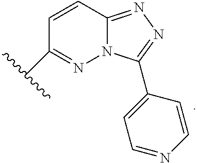 [SMILES: *c1ccc2nnc(-c3ccncc3)n2n1]

In some embodiments, R²¹ is

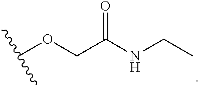 [SMILES: *OCC(=O)NCC]

In some embodiments, R²¹ is

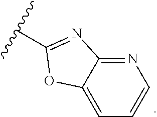 [SMILES: *c1nc2ncccc2o1]

In some embodiments, R²¹ is

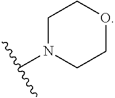 [SMILES: *N1CCOCC1]

In some embodiments, R²¹ is

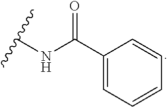 [SMILES: *NC(=O)c1ccccc1]

In some embodiments, R²¹ is

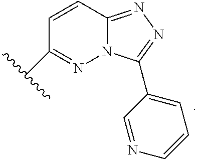 [SMILES: *c1ccc2nnc(-c3cccnc3)n2n1]

In some embodiments, p is selected from 1, 2, and 3. In some embodiments, p is 1. In some embodiments, p is 2. In some embodiments, p is 3.

In some embodiments, the compound having a structure of Formula (III) is selected from the group consisting of:

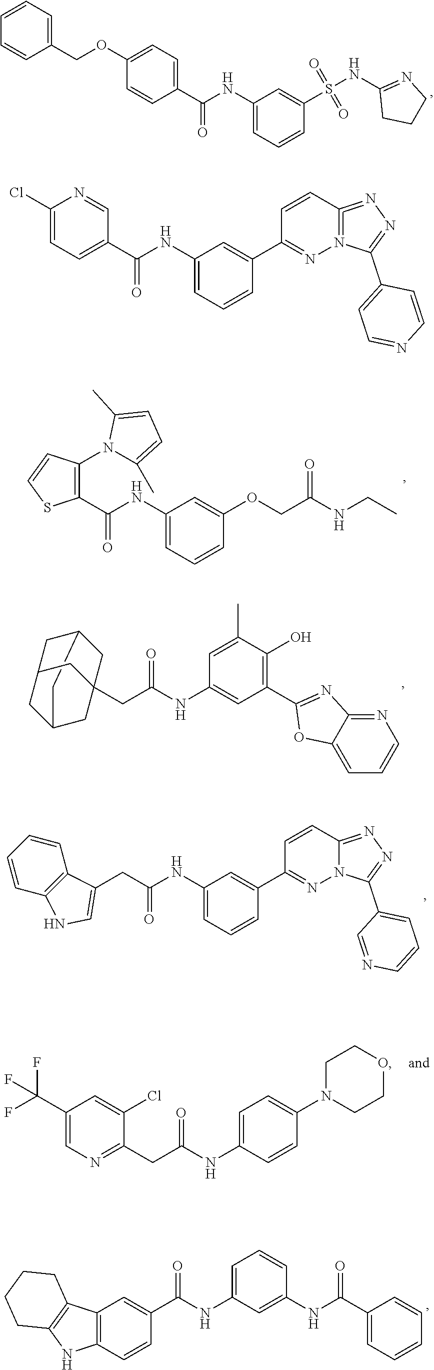

and a salt thereof.

In some embodiments, the compound is

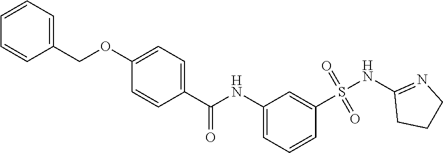 [SMILES: O=C(Nc1cccc(S(=O)(=O)NC2=NCCC2)c1)c1ccc(OCc2ccccc2)cc1]

or a salt thereof. In some embodiments, the compound is

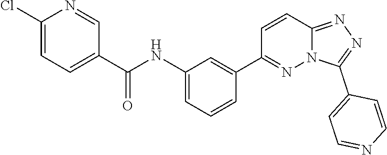 [SMILES: O=C(Nc1cccc(-c2ccc3nnc(-c4ccncc4)n3n2)c1)c1ccc(Cl)nc1]

or a salt thereof. In some embodiments, the compound is

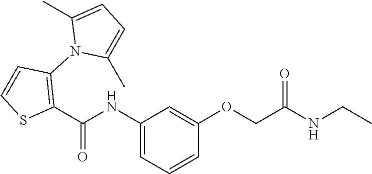 [SMILES: CCNC(=O)COc1cccc(NC(=O)c2sccc2-n2c(C)ccc2C)c1]

or a salt thereof. In some embodiments, the compound is

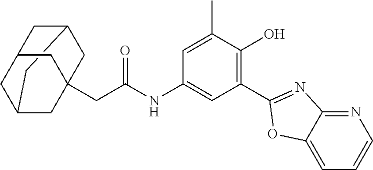 [SMILES: Cc1cc(NC(=O)CC23CC4CC(CC(C4)C2)C3)cc(-c2nc3ncccc3o2)c1O]

or a salt thereof. In some embodiments, the compound is

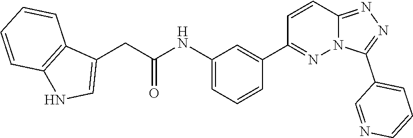 [SMILES: O=C(Cc1c[nH]c2ccccc12)Nc1cccc(-c2ccc3nnc(-c4cccnc4)n3n2)c1]

or a salt thereof. In some embodiments, the compound is

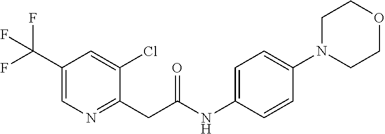 [SMILES: O=C(Cc1ncc(C(F)(F)F)cc1Cl)Nc1ccc(N2CCOCC2)cc1]

or a salt thereof. In some embodiments, the compound is

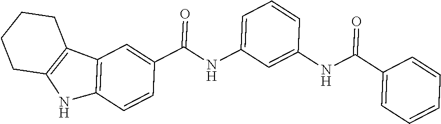 [SMILES: O=C(Nc1cccc(NC(=O)c2ccc3[nH]c4c(c3c2)CCCC4)c1)c1ccccc1]

or a salt thereof.

In certain aspects, the present disclosure provides a method for treating, preventing, or providing a relief for a disease, disorder, or condition in a subject in need thereof, comprising administering the subject a composition comprising a compound having a structure of Formula (IV) or salt thereof,

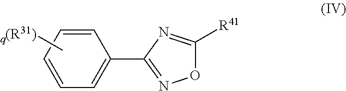

or a salt thereof,

wherein
each R³¹ is independently selected at each occurrence from hydrogen, halogen, —OR^{3a}, —CN, —NO₂, —N(R^{3a})₂, C₁₋₁₀ alkyl, —C₁₋₁₀ haloalkyl, —O—C₁₋₁₀ alkyl, C₂₋₁₀ alkenyl, C₂₋₁₀ alkynyl, optionally substituted C₃₋₁₂ carbocycle, and optionally substituted 3- to 12-membered heterocycle, wherein
R^{3a} is hydrogen or C₁-C₆ alkyl;
q is selected from 0, 1, or 2;
R⁴¹ is selected from hydrogen, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents selected from ═O, phenyl, —C(O)R⁴², wherein R⁴² is selected from —OH, —CN, —NO₂, —NH₂, C₁₋₁₀ alkyl, optionally substituted C₃₋₁₂ carbocycle, and optionally substituted 3- to 12-membered heterocycle.

In some embodiments, R³¹ is hydrogen. In some embodiments, R³¹ is halogen. In some embodiments, R³¹ is —Cl. In some embodiments, R³¹ is —F. In some embodiments, R³¹ is —OR^{3a}. In some embodiments, R³¹ is —CN. In some embodiments, R³¹ is —N(R^{3a})₂. In some embodiments, R^{3a} is hydrogen. In some embodiments, R^{3a} is C₁-C₆ alkyl.

In some embodiments, R³¹ is optionally substituted 3- to 12-membered heterocycle. In some embodiments, R³¹ is optionally substituted 3-membered heterocycle. In some embodiments, R³¹ is optionally substituted 4-membered heterocycle. In some embodiments, R³¹ is optionally substituted 5-membered heterocycle. In some embodiments, R³¹ is optionally substituted 6-membered heterocycle. In some embodiments, R³¹ is optionally substituted 7-membered heterocycle. In some embodiments, R³¹ is optionally substituted 8-membered heterocycle. In some embodiments, R³¹ is optionally substituted 9-membered heterocycle. In some embodiments, R³¹ is optionally substituted 10-membered heterocycle. In some embodiments, R³¹ is optionally substituted 11-membered heterocycle. In some embodiments, R³¹ is optionally substituted 123-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, the heterocycle has three nitrogen atoms.

In some embodiments, R³¹ is selected from a triazole. In some embodiments, the triazole is optionally substituted with one or more R³². In some embodiments, the triazole is optionally substituted with one R³². In some embodiments, the triazole is optionally substituted with two R³². In some embodiments, each R³² is independently selected at each occurrence from halogen, C₁₋₆ alkyl, C₂₋₆ alkenyl, and C₂₋₆ alkynyl. In some embodiments, each R³² is independently selected at each occurrence from halogen and C₁₋₆ alkyl, C₂₋₆ alkenyl, and C₂₋₆ alkynyl. In some embodiments, each R³² is C₁ alkyl. In some embodiments, each R³² is C₂ alkyl. In some embodiments, each R³² is C₃ alkyl. In some embodiments, each R³² is C₄ alkyl. In some embodiments, each R³² is C₅ alkyl. In some embodiments, each R³² is C₆ alkyl. In some embodiments, R³¹ is

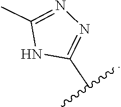 [SMILES: *c1nnc(C)[nH]1]

In some embodiments, q is 0 or 1. In some embodiments, q is 0. In some embodiments, q is 1.

In some embodiments, R⁴¹ is optionally substituted 3- to 12-membered carbocycle. In some embodiments, R⁴¹ is optionally substituted 3-membered carbocycle. In some embodiments, R⁴¹ is optionally substituted 4-membered carbocycle. In some embodiments, R⁴¹ is optionally substituted 5-membered carbocycle. In some embodiments, R⁴¹ is optionally substituted 6-membered carbocycle. In some embodiments, R⁴¹ is optionally substituted 7-membered carbocycle. In some embodiments, R⁴¹ is optionally substituted 8-membered carbocycle. In some embodiments, R⁴¹ is optionally substituted 9-membered carbocycle. In some embodiments, R⁴¹ is optionally substituted 10-membered carbocycle. In some embodiments, R⁴¹ is optionally substituted 11-membered carbocycle. In some embodiments, R⁴¹ is optionally substituted 12-membered carbocycle.

In some embodiments, R⁴¹ is optionally substituted 3- to 12-membered heterocycle. In some embodiments, R⁴¹ is optionally substituted 3-membered heterocycle. In some embodiments, R⁴¹ is optionally substituted 4-membered heterocycle. In some embodiments, R⁴¹ is optionally substituted 5-membered heterocycle. In some embodiments, R⁴¹ is optionally substituted 6-membered heterocycle. In some embodiments, R⁴¹ is optionally substituted 7-membered heterocycle. In some embodiments, R⁴¹ is optionally substituted 8-membered heterocycle. In some embodiments, R⁴¹ is optionally substituted 9-membered heterocycle. In some embodiments, R⁴¹ is optionally substituted 10-membered heterocycle. In some embodiments, R⁴¹ is optionally substituted 11-membered heterocycle. In some embodiments, R⁴¹ is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms.

In some embodiments, R⁴¹ is selected from

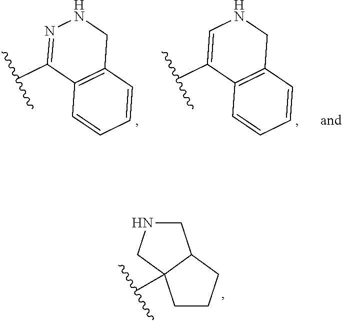

wherein each is optionally substituted with one or more substitutes. In some embodiments, R⁴¹ is optionally substituted

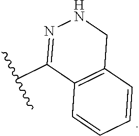 [SMILES: *C1=NNCc2ccccc21]

In some embodiments, R⁴¹ is optionally substituted

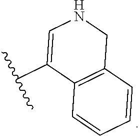 [SMILES: *C1=CNCc2ccccc21]

In some embodiments, R⁴¹ is optionally substituted

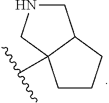 [SMILES: *C12CCCC1CNC2]

In some embodiments, the one or more substitutes are ═O. In some embodiments, the one or more substitutes are phenyl. In some embodiments, the one or more substitutes are —C(O)R⁴². In some embodiments, R⁴² is optionally substituted 3- to 12-membered heterocycle. In some embodiments, R⁴² is optionally substituted 3-membered heterocycle. In some embodiments, R⁴² is optionally substituted 4-membered heterocycle. In some embodiments, R⁴² is optionally substituted 5-membered heterocycle. In some embodiments, R⁴² is optionally substituted 6-membered heterocycle. In some embodiments, R⁴² is optionally substituted 7-membered heterocycle. In some embodiments, R⁴² is optionally substituted 8-membered heterocycle. In some embodiments, R⁴² is optionally substituted 9-membered heterocycle. In some embodiments, R⁴² is optionally substituted 10-membered heterocycle. In some embodiments, R⁴² is optionally substituted 11-membered heterocycle. In some embodiments, R⁴² is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, the heterocycle has two nitrogen atoms. In some embodiments, R⁴² is

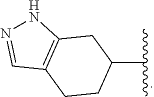 [SMILES: *C1CCc2cn[nH]c2C1]

In some embodiments, R⁴¹ is selected from

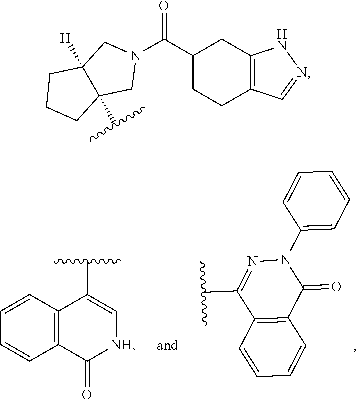 [SMILES: *[C@]12CCC[C@H]1CN(C(=O)C1CCc3cn[nH]c3C1)C2 ; *c1c[nH]c(=O)c2ccccc12 ; *c1nn(-c2ccccc2)c(=O)c2ccccc12]

In some embodiments, R⁴¹ is

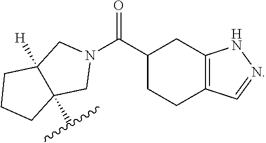 [SMILES: *[C@]12CCC[C@H]1CN(C(=O)C1CCc3cn[nH]c3C1)C2]

In some embodiments,
R⁴¹ is

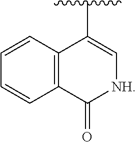 [SMILES: *c1c[nH]c(=O)c2ccccc12]

In some embodiments, R⁴¹ is

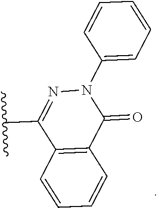 [SMILES: *c1nn(-c2ccccc2)c(=O)c2ccccc12]

In some embodiments, the compound having a structure of Formula (IV) is selected from the group consisting of:

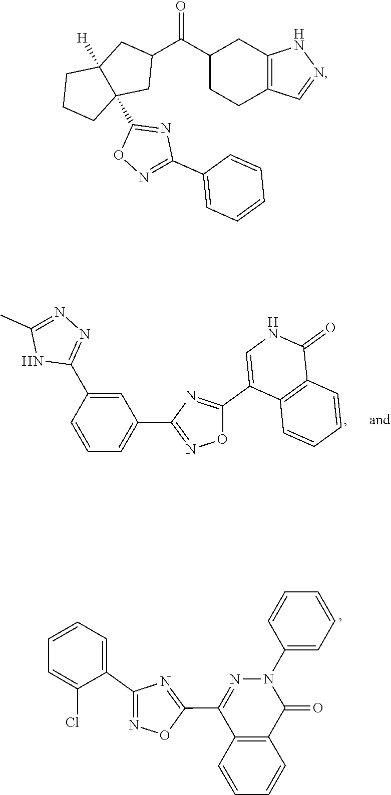 [SMILES: O=C(C1CCc2cn[nH]c2C1)C1C[C@@H]2CCC[C@]2(c2nc(-c3ccccc3)no2)C1 ; Cc1nnc(-c2cccc(-c3noc(-c4c[nH]c(=O)c5ccccc45)n3)c2)[nH]1 ; O=c1c2ccccc2c(-c2nc(-c3ccccc3Cl)no2)nn1-c1ccccc1]

and a salt thereof.

In certain aspects, the present disclosure provides a method for treating, preventing, or providing a relief for a disease, disorder, or condition in a subject in need thereof, comprising administering the subject a composition comprising a compound having a structure of Formula (V) or salt thereof,

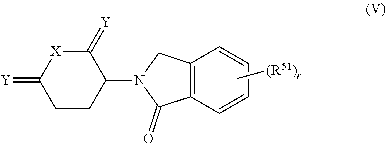

or a salt thereof,

wherein
X is selected from O, S, and NH;
Y is selected from O, S, and NH;
each R⁵¹ is independently selected at each occurrence from halogen, —OR⁵², —CN, —NO₂, —NR⁵²₂, C₁₋₁₀ alkyl, —C₁₋₁₀ haloalkyl, —OC(O)R⁵², —OCH₂C(O)R⁵², C₃₋₁₂ carbocycle, and optionally substituted 3- to 12-membered heterocycle;
   R⁵² is selected from hydrogen, C₁₋₆ alkyl, C₂₋₆ alkenyl, C₂₋₆ alkynyl, C₃₋₁₂ carbocycle, and 3- to 12-membered heterocycle, each of which is optionally substituted with one or more substituents selected from ═O, phenyl, —OH and optionally substituted 3- to 12-membered heterocycle; and
r is selected from 0, 1, 2, or 3.

In some embodiments, X is O. In some embodiments, X is S. In some embodiments, X is NH.

In some embodiments, Y is O. In some embodiments, Y is S. In some embodiments, Y is NH.

In some embodiments, each R⁵¹ is independently selected at each occurrence from —OR⁵², —OCH₂C(O)R⁵², and optionally substituted 3- to 12-membered heterocycle.

In some embodiments, R⁵¹ is —OR⁵². In some embodiments, R⁵² is hydrogen. In some embodiments, R⁵² is selected from —C₁₋₆ alkyl. In some embodiments, R⁵² is —C₁ alkyl. In some embodiments, R⁵² is —C₂ alkyl. In some embodiments, R⁵² is —C₃ alkyl. In some embodiments, R⁵² is —C₄ alkyl. In some embodiments, R⁵² is —C₅ alkyl. In some embodiments, R⁵² is —C₆ alkyl. In some embodiments, R⁵¹ is —OH. In some embodiments, R⁵¹ is —OCH₃. In some embodiments, R⁵¹ is —OCH₂CH₃. In some embodiments, R⁵¹ is —OCH₂CH₂CH₃.

In some embodiments, R⁵¹ is —OCH₂C(O)R⁵². In some embodiments, R⁵² is optionally substituted 3- to 12-membered heterocycle. In some embodiments, R⁵² is optionally substituted 3-membered heterocycle. In some embodiments, R⁵² is optionally substituted 4-membered heterocycle. In some embodiments, R⁵² is optionally substituted 5-membered heterocycle. In some embodiments, R⁵² is optionally substituted 6-membered heterocycle. In some embodiments, R⁵² is optionally substituted 7-membered heterocycle. In some embodiments, R⁵² is optionally substituted 8-membered heterocycle. In some embodiments, R⁵² is optionally substituted 9-membered heterocycle. In some embodiments, R⁵² is optionally substituted 10-membered heterocycle. In some embodiments, R⁵² is optionally substituted 11-membered heterocycle. In some embodiments, R⁵² is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom. In some embodiments, R⁵² is selected from

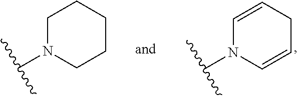 [SMILES: *N1CCCCC1 ; *N1C=CCC=C1]

optionally substituted with one or more substitutes.

In some embodiments, R⁵² is optionally substituted with one substituent selected from —OH and 3- to 12-membered heterocycle. In some embodiments, R⁵² is optionally substituted with —OH. In some embodiments, R⁵² is optionally substituted with 3-membered heterocycle. In some embodiments, R⁵² is optionally substituted with 4-membered heterocycle. In some embodiments, R⁵² is optionally substituted with 5-membered heterocycle. In some embodiments, R⁵² is optionally substituted with 6-membered heterocycle. In some embodiments, R⁵² is optionally substituted with 7-membered heterocycle. In some embodiments, R⁵² is optionally substituted with 8-membered heterocycle. In some embodiments, R⁵² is optionally substituted with 9-membered heterocycle. In some embodiments, R⁵² is optionally substituted with 10-membered heterocycle. In some embodiments, R⁵² is optionally substituted with 11-membered heterocycle. In some embodiments, R⁵² is optionally substituted with 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom.

In some embodiments, R⁵² is selected from

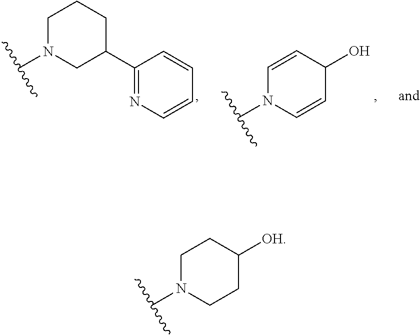 [SMILES: *N1CCCC(c2ccccn2)C1 ; *N1C=CC(O)C=C1 ; *N1CCC(O)CC1]

In some embodiments, R⁵² is

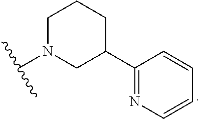 [SMILES: *N1CCCC(c2ccccn2)C1]

In some embodiments, R⁵² is

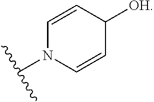 [SMILES: *N1C=CC(O)C=C1]

In some embodiments, R⁵² is

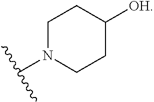 [SMILES: *N1CCC(O)CC1]

In some embodiments, R⁵¹ is selected from

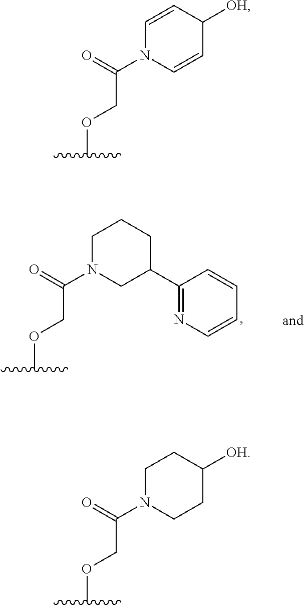 [SMILES: *OCC(=O)N1C=CC(O)C=C1 ; *OCC(=O)N1CCCC(c2ccccn2)C1 ; *OCC(=O)N1CCC(O)CC1]

In some embodiments, R⁵¹ is

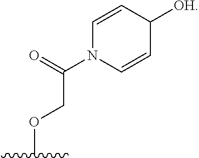 [SMILES: *OCC(=O)N1C=CC(O)C=C1]

In some embodiments, R⁵¹ is

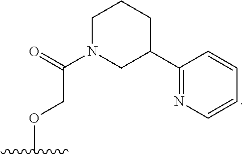 [SMILES: *OCC(=O)N1CCCC(c2ccccn2)C1]

In some embodiments, R⁵¹ is

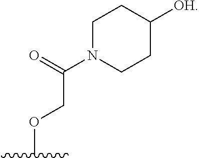 [SMILES: *OCC(=O)N1CCC(O)CC1]

In some embodiments, R⁵¹ is optionally substituted 3- to 12-membered heterocycle. In some embodiments, R⁵¹ is optionally substituted 3-membered heterocycle. In some embodiments, R⁵¹ is optionally substituted 6-membered heterocycle. In some embodiments, R⁵¹ is optionally substituted 4-membered heterocycle. In some embodiments, R⁵¹ is optionally substituted 5-membered heterocycle. In some embodiments, R⁵¹ is optionally substituted 6-membered heterocycle. In some embodiments, R⁵¹ is optionally substituted 7-membered heterocycle. In some embodiments, R⁵¹ is optionally substituted 8-membered heterocycle. In some embodiments, R⁵¹ is optionally substituted 9-membered heterocycle. In some embodiments, R⁵¹ is optionally substituted 10-membered heterocycle. In some embodiments, R⁵¹ is optionally substituted 11-membered heterocycle. In some embodiments, R⁵¹ is optionally substituted 12-membered heterocycle. In some embodiments, the heterocycle has at least one heteroatom selected from nitrogen, oxygen, and sulfur. In some embodiments, the heterocycle has a heteroatom selected from nitrogen, oxygen, and sulfur. In some embodiments, the heterocycle has at least one nitrogen atom. In some embodiments, the heterocycle has a nitrogen atom.

In some embodiments, R⁵¹ is selected from optionally substituted pyridine, tetrahydropyran, pyridazine, pyrimidine, and pyrazine. In some embodiments, R⁵¹ is selected from optionally substituted pyridine. In some embodiments, R⁵¹ is selected from optionally substituted tetrahydropyran. In some embodiments, R⁵¹ is selected from optionally substituted pyridazine. In some embodiments, R⁵¹ is selected from optionally substituted pyrimidine. In some embodiments, R⁵¹ is selected from optionally substituted pyrazine. In some embodiments, R⁵¹ is

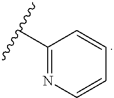 [SMILES: *c1ccccn1]

In some embodiments, r is 1 or 2. In some embodiments, r is 1. In some embodiments, r is 2.

In some embodiments, the compound is selected from the group consisting of:

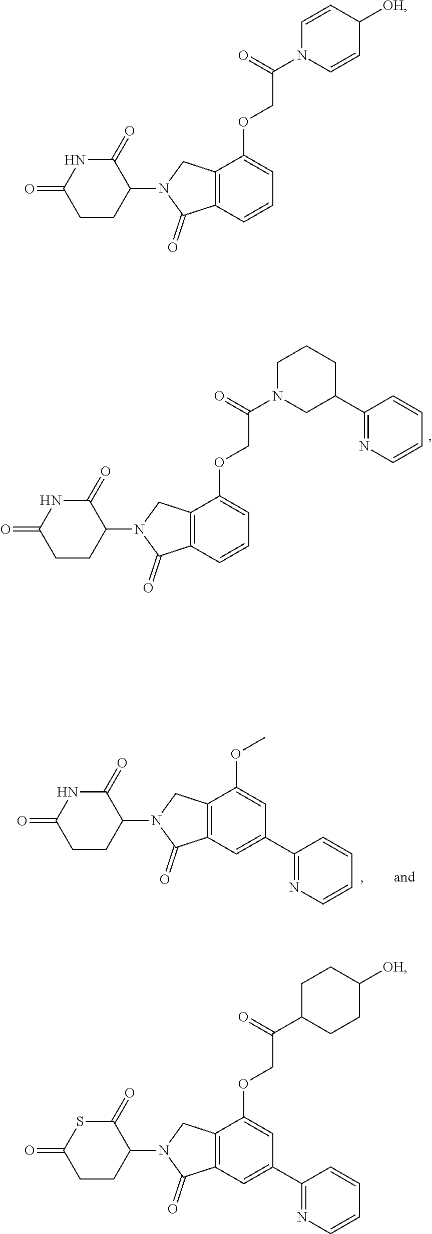 [SMILES: O=C1CCC(N2Cc3c(OCC(=O)N4C=CC(O)C=C4)cccc3C2=O)C(=O)N1 ; O=C1CCC(N2Cc3c(OCC(=O)N4CCCC(c5ccccn5)C4)cccc3C2=O)C(=O)N1 ; COc1cc(-c2ccccn2)cc2c1CN(C1CCC(=O)NC1=O)C2=O ; O=C1CCC(N2Cc3c(OCC(=O)C4CCC(O)CC4)cc(-c4ccccn4)cc3C2=O)C(=O)S1]

and a salt thereof.

In certain aspects, the present disclosure provides a method for treating, preventing, or providing a relief for a disease, disorder, or condition in a subject in need thereof, comprising administering the subject a composition comprising a compound having a structure selected from a group consisting of:

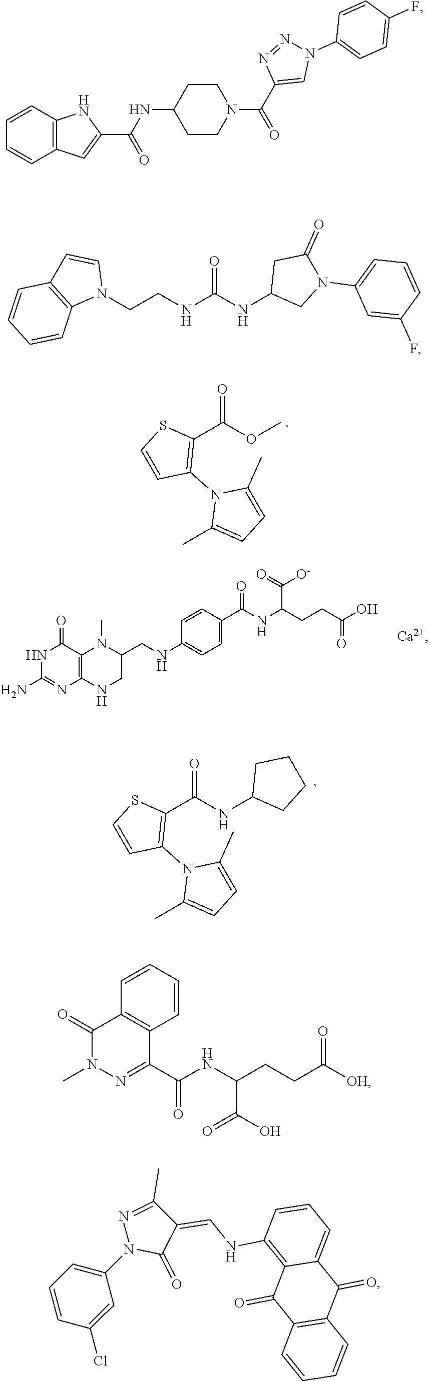

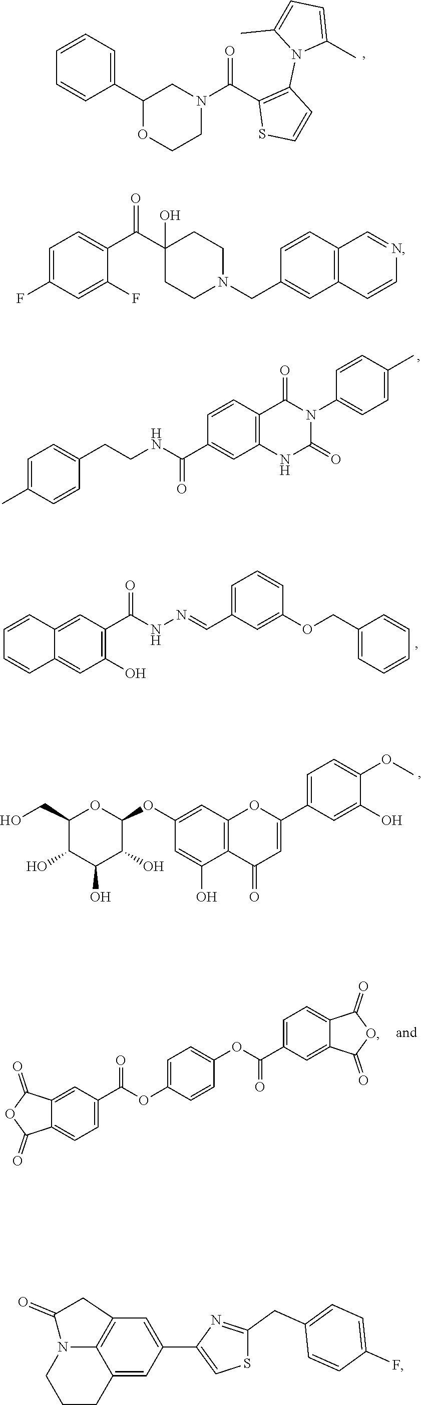

and a salt thereof.

In some embodiments, the compounds or a salt thereof disclosed herein may inhibit IL-4 receptor signaling. In some embodiments, the compounds or salt thereof in the present disclosure may directly bind to IL-4 receptor and inhibit its downstream action. In some embodiments, the compound disclosed herein may result in a functional decrease in the expression of IL-4 and/or IL-13 in activated Th2 cells. In some embodiments, the compounds or a salt thereof disclosed herein may reduce the expression of IL-4 and IL-13 was tested in primary human Th2 cells. In some embodiments, the compound or salt of the present disclosure may not alter the proliferation or viability of healthy human dermal fibroblasts. In some embodiments, the compound or salt of the present disclosure may activate monocyte-derived dendritic cells (moDCs).

In some embodiments, the compounds and a salt thereof disclosed herein are useful for treating or providing a relief for a disease, disorder, or condition which are improved, inhibited, or ameliorated by reducing IL-4 and/or IL-13 activity. In some embodiments, the compounds and a salt thereof disclosed herein may treat or provide a relief for a disease, disorder, or condition which are improved, inhibited, or ameliorated by reducing IL-4 and/or IL-13 activity. In some embodiments, the compounds and a salt thereof disclosed herein may treat a disease, disorder, or condition which are improved, inhibited, or ameliorated by reducing IL-4 and/or IL-13 activity. In some embodiments, the compounds and a salt thereof disclosed herein may provide a relief for a disease, disorder, or condition which are improved, inhibited, or ameliorated by reducing IL-4 and/or IL-13 activity. In some embodiments, the compounds and a salt thereof disclosed herein are useful for treating a disease, disorder, or condition which are improved, inhibited, or ameliorated by reducing IL-4 activity. In some embodiments, the compounds and a salt thereof disclosed herein may treat a disease, disorder, or condition which are improved, inhibited, or ameliorated by reducing IL-4 activity. In some embodiments, the compounds and a salt thereof disclosed herein are useful for treating a disease, disorder, or condition which are improved, inhibited, or ameliorated by reducing IL-13 activity. In some embodiments, the compounds and a salt thereof disclosed herein may treat a disease, disorder, or condition which are improved, inhibited, or ameliorated by reducing IL-13 activity. In some embodiments, the compounds and a salt thereof disclosed herein are useful for providing a relief for a disease, disorder, or condition which are improved, inhibited, or ameliorated by reducing IL-13 activity. In some embodiments, the compounds and a salt thereof disclosed herein are useful for providing a relief for a disease, disorder, or condition which are improved, inhibited, or ameliorated by reducing IL-13 activity. In some embodiments, the compounds and a salt thereof disclosed herein may provide a relief for a disease, disorder, or condition which are improved, inhibited, or ameliorated by reducing IL-13 activity. The disease, disorder, or condition comprises those characterized by abnormal or excess expression of IL-4 and/or IL-13, or by an abnormal host response to IL-4/IL-13 production. The disease, disorder, or condition comprises those characterized by abnormal or excess expression of IL-4. The disease, disorder, or condition comprises those characterized by abnormal or excess expression of IL-13. The disease, disorder, or condition comprises those characterized by an abnormal host response to IL-4 production. The disease, disorder, or condition comprises those characterized by an abnormal host response to IL-13 production. In some embodiments, the subject in need of an increased inflammatory response or in need of treatment according to the methods provided herein is a subject who has, is diagnosed as having, or is in need of treatment for asthma, allergies, cancer, atopic dermatitis, chronic rhinosinusitis with nasal polyposis (CRSwNP), eosinophilic esophagitis, an infection, or an autoimmune condition.

In some embodiments, the compounds and a salt thereof disclosed herein are useful for treating a disease, disorder, or condition of skin. In some embodiments, the compounds and a salt thereof disclosed herein may treat a disease, disorder, or condition of skin. In some embodiments, the condition of skin comprises redness, dryness, sensitization not related to an underlying disease or disorder. In some embodiments, the disease, disorder, or condition of skin comprises eczema. In some embodiments, the compounds and a salt thereof disclosed herein are useful for treating eczema. In some embodiments, the compounds and a salt thereof disclosed herein may treat eczema. In some embodiments, eczema comprises mild to moderate eczema.

The skin that may be treated by the methods provided herein include but are not limited to scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, general areas of skin that rub against each other, or a combination thereof.

In some embodiments, the compounds and a salt thereof disclosed herein are useful for treating a peripheral immune condition. In some embodiments, the compounds and a salt thereof disclosed herein may treat a peripheral immune condition. In some embodiments, the compounds and a salt thereof disclosed herein are useful for treating an inflammatory condition. In some embodiments, the compounds and a salt thereof disclosed herein may treat an inflammatory condition. In some embodiments, the peripheral immune/inflammatory condition includes but is not limited to atopic dermatitis, contact dermatitis, dyshidrotic eczema, neurodermatitis, nummular eczema, seborrheic dermatitis, stasis dermatitis, or a combination thereof. In some embodiments, the peripheral immune/inflammatory condition comprises vitiligo and alopecia. In some embodiments, the peripheral immune/inflammatory condition comprises atopic dermatitis. In some embodiments, the peripheral immune/inflammatory condition comprises contact dermatitis. In some embodiments, the peripheral immune/inflammatory condition comprises dyshidrotic eczema. In some embodiments, the peripheral immune/inflammatory condition comprises neurodermatitis. In some embodiments, the peripheral immune/inflammatory condition comprises nummular eczema. In some embodiments, the peripheral immune/inflammatory condition comprises seborrheic dermatitis. In some embodiments, the peripheral immune/inflammatory condition comprises stasis dermatitis. In some embodiments, the peripheral immune/inflammatory condition comprises vitiligo. In some embodiments, the peripheral immune/inflammatory condition comprises alopecia.

Beneficial or desired results of the administration of a composition comprising the compounds disclosed herein include, but are not limited to, a therapeutic benefit. In some embodiments, the administration of a composition comprising the compounds disclosed herein may reduce dryness, itching, redness, roughness, or flakiness of skin. In some embodiments, the administration may reduce roughness of skin. In some embodiments, the administration of a composition comprising the compounds disclosed herein may reduce flakiness of skin. In some embodiments, the administration of a composition comprising the compounds disclosed herein may reduce dryness of skin. In some embodiments, the administration reduces itching of skin. In some embodiments, the administration may reduce redness of skin. Beneficial or desired results of the administration of the compounds disclosed herein include, but are not limited to, a cosmetic benefit.

In some embodiments, the administration of a composition comprising the compounds or a salt thereof disclosed herein may decrease the expression of interleukin 4 (IL-4) and/or interleukin 13 (IL-13). In some embodiments, the administration of a composition comprising the compounds or a salt thereof disclosed herein may decrease the expression of interleukin 4 (IL-4). In some embodiments, the administration of a composition comprising the compounds or a salt thereof disclosed herein may decrease the expression of interleukin 13 (IL-13). In some embodiments, the administration of a composition comprising the compounds or a salt thereof disclosed herein may decrease the expression of IL-4 in activated Th2 cells. In some embodiments, the administration of a composition comprising the compounds or a salt thereof disclosed herein may decrease the expression of IL-13 in activated Th2 cells. In some embodiments, the administration of a composition comprising the compounds or a salt thereof disclosed herein may block or reduce engagement between IL-4 and interleukin 4 receptor, alpha (IL-4Rα). In some embodiments, the administration of a composition comprising the compounds or a salt thereof disclosed herein may block engagement between IL-4 and interleukin 4 receptor, alpha (IL-4Rα). In some embodiments, the administration of a composition comprising the compounds or a salt thereof disclosed herein may reduce engagement between IL-4 and interleukin 4 receptor, alpha (IL-4Rα). In some embodiments, the administration of the compounds or a salt thereof disclosed herein may block or reduce engagement between a receptor complex that is composed of interleukin 4 receptor, alpha (IL-4Rα), and an interacting cytokine (ie: IL-4, IL-13, etc.). In some embodiments, the administration of the compounds or a salt thereof disclosed herein may block engagement between a receptor complex that is composed of interleukin 4 receptor, alpha (IL-4Rα), and an interacting cytokine (ie: IL-4, IL-13, etc.). In some embodiments, the administration of a composition comprising the compounds or a salt thereof disclosed herein may reduce engagement between a receptor complex that is composed of interleukin 4 receptor, alpha (IL-4Rα), and an interacting cytokine (ie: IL-4, IL-13, etc.). In some embodiments, the administration of a composition comprising the compounds disclosed herein or a salt may not activate monocyte-derived dendritic cells (moDCs).

In some embodiments, the administration of a composition comprising the compounds or a salt thereof disclosed herein may decrease the expression of IL-4 in activated reporter HEK 293 cells. In some embodiments, the administration of a composition comprising the compounds or a salt thereof disclosed herein may decrease the expression of IL-13 in activated reporter HEK 293 cells. In some embodiments, the reporter HEK 293 cell comprises human IL-4/IL-13 SEAP reporter cells.

In some embodiments, the composition comprising a compound disclosed herein further comprises an additive selected from the group consisting of pharmaceutically acceptable carriers, excipients, adjuvants, diluents, and combinations thereof. The excipients include, but are not limited to, water, preservatives, antioxidants, sunscreen agents, vitamins, proteins, amino acids, natural extracts such as plant extracts, humectants, fragrances, perfumes, oils, emollients, lubricants, butters, penetrants, water softeners, salts, chelating agents, thickeners, viscosity modifiers, polymers, resins, film formers, emulsifiers, volatiles, liquid vehicles, carriers, pH adjusting agents, neutralizing agents, buffers, and combinations thereof.

The composition comprising a compound disclosed herein may be formulated as any suitable physical form. Suitable forms include, but are not limited to an aerosol, a liquid, a gel, a semisolid, a solid, or a powder. In some embodiments, the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol.

In some embodiments, the composition is formulated as an edible supplement, a beverage, or a jelly.

In some embodiments, the compounds as per the present invention may be administered to a subject via various different routes. Different routes include, but are not limited to a topical application, a direct injection, an inhalational route, a transdermal route, an intranasal route, an oral route, a sublingual route, a buccal route, an intravenous injection, an intramuscular injection, a subcutaneous injection, a rectal route, a vaginal route, and/or any combination of the above administration routes.

In some embodiments, the method disclosed herein may be used as a single or a combination therapies.

### Kits

This disclosure also provides a kit comprising the composition disclosed herein. In some embodiments, the kit further comprises an applicator. In some embodiments, the applicator includes but is not limited to, a brush, a comb, a cotton swab, a spatula, a spoon, a dropper, a spray nozzle, a squeeze bottle, a tottle, or a combination thereof. In some embodiments, the kit further comprises written instructions for the method disclosed herein.

### EXAMPLES

The invention now being generally described, it will be more readily understood by reference to the following examples which are included merely for purposes of illustration of certain aspects and embodiments of the present invention, and are not intended to limit the invention in any way.

### Example 1: Lead Selection

440 chemicals were filtered down to 27 chemicals by thresholding the expression level of IL-4 and IL-13 among differentiated CD4+ Th2 cells, in combination with a battery of specificity and cellular toxicity studies. The 27 chemicals were further subject to repeat screening.

### Example 2: Experimental Details

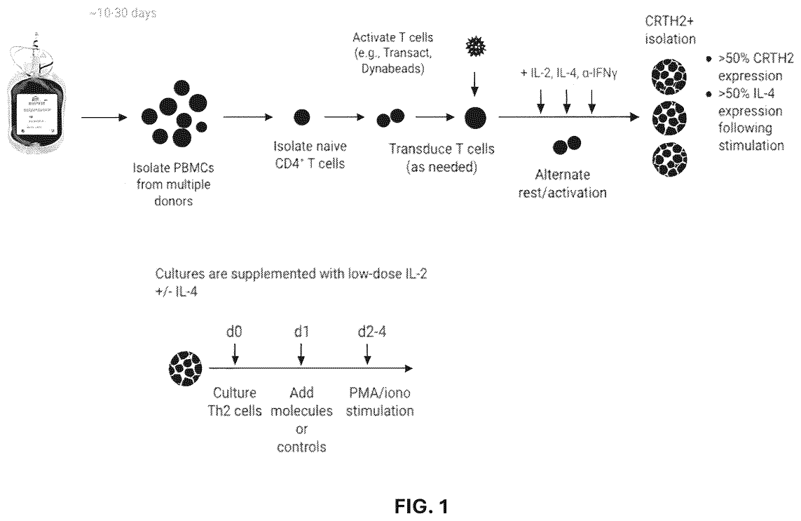

To produce T helper 2 (Th2) cells, peripheral blood mononuclear cells (PBMCs) were harvested from healthy donor leukopaks using Ficoll gradients. Naive CD4+ T cells were further isolated, then activated using TransAct or Dynabeads (depending on the donor). CD4+ T cells were activated for two days in the presence of low dose IL-2 (30 IU/mL) in X-VIVO-15 media containing 5% human AB serum (activation cycle). On the second day, the media was exchanged and supplemented with interleukin 4 (IL-4) (20 ng/mL), anti-IFN-gamma antibody (1 μg/mL), and interleukin 2 (IL-2) (rest cycle). The cells were cultured for an additional five days then evaluated for Prostaglandin D2 receptor 2 (DP2 or CRTH2) expression and IL-4 and interleukin 13 (IL-13) expression following phorbol myristate acetate (PMA)/ionomycin stimulation. If the expression of CRTH2 was >50%, the cells were frozen and used in Th2 IL-4/IL-13 bioactivity assays. If the expression of CRTH2 <50%, then the cells were restimulated using TransAct (or Dynabeads) and maintained on similar cycles of activation (2 days) and rest (5 days), until the expression of CRTH2 exceeded 50%. Typically, no more than 4 cycles are needed before the Th2 cells may be harvested and frozen. FIG. 1 shows the experimental details.

For testing the ability of a compound or salt of the present disclosure to reduce the expression of IL-4 and IL-13 was tested in primary human Th2 cells, the abovementioned cells were thawed, then cultured for one day in IL-2 containing media. The Th2 cells were treated with compounds added at varying concentrations for three days, after which, the cells were washed, and stimulated with PMA/ionomycin for 6 hrs prior to readabout via intracellular flow cytometry.

### Example 3: Assay of Functional Th2-Blocking Activity Using Primary Human T Helper 2 (Th2) Cells

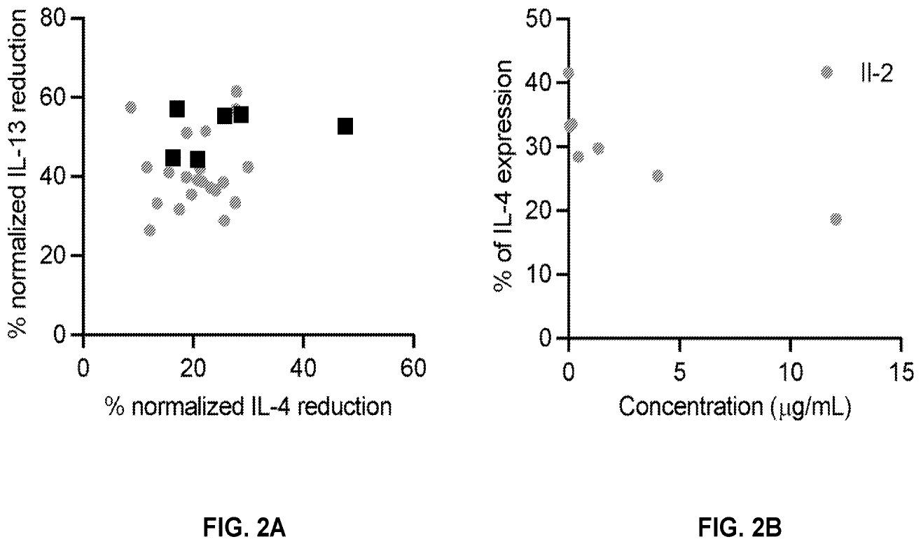

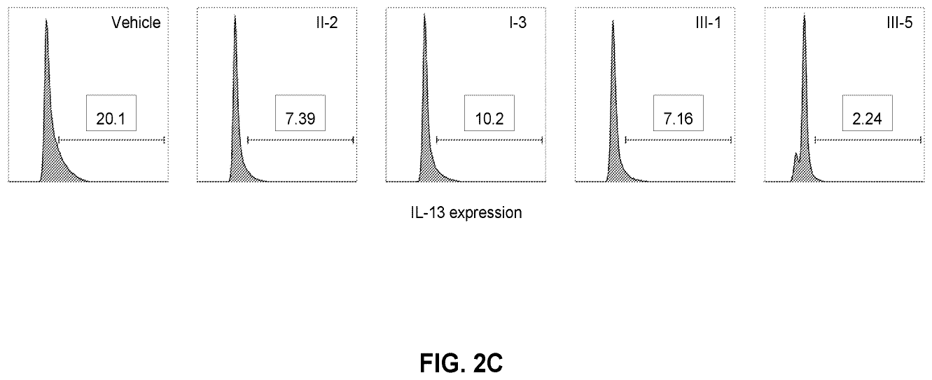

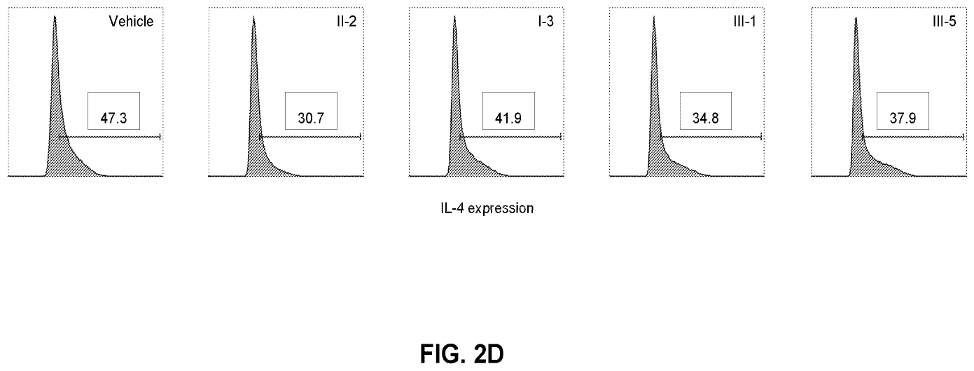

The ability of a compound or salt of the present disclosure to reduce the expression of IL-4 and IL-13 was tested in primary human Th2 cells. Naive, primary human CD4+ T cells were isolated from healthy donors and differentiated into Th2 cells using a combination of IL-4 cytokine and anti-IFN-gamma antibody, based on a modified protocol from Scott et al (Activation of Th2 cells downregulates CRTh2 through an NFAT1 mediated mechanism. PLoS One. 2018 Jul. 3; 13(7):e0199156). Th2 cells were CRTH2+ and expressed IL-4 and IL-13 following PMA/ionomycin stimulation. Prior to PMA/ionomycin stimulation, Th2 cells were co-cultured with a compound or salt of the present disclosure for 3 days and then stimulated for 6 hours. Intracellular flow cytometry was performed to investigate the expression of IL-4 and IL-13 among stimulated CD4+ Th2 cells. The compound or salt was evaluated across a 2-log concentration range. Th2 cells were cultured in X-VIVO-15 media with human AB serum and supplemented with low dose IL-2 (30 IU/mL). The antibody panel that was used is listed below. All antibodies were obtained from Biolegend. The results are presented in FIG. 2A-FIG. 2D.

TABLE 7

| Fluorophore | Stain | Clone |
|---|---|---|
| UV450 | Live/Dead stain | N/A |
| PerCP/Cy5.5 | CD3 | HIT3a |
| BV510 | CD4 | SK3 |
| APC-Fire750 | CD8 | SK1 |
| BV421 | IL-13 | JES10-5A2 |
| BV711 | CD25 | M-A251 |
| PE/Cy7 | IL-2 | MQ1-17H12 |
| APC | IFNg | 4S.B3 |
| FITC | IL-4 | MP4-25D2 |

### Example 4: Specificity Assay of Human Dermal Fibroblast Cells (hDFC) Proliferation

The ability of a compound or salt of the present disclosure to not alter the proliferation or viability of healthy human dermal fibroblasts was tested in a cell viability assay, such as the Promega CellTiter-Glo® Luminescent Cell Viability Assay, MTT cell proliferation assay (ATCC® 30-1010K) or manual cell counting. The compound or salt was added at two concentrations spanning a 1-log range. Fibroblasts were cultured on tissue culture-treated plastic using predefined growth media (i.e., fibroblast growth media; Promocell) containing basic fibroblast growth factor (FGFβ).

### Example 5: Specificity Assay on Human Dermal Microvascular Endothelial Cells (HDMEC)

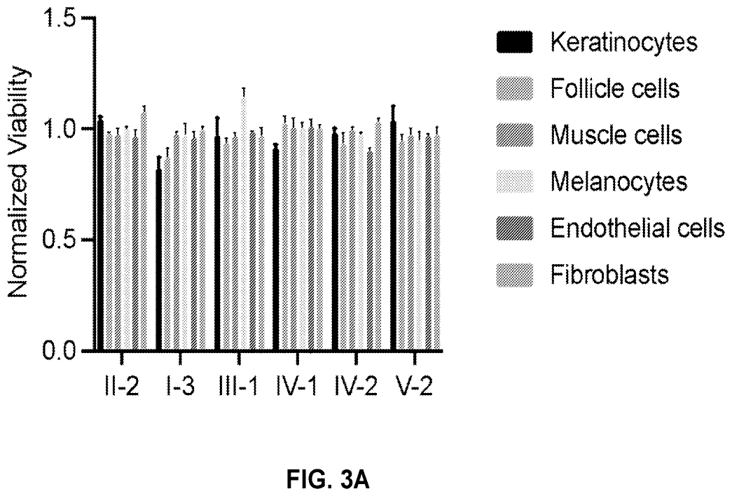

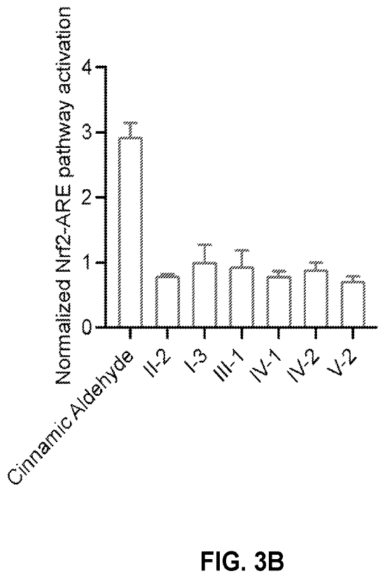

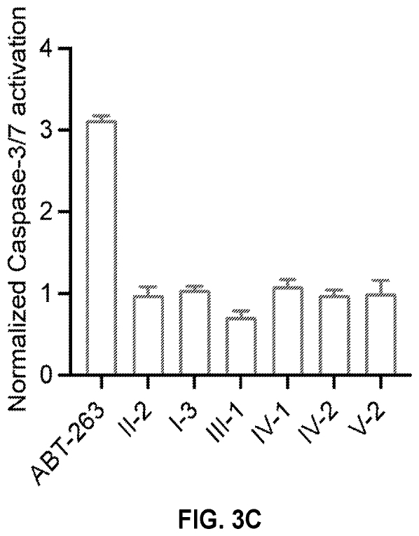

The ability of a compound or salt of the present disclosure to impact viability of of human adult dermal microvascular endothelial cells was tested using a cell viability assay, such as Promega CellTiter-Glo® Luminescent Cell Viability Assay, MTT cell proliferation assay (ATCC® 30-1010K) or cell counting after 48 hr. The compound or salt was added at two concentrations spanning a 1-log range. The results are presented in FIG. 3A-FIG. 3C.

### Example 6: Specificity Assay on Keratinocytes

Human adult epidermal keratinocytes were cultured on poly-L-lysine treated plates and the ability of a compound or salt of the present disclosure to impact proliferation was measured using a cell viability assay, such as Promega CellTiter-Glo® Luminescent Cell Viability Assay, MTT cell proliferation assay (ATCC® 30-1010K) or cell counting after 48 hr. The compound or salt was added at two concentrations spanning a 1-log range.

### Example 7: Specificity Assay on Primary Epidermal Melanocytes; Human Epidermal Melanocytes, Adult (HEMa)

The ability of a compound or salt of the present disclosure to impact viability of human adult epidermal melanocytes was tested using a cell viability assay, such as Promega CellTiter-Glo® Luminescent Cell Viability Assay, MTT cell proliferation assay (ATCC® 30-1010K) or cell counting after 48 hr. The compound or salt was added at least two concentrations spanning a 1-log range. Melanocytes derived from at least two different donors of varying degrees of basal skin pigmentation were used.

### Example 8: Calculations

Description of Calculation for: (1) Expression of Cytokine following Stimulation (% X Positive among CD4+ T cells), and (2) % Normalized Reduction of Cytokine Expression From Baseline in CD4+ T Cells

The expression of IL-4 and IL-13 was measured based on positive gated fractions among CD4+ Th2 cells. The number is expressed as a positive percentage, determined based on the fluorescence-minus-one (FMO) control. The max percent is 100%. The normalized reduction value uses the vehicle control (water) to normalize against using the following percent change formula. (Cmpd: compound)

$$\% Normalized Reduction = ( \frac{Cmpd \% positive alue - Vehicle \% positive value}{Vehicle \% positive value} ) \cdot 100 \%$$

TABLE 8
Expression of cytokine following stimulation (% X Positive
among CD4+ T cells) and % normalized reduction
of cytokine expression from baseline in CD4+ T Cells

| | Expression of Cytokine | | % Normalized Reduction of | |
|---|---|---|---|---|
| | following Stimulation (% X | | Cytokine Expression From | |
| | Positive among CD4+ T cells) | | Baseline in CD4+ T Cells | |
| ID# | IL-4 | IL-13 | IL-4 | IL-13 |
| II-2 | B | B | B | D |
| I-3 | B | C | B | C |
| III-1 | A | B | D | D |
| IV-1 | B | C | C | C |
| IV-2 | B | B | D | D |
| V-2 | A | B | D | D |
| IV-3 | B | C | C | B |
| VI-1 | B | C | D | B |
| VI-2 | B | D | D | A |
| III-2 | B | C | C | C |
| VI-3 | B | C | B | C |
| VI-4 | B | C | B | B |
| III-3 | B | B | B | D |
| VI-5 | B | C | B | B |
| VI-6 | C | C | A | B |
| III-4 | B | C | C | B |
| III-6 | B | C | B | B |
| VI-7 | B | C | D | B |
| III-7 | A | B | D | D |
| VI-8 | C | C | A | C |
| VI-9 | A | C | D | C |
| VI-10 | B | C | C | B |
| VI-11 | D | D | A | A |
| III-5 | C | B | A | D |
| VI-12 | A | A | D | D |
| VI-13 | B | C | C | B |
| VI-14 | B | B | C | D |
| Vehicle | | | | |
| (water) | | | | |

For the expression of cytokine following stimulation (% X Positive among CD4+ T cells) for IL-4, A represents 0% to less than 30%, B represents 30% to less than 35%, C represents 35% to less than 40%, and D represents 40% or greater. For the expression of cytokine following stimulation (% X Positive among CD4+ T cells) for IL-13, A represents 0% to less than 10%, B represents 10% to less than 14%, C represents 14% to less than 18%, and D represents 18% or greater.

For % normalized reduction of cytokine expression from baseline in CD4+ T cells for IL-14, A represents 0% to less than 15%, B represents 15% to less than 20%, C represents 20% to less than 25%, and D represents 25% or greater. For % normalized reduction of cytokine expression from baseline in CD4+ T cells for IL-13, A represents 0% to less than 30%, B represents 30% to less than 40%, C represents 40% to less than 50%, and D represents 50% or greater.

### Example 9: Toxicology/Safety

### In Silico Safety/Toxicology Studies

The in silico safety/toxicology profiles of compounds were determined by screening chemical structures against a series of computational models. These include the Pred-hERG 4.2 cardiotoxicity model as well as reproductive and developmental toxicity, carcinogenicity (genotoxic and non-genotoxic), skin sensitization, DNA mutation, and chromosomal aberration models from QSAR Toolbox.

### In Vitro Safety/Toxicology Studies

The safety/toxicology profiles of a compound or salt of the present disclosure was evaluated via a series of supplemental in vitro genotoxicity assays, including the SOS-chromotest to determine bacterial genotoxicity and the TK.6 micronucleus assay to determine chromosomal damage and forward mutations. Additionally, the KeratinoSens reporter assay was used to determine any potential sensitivity via the activation of a cytoprotective pathway.

### Standard Reactive Oxygen Species (ROS) Assays

Standard reactive oxygen species (ROS) assays are conducted to determine general cellular toxicity. Moreover, a series of sensitization tests are conducted to predict any potential skin sensitivity. These include the direct peptide reactivity assay to evaluate haptenization and an immunological assay using human immature monocyte-derived dendritic cells to evaluate any potential immunogenicity. These studies include mechanism-oriented studies, including metabolism and transport studies focusing on the ability of a compound or salt of the present disclosure to induce or inhibit cytochrome P450, passive/active transport using the Caco-2 cell line, a well-established compound transport assay. Finally, a repeated insult patch test is performed using a compound or salt of the present disclosure on 200 human subjects to predict any potential induced allergic contact dermatitis and associated responses.

### Example 10: Validation of IL-4 Receptor Binding Studies

### Cell-Based Assay

HEK-Blue IL-4/IL-13 cells are used to evaluate IL-4/IL-4R-alpha binding. HEK-Blue IL-4/IL-13 have been engineered to produce secreted alkaline phosphatase (SEAP) when exposed to IL-4 or IL-13 and have been previously used to identify IL-4 and IL-4R inhibitors. Compounds are tested with these cells at various dosages to evaluate dose dependent changes in IL-4/IL-4R-alpha binding inhibition. Full EC50 curves are generated for Tier1 compounds.

### Surface Plasmon Resonance-Based Assay

The ability of a compound or salt of the present disclosure to bind to IL-4 receptor is evaluated in a series of studies using surface plasmon resonance-based immunoassays to determine the biophysical interactions between representative compounds and IL-4 receptor.

These studies validate the ability of a compound or salt of the present disclosure to directly bind to IL-4 receptor and inhibit its downstream action.

### Example 11: Mechanistic Analysis

Using transcriptomics dataset, a series of mechanistic computational analyses are performed to identify possible mechanisms of action. These may include DE analyses followed by GO term and pathway enrichment to characterize the biological imprint of our lead chemicals. In addition, pseudo-time and cell-cycle analyses are performed to probe the developmental effects of these chemicals on target cells. The extracted insight in further investigations is validated using various models.

### Example 12: Biological Assay

### Interleukin-4 (IL-4) Signaling Inhibition

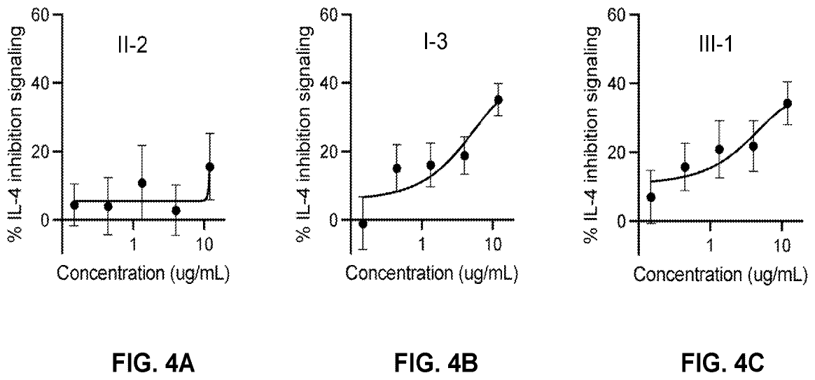

The compounds disclosed herein have demonstrated substantial efficacy in inhibiting IL-4 receptor signaling when tested under controlled conditions, as shown in FIG. 4A-FIG. 4C. In the experiment, engineered HEK-Blue™ IL-4/IL-13 cells were stimulated with a dose of 1.25 ng/mL of IL-4 and then subjected to co-culture with varying concentrations of the compounds for a period of 24 hours. The exception being compound II-2, which resulted in suppressed Th2 activity, other compounds (I-3 and III-1) manifested prominent IL-4 signaling inhibition. The provided data represent the mean±standard deviation of six technical replicates carried out across two experimental replicates.

### Blood Cell Toxicity Assay

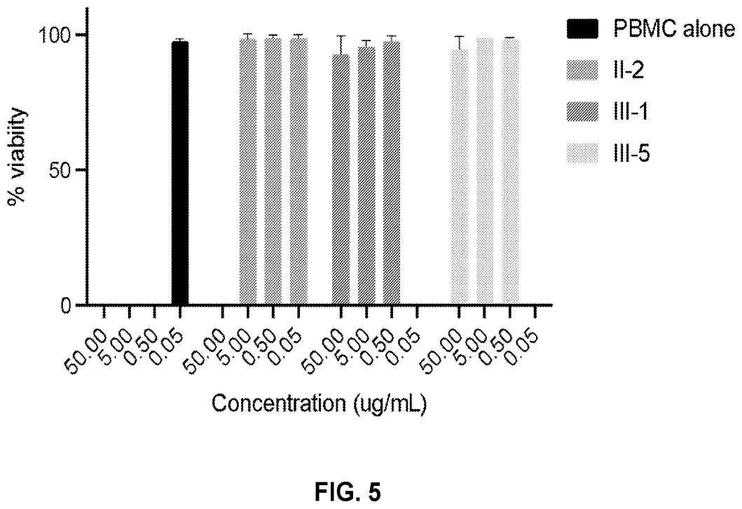

The compounds disclosed herein were characterized by their non-toxic effects on blood cells (FIG. 5). For the evaluation, peripheral blood mononuclear cells, procured from three different de-identified donors and stored in a frozen state, were revived and co-cultured with various quantities of the compounds of this disclosure. The high cell viability was observed at all applied concentrations, indicating the lack of blood cell toxicity associated with these compounds. The provided data represent the mean±standard deviation pertaining to the three individual donors.

Evaluation of Monocyte-Derived Dendritic Cell (moDCs) Activation

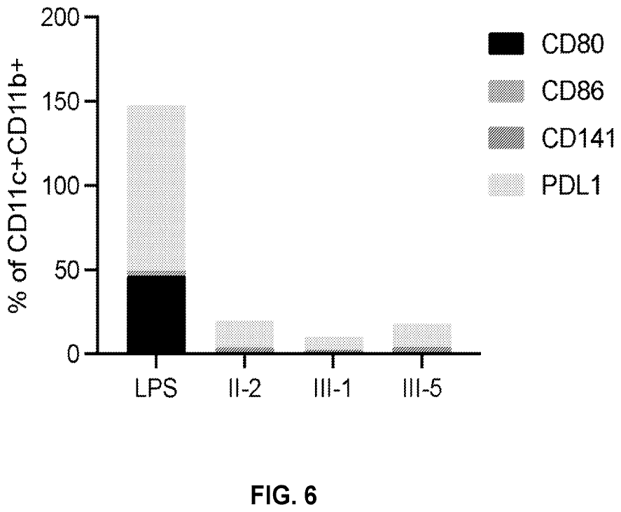

The compounds disclosed herein have demonstrated their inability to activate monocyte-derived dendritic cells (moDCs). The results are shown in FIG. 6. The experimental setup involved isolating CD14+ cells from human donor peripheral mononuclear cells and further differentiating these cells into immature moDCs by employing granulocyte-macrophage colony-stimulating factor (GM-CSF) and interleukin-4 (IL-4). The resultant moDCs were then exposed to the compounds at a concentration of 10 μg/mL. Lipopolysaccharide (LPS), derived from heat-killed E. coli, was included as a positive control to ascertain activation. In contrast to LPS, the compounds from this disclosure did not display any substantial activation of moDCs. The data presented signifies the mean of two technical replicates and is representative of two independent experimental procedures.

### Genotoxic Assay

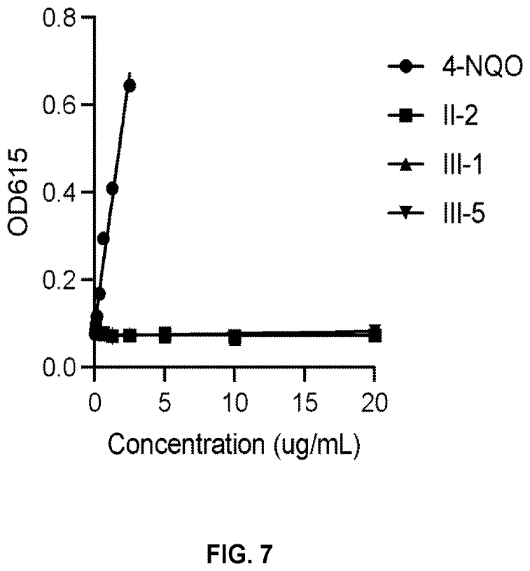

The compounds disclosed herein were characterized by their non-genotoxic nature. To assess the genotoxic potential of the compounds, the SOS chromotest was utilized. This chromotest is an internationally recognized, bacteria-based assay developed specifically for evaluating genotoxicity. In the experiments, 4-nitroquinoline 1-oxide (4-NQO) was used as a reference. As shown in FIG. 7, the result of the SOS chromotest confirmed that the compounds do not possess genotoxic properties. The presented data is representative of two independent experimental procedures.

### Evaluation of Inducing Oxidative Stress

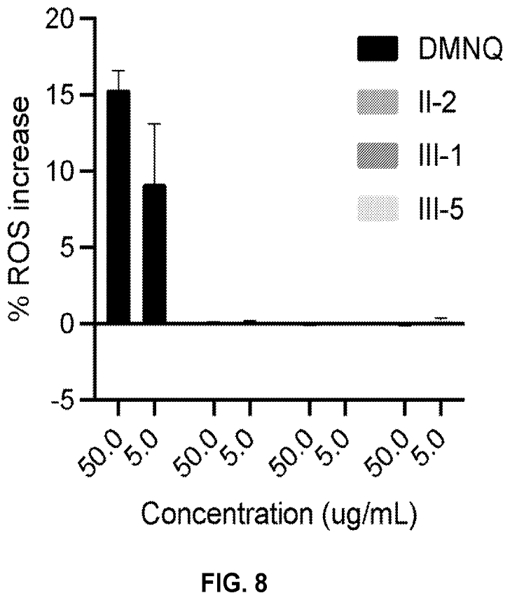

The compounds disclosed herein did not induce oxidative stress in primary human dermal papilla cells, as demonstrated in FIG. 8. The generation of intracellular reactive oxygen species (ROS), a key indicator of oxidative stress, by the compounds was evaluated using the ROS-Glo™ assay (Promega) at concentrations of either 50 μg/mL or 5 μg/mL. In comparison to 2,3-dimethoxy-1,4-naphthalenedione (DMNQ), a known carcinogen and inducer of oxidative stress, the compounds of this disclosure did not elevate the intracellular ROS levels. The presented data signifies the mean±standard deviation for three technical replicates.

### Example 13: Clinical Tests

### In Vitro Irritation Testing

The compounds disclosed herein were subjected to the HET-CAM (hen's egg-chorioallantoic membrane) test with a total of four replicates. This test was performed to assess the potential for membrane irritation, specifically ocular irritation, by applying a hydroalcoholic solution at a 0.15% concentration. The irritant potential is scored on a scale ranging from 0 to 21, which subsequently classifies the product into various categories: Non-irritant (0.0-0.9), Slight Irritant (1.0-4.9), Moderate Irritant (5.0-9.9), or Severe Irritant (10.0-21.0). The compounds disclosed herein were tested using this methodology, and all of them returned scores in the range of 3.50-5.00. This indicates that the compounds fall into the category of either slight irritants or practically non-irritants, confirming their minimal potential for causing irritation.

### Clinical Safety Testing

The compounds disclosed herein were rigorously tested for their dermal impact. A repeated insult patch test was conducted with 56 subjects to determine the potential for the compounds to cause dermal irritation or induce sensitization. In these experiments, the compounds were applied at a concentration of 0.15% by weight. None of the tested compounds demonstrated any potential to elicit dermal irritation or induce sensitization among the subjects.

### Clinical Efficacy Testing

A planned clinical study will utilize a cream formulation incorporating at least one compound or salt as disclosed herein. The study, devised as a three-arm trial involving a total of 90 subjects, will span over a period of one month. The trial will incorporate expert grading of skin conditions, digital photography, and subject self-assessments to systematically track both short-term and long-term improvements in skin condition. Two arms of the study will incorporate benchmark formulations, namely, a commercially available 1% hydrocortisone product and a 1% colloidal oatmeal vehicle control. The third arm will consist of a novel formulation integrating 1% colloidal oatmeal with at least one compound of the current disclosure. The endpoints of the study will include SCORAD (Scoring Atopic Dermatitis), EASI (Eczema Area and Severity Index) scores, and measurements of erythema at selected skin condition sites, as assessed by independent, expert clinical graders.

### Example 14: Compositions

The composition may be a cosmetic composition or a pharmaceutical composition. The composition comprises a cosmetically acceptable salt or a pharmaceutically acceptable salt. The composition may comprise one or more compounds disclosed herein. In some embodiments, the composition comprises one compound disclosed herein.

In some embodiments, the composition comprises a cosmetic composition or a pharmaceutical composition. In some embodiments, the composition comprises a cosmetically acceptable salt or a pharmaceutically acceptable salt. In some embodiments, the composition comprises one or more compounds disclosed herein.

TABLE 9 to TABLE 12 outline example compositions of a topical cream designed for a direct application to the affected skin area.

TABLE 9
Example of Composition

| Ingredient | Concentration (wt. %) |
|---|---|
| Water/Aqua/Eau | 50.00 |
| Trisodium Ethylenediamine | 0.20 |
| Disuccinate | |
| Glycerin | 5.00 |
| Propanediol | 2.00 |
| Hydroxyethyl Acrylate/Sodium | 0.50 |
| Acryloyldimethyl Taurate Copolymer | |
| Sodium Acryloyldimethyltaurate/VP | 0.75 |
| Crosspolymer | |
| Avena Sativa (Oat) Kernel Flour | 1.00 |
| Sodium Citrate | 0.15 |
| Citric Acid | 0.70 |
| Cetyl Alcohol | 1.00 |
| Coco-Caprylate/Caprate | 10.00 |
| Caprylic/Capric Triglyceride | 7.00 |
| Butyrospermum Parkii (Shea) Butter | 3.00 |
| Glyceryl Stearate (and) Polyethylene | 5.50 |
| glycol (PEG)-100 Stearate | |
| Dimethicone | 1.50 |
| Dimethyl Isosorbide | 7.50 |
| Ethoxydiglycol | 2.00 |
| Polyglyceryl-4 Caprate | 0.40 |
| First compound disclosed herein | 0.1 |
| Second compound disclosed herein | 0.05 |
| Tocopherol | 1.10 |
| Pentylene Glycol (and) Caprylyl Glycol | 0.05 |
| (and) Propanediol (and) Ethylhexylglycerin | |

TABLE 10
Example of Composition

| Ingredient | Concentration (wt. %) |
|---|---|
| Water/Aqua/Eau | 57.65 |
| Trisodium Ethylenediamine Disuccinate | 0.20 |
| Glycerin | 4.00 |
| Propanediol | 3.00 |
| Hydroxyethyl Acrylate/Sodium | 1.00 |
| Acryloyldimethyl Taurate Copolymer | |
| Sodium Acryloyldimethyltaurate/VP | 0.70 |
| Crosspolymer | |
| Avena Sativa (Oat) Kernel Flour | 1.00 |
| Sodium Citrate | 0.1 |
| Citric Acid | 0.20 |
| Cetyl Alcohol | 5.00 |
| Coco-Caprylate/Caprate | 1.00 |
| Caprylic/Capric Triglyceride | 5.00 |
| Butyrospermum Parkii (Shea) Butter | 1.00 |
| Glyceryl Stearate (and) PEG-100 Stearate | 7.00 |
| Dimethicone | 1.50 |
| Dimethyl Isosorbide | 8.50 |
| Ethoxydiglycol | 1.00 |
| Polyglyceryl-4 Caprate | 0.50 |
| Compound of the present disclosure | 0.1 |
| Tocopherol | 0.05 |
| Pentylene Glycol (and) Caprylyl Glycol | 1.50 |
| (and) Propanediol (and) Ethylhexylglycerin | |
| Water/Aqua/Eau | 57.65 |

TABLE 11
Example of Composition

| Ingredient | Concentration (wt. %) |
|---|---|
| Water/Aqua/Eau | 57.60 |
| Trisodium Ethylenediamine Disuccinate | 0.20 |
| Glycerin | 5.00 |
| Propanediol | 2.00 |
| Hydroxyethyl Acrylate/Sodium | 1.00 |
| Acryloyldimethyl Taurate Copolymer | |
| Sodium Acryloyldimethyltaurate/VP | 0.75 |
| Crosspolymer | |
| Avena Sativa (Oat) Kernel Flour | 1.00 |
| Sodium Citrate | 0.15 |
| Citric Acid | 0.10 |
| Cetyl Alcohol | 1.00 |
| Coco-Caprylate/Caprate | 5.00 |
| Caprylic/Capric Triglyceride | 5.00 |
| Butyrospermum Parkii (Shea) Butter | 3.00 |
| Glyceryl Stearate (and) PEG-100 Stearate | 5.00 |
| Dimethicone | 1.50 |
| Dimethyl Isosorbide | 7.50 |
| Ethoxydiglycol | 2.00 |
| Polyglyceryl-4 Caprate | 0.50 |
| Compound II-2 (5-oxo-N-[3-({3- | 0.05 |
| oxo-2H,3H-[1,2,4]triazolo[4, | |
| 3-a]pyridin-2-yl}methyl)phenyl]- | |
| 1H,5H-pyrazolo[1,5- | |
| a]quinazoline-3-carboxamide) | |
| Compound III-1 (4-(benzyloxy)-N-{3- | 0.05 |
| [(3,4-dihydro-2H-pyrrol-5- | |
| yl)sulfamoyl]phenyl}benzamide) | |
| Tocopherol | 0.10 |
| Pentylene Glycol (and) Caprylyl Glycol | 1.50 |
| (and) Propanediol (and) Ethylhexylglycerin | |

TABLE 12
Example of Composition

| Ingredient | Concentration (wt. %) |
|---|---|
| Water/Aqua/Eau | 57.65 |
| Trisodium Ethylenediamine Disuccinate | 0.20 |
| Glycerin | 5.00 |
| Propanediol | 2.00 |
| Hydroxyethyl Acrylate/Sodium | 1.00 |
| Acryloyldimethyl Taurate Copolymer | |
| Sodium Acryloyldimethyltaurate/VP | 0.75 |
| Crosspolymer | |
| Avena Sativa (Oat) Kernel Flour | 1.00 |
| Sodium Citrate | 0.15 |
| Citric Acid | 0.10 |
| Cetyl Alcohol | 1.00 |
| Coco-Caprylate/Caprate | 5.00 |
| Caprylic/Capric Triglyceride | 5.00 |
| Butyrospermum Parkii (Shea) Butter | 3.00 |
| Glyceryl Stearate (and) PEG-100 Stearate | 5.00 |
| Dimethicone | 1.50 |
| Dimethyl Isosorbide | 7.50 |
| Ethoxydiglycol | 2.00 |
| Polyglyceryl-4 Caprate | 0.50 |
| Compound of the present disclosure | 0.05 |
| Tocopherol | 0.10 |
| Pentylene Glycol (and) Caprylyl Glycol | 1.50 |
| (and) Propanediol (and) Ethylhexylglycerin | |
| Water/Aqua/Eau | 57.65 |

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method for treating, preventing, or providing a relief for a disease, disorder, or condition in a subject in need thereof, comprising administering the subject a composition comprising a compound having a structure of Formula (III) or salt thereof, 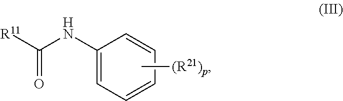
wherein
R¹¹ is selected from —CH₂R¹², —OR¹², —SR¹², —C(O)R¹², —C(O)OR¹², —OC(O)R¹², —OC(O)N(R¹²)₂, —NR¹²S(O)₂R¹², —C(O)N(R¹²)₂, —S(O)₂(R¹²), —S(O)₂N(R¹²)₂, optionally substituted C₃-C₁₂ carbocycle, and optionally substituted 5- to 14-membered heterocycle;
each R¹² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle;
each R²¹ is independently selected at each occurrence from —OH, halogen, C₁₋₆ alkyl, C₁₋₆ haloalkyl, OR²², —SR²², —C(O)R²², —C(O)OR²², —OC(O)R²², —OCH₂C(O)R²², —OC(O)N(R²²)₂, —OCH₂C(O)NHR²², —OCH₂C(O)N(R²²)₂, —NR²²S(O)₂R²², —C(O)N(R²²)₂, —S(O)₂(R²²), —S(O)₂NHR²², —S(O)₂N(R²²)₂, —NHC(O)R²², optionally substituted C₃-C₁₂ carbocycle, and optionally substituted 5- to 14-membered heterocycle;
each R²² is independently selected at each occurrence from hydrogen, C₁₋₆ alkyl, C₂₋₆ alkenyl, C₂₋₆ alkynyl, optionally substituted C₃-C₁₂ carbocycle, and optionally substituted 5- to 14-membered heterocycle; and
p is selected from 0, 1, 2, 3, or 4.

2. The method of claim 1, wherein R¹¹ is —CH₂R¹², wherein R¹² is optionally substituted C₃-C₁₂ carbocycle or optionally substituted 5- to 14-membered heterocycle.

3. (canceled)

4. (canceled)

5. (canceled)

6. The method of claim 1, wherein R¹¹ is optionally substituted C₃-C₁₂ carbocycle or optionally substituted 5- to 14-membered heterocycle.

7. The method of claim 1, wherein R¹¹ is selected from

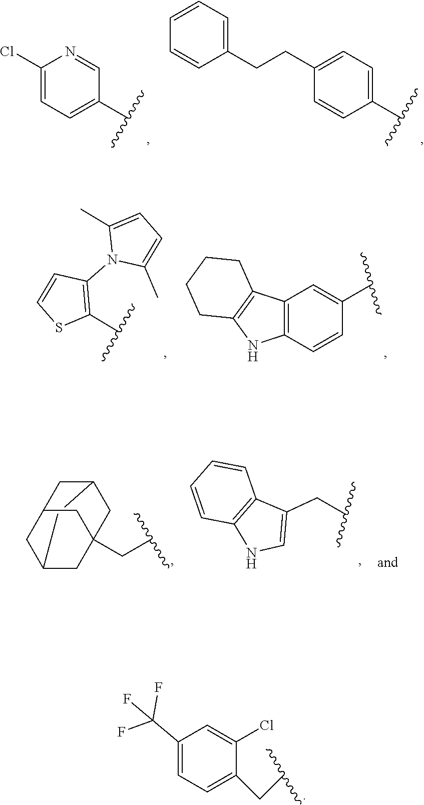

8. The method of claim 1, wherein R²¹ is selected from —OH, —CH₃, and —OCH₂C(O)NHR²², wherein R²² is C₂ alkyl.

9. (canceled)

10. (canceled)

11. (canceled)

12. (canceled)

13. The method of claim 1, wherein R²¹ is —S(O)₂NHR²², wherein R²² is 5- to 14-membered heterocycle.

14. The method of claim 13, wherein R²¹ is

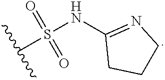 [SMILES: *S(=O)(=O)NC1=NCCC1]

15. The method of claim 1, wherein R²¹ is —NHC(O)R²², wherein R²² is C₃-C₁₂ carbocycle, and wherein R²¹ is

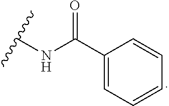 [SMILES: *NC(=O)c1ccccc1]

16. (canceled)

17. (canceled)

18. (canceled)

19. (canceled)

20. The method of claim 1, wherein p is selected form 1, 2, and 3.

21. (canceled)

22. The method of claim 1, wherein the compound having a structural Formula (III) is 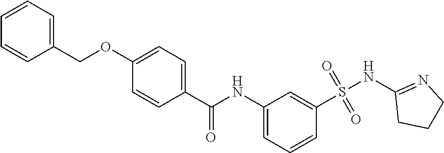 [SMILES: O=C(Nc1cccc(S(=O)(=O)NC2=NCCC2)c1)c1ccc(OCc2ccccc2)cc1]
or a salt thereof.

23. The method of claim 1, wherein the compound having a structural Formula (III) is 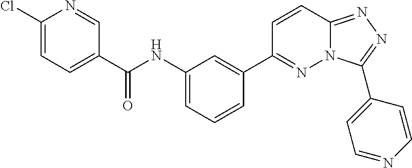 [SMILES: O=C(Nc1cccc(-c2ccc3nnc(-c4ccncc4)n3n2)c1)c1ccc(Cl)nc1]
or a salt thereof.

24. The method of claim 1, wherein the compound having a structural Formula (III) is 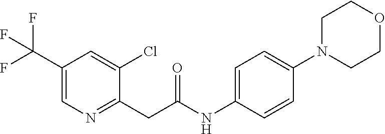 [SMILES: O=C(Cc1ncc(C(F)(F)F)cc1Cl)Nc1ccc(N2CCOCC2)cc1]
or a salt thereof.

25. The method of claim 1, wherein the compound having a structural Formula (III) is 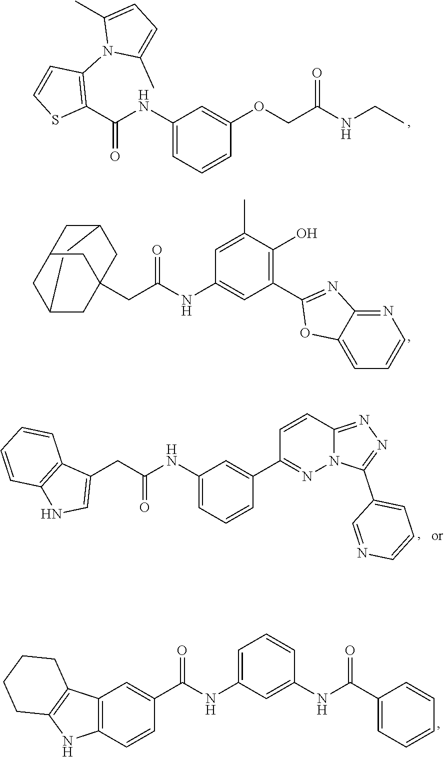 [SMILES: CCNC(=O)COc1cccc(NC(=O)c2sccc2-n2c(C)ccc2C)c1 ; Cc1cc(NC(=O)CC23CC4CC(CC(C4)C2)C3)cc(-c2nc3ncccc3o2)c1O ; O=C(Cc1c[nH]c2ccccc12)Nc1cccc(-c2ccc3nnc(-c4cccnc4)n3n2)c1 ; O=C(Nc1cccc(NC(=O)c2ccc3[nH]c4c(c3c2)CCCC4)c1)c1ccccc1]
or a salt thereof.

26. The method of claim 1, wherein the disease, disorder, or condition comprises a disease, disorder, or condition of skin comprising eczema.

27. (canceled)

28. The method of claim 1, wherein the disease, disorder, or condition comprises a peripheral immune/inflammatory condition comprising atopic dermatitis, contact dermatitis, dyshidrotic eczema, neurodermatitis, nummular eczema, seborrheic dermatitis, stasis dermatitis, vitiligo, or alopecia.

29. (canceled)

30. The method of claim 26, wherein the skin comprises scalp skin, face skin, neck skin, hand skin, penile skin, skin around joints, wrist skin, ankle skin, eyelid skin, skin on and behind the knee, skin on and behind the elbow, feet skin, leg skin, chest skin, abdomen skin, back skin, pelvis skin, flank skin, waist skin, or general areas of skin that rub against each other.

31. The method of claim 1, wherein the administration (i) reduces dryness, itching, redness, roughness, or flakiness of skin, (ii) decreases the expression of interleukin 4 (IL-4) or interleukin 13 (IL-13), or (iii) blocks or reduces engagement between a receptor complex that is composed of interleukin 4 receptor, alpha (IL-4Rα), and an interacting cytokine (i.e., IL-4, IL-13, etc.), or any combination of (i)-(iii).

32. (canceled)

33. (canceled)

34. (canceled)

35. (canceled)

36. (canceled)

37. The method of claim 1, wherein the composition further comprises an additive selected from the group consisting of pharmaceutically acceptable carriers, excipients, adjuvants, diluents, and combinations thereof.

38. The method of claim 1, wherein the composition is formulated as a form of an aerosol, a liquid, a gel, a semisolid, a solid, or a powder, and wherein the composition is formulated as a toner, a cream, an emulsion, a lotion, an ointment, a paste, a gel, a suspension, a serum, an oil, a spray, a transdermal patch, a topical patch, a moisturizer, a face mask, a shampoo, a foam, a cleanser, a mousse, or an aerosol.

39. (canceled)

40. The method of claim 1, wherein the composition is formulated for administration by a topical application, a direct injection, an inhalational route, a transdermal route, an intranasal route, an oral route, a sublingual route, a buccal route, an intravenous injection, an intramuscular injection, a subcutaneous injection, a rectal route, or vaginal route.

41.-208. (canceled)
